(19) United States
(12) Patent Application Publication
(10) Pub. No.: US 2003/0084465 A1
(43) Pub. Date: May 1, 2003

(54) Animal model of allergies

(76) Inventors: Hugh A. Sampson, Larchmont, NY (US); Gary A. Bannon, Little Rock, AR (US); A. Wesley Burks, Little Rock, AR (US)

Correspondence Address: Choate, Hall & Stewart, Exchange Place, 53 State Street, Boston, MA, 02109, US

(21) Appl. No.: 09/989,658

(22) Filed: May 24, 2002

Related U.S. Application Data
(63) Continuation-in-part of application No. 09/518,346, filed on Mar. 3, 2000.
(60) Provisional application No. 60/122,960, filed on Mar. 3, 1999.

(51) Int. Cl. A01K 67/027

(52) U.S. Cl. 800/9; 800/14

(57) ABSTRACT

The present invention provides an animal model for studying allergic reactions to allergens. The animal is sensitized to a selected antigen by administering the antigen itself or a nucleic acid encoding the antigen. Preferred antigens are anaphylactic antigens. The sensitized animal can then be used to screen for compounds which may help to prevent, ameliorate, or cure allergic conditions in humans. A method of sensitizing an animal as well as a method and system for screening chemical compounds is also disclosed.

### RELATED APPLICATIONS

The present application is a continuation-in-part of co-pending application U.S. Ser. No. 09/518,346, filed Mar. 3, 2000, which claims priority to application U.S. Ser. No. 09/455,294, filed Dec. 6, 1999, and also to provisional application U.S. Ser. No. 60/122,960, filed Mar. 3, 1999. Each of these patent applications is incorporated herein by reference in its entirety.

### GOVERNMENT SUPPORT

The invention described herein was supported in part by grants from the National Institutes of Health (Al 43668, AI 24439, AI 33596) and the National Institutes of Environmental Health Sciences (ES03819). The U.S. government retains certain rights in this invention.

### BACKGROUND OF THE INVENTION

Allergic and asthmatic reactions pose serious public health problems worldwide. Pollen allergy alone (allergic rhinitis or hay fever) affects about 10-15% of the population, and generates huge economic costs. For example, reports estimate that pollen allergy generated $1.8 billion of direct and indirect expenses in the United States in 1990 (Fact Sheet, National Institute of Allergy and Infectious Diseases, www.niaid.nih.gov/factsheets/allergystat.html; McMenamin, Annals of Allergy 73:35, 1994). More serious than the economic costs associated with pollen and other inhaled allergens (e.g., molds, dust mites, animal danders) is the risk of anaphylactic reaction observed with allergens such as food allergens, insect venoms, drugs, and latex.

Allergic reactions result when an individual's immune system overreacts, or reacts inappropriately, to an encountered antigen. No allergic reaction occurs the first time an individual is exposed to a particular antigen. However, the initial immune response to an antigen primes the system for subsequent allergic reactions. In particular, the antigen is taken up by antigen presenting cells (e.g., macrophages or dendritic cells) that degrade the antigen and then display antigen fragments to T cells. The activated T cells respond by secreting a collection of cytokines that have effects on other cells of the immune system. The profile of cytokines secreted by responding T cells determines whether subsequent exposures to the particular antigen will induce allergic reactions. When T cells respond by secreting interleukin-4 (IL-4), the effect is to stimulate the maturation of B cells that produce IgE antibodies specific for the antigen. These antigen-specific IgE antibodies then attach to specific receptors on the surface of mast cells and basophils, where they act as a trigger to initiate a rapid reaction to the next exposure to the antigen.

When the individual next encounters the antigen, it is quickly bound by these surface-associated IgE molecules. Each antigen typically has more than one IgE binding site, so that the surface-bound IgE molecules quickly become crosslinked to one another through their simultaneous (direct or indirect) associations with antigen. Such cross-linking induces mast cell degranulation, resulting in the release of histamines and other substances that induce the symptoms associated with allergic reaction. Individuals with high levels of IgE antibodies are known to be particularly prone to allergies.

Current treatments for allergies involve attempts to “vaccinate” a sensitive individual against a particular allergen by periodically injecting or treating the individual with a crude suspension of the raw allergen. The goal is to modulate the allergic response mounted in the individual through controlled administration of known amounts of antigen. If the therapy is successfiul, the individual's allergic response is diminished, or can even disappear. However, the therapy can require several rounds of vaccination, over an extended time period (3-5 years), and very often does not produce the desired results. Moreover, certain individuals suffer anaphylactic reactions to the vaccines, despite their intentional, controlled administration. There is a need for the development of improved treatments for allergies, particularly anaphylactic allergies. including a need for the development of useful model systems in which aspects of allergy and its treatment can be analyzed.

Food Allergies

Food allergies pose particular problems. Not only is the role of anaphylaxis severe with many food allergens, but efforts to develop models for allergic, and particularly anaphylactic, reactions to orally-delivered allergens have generally not been successful. One significant obstacle to the development of food allergy models is the strong innate tendency of animals to develop immunological tolerance to ingested antigens. Various studies in mice have shown that oral tolerance can be influenced by strain (Ito et al. “Murine model of IgE production with a predominant Th2-response by feeding protein antigen without adjuvants” Eur. J. Immunol. 27:3427-3437, 1997; Kiyono et al. “Lack of oral tolerance in C3H/HeJ mice” J. Exp. Med. 155:605-610, 1982; each of which is incorporated herein by reference), age at first feeding (Hanson “Ontogeny of orally induced tolerance to soluble proteins in mice. I. Priming and tolerance in newborns” J. Immunol. 127:1518-1524, 1981; Strobel et al. “Immune responses to fed protein antigens in mice. 3. Systemic tolerance or priming is related to age at which antigen is first encountered” Pediatr. Res. 18:588-594, 1984; Strobel “Neonatal oral tolerance” Ann. N.Y. Acad. Sci. 778:88-102, 1996; each of which is incorporated herein by reference), and the dose and nature of antigen (Mowat “The regulation of immune responses to dietary protein antigens” Immunology Today 8:93-98, 1987; Lamont et al. “Priming of systemic and local delayed-type hypersensitivity responses by feeding low doses of ovalburnin to mice” Immunology 66:595-599; 1989; each of which is incorporated herein by reference).

Peanuts are but one example of an anaphylactic food antigen. Peanuts are highly allergenic and may cause severe allergic reactions in sensitized children and adults (Sampson et al. “Food hypersensitivity and atopic dermatitis: evaluation of 113 patients” J. Pediatr. 107:669-675, 1985; Atkins et al. “Evaluation of immediate adverse reactions to foods in adult patients. I. Correlation of demographic, laboratory, and prick skin test data with response to controlled oral food challenge” J. Allergy Clin. Immunol. 75:348-355, 1985; each of which is incorporated herein by reference). The clinical features of peanut allergy are frequently expressed as acute, IgE-mediated reactions following the ingestion of peanuts (Yunginger et al. “Fatal food-induced anaphylaxis” JAMA 260:1450-1452, 1988; Sampson et al. “Fatal and near-fatal anaphylactic reactions to food in children and adolescents [see comments]” N. Engl. J. Med. 327:380-384, 1992; Kemp et al. “Skin test, RAST and clinical reactions to peanut allergens in children” Clin. Allergy 15:73-78, 1985; Bock et al. “Studies of hypersensitivity reactions to foods in infants and children” J. Allergy Clin. Immunol. 62:327-334, 1978; Sampson et al. “Relationship between food-specific IgE concentrations and the risk of positive food challenges in children and adolescents” J. Allergy Clin. Immunol. 100:444-451, 1997; Sampson “Food allergy and the role of immunotherapy [editorial; comment]” J. Allergy Clin. Immunol. 90:151-152, 1992; each of which is incorporated herein by reference). Peanuts and tree nuts together account for the majority of fatal and near fatal food-induced anaphylactic reactions in the United States (Yunginger et al. “Fatal food-induced anaphylaxis” JAMA 260:1450-1452, 1988; incorporated herein by reference). The prevalence of peanut allergies has increased in recent decades (Sampson “Food allergy and the role of immunotherapy [editorial; comment]” J. Allergy Clin. Immunol. 90:151-152, 1992; incorporated herein by reference), and now peanut allergies affect about 1.5 million Americans. Unlike other childhood food allergies such as cow's milk and egg allergies, peanut allergies are rarely outgrown (Fries “Peanuts: allergic and other untoward reactions” Ann. Allergy 48:220-226, 1982; van Asperen et al. “Immediate food hypersensitivity reactions on the first known exposure to the food” Arch. Dis. Child 58:253-256, 1983; Bock et al. “The natural history of peanut allergy” J. Allergy Clin. Immunol. 83:900-904. 1989; each of which is incorporated herein by reference). Given the severity, prevalence, and frequently lifelong persistence of peanut allergies, and the lack of preventive or curative therapy for peanut allergies, there is a particular need to develop new tools for the study of peanut and other food allergies, and to identify new treatments.

Animal models of food allergies, which mimic the physiological and immunological characteristics of food allergies in man, would be valuable tools in the development of novel immunotherapeutic strategies; however, to date there have been no completely suitable animal models of food allergies to test the efficacy and safety of immunologic therapies. Ermel et al. have reported a dog model for studying food allergies which lead to inflammatory gastrointestinal tract diseases (Ermel et al. “The Atopic Dog: A Model for Food Allergy” Lab. Animal Science 47(l):40-49, 1997). This model is of limited use, however, since the dogs have altered immune systems (i.e., the dogs are from an inbred colony of high IgE-producing dogs) and do not exhibit symptoms of a systemic reaction when challenged with the offending antigen (ie., anaphylaxis, difficulty breathing). An animal model of allergies which will have an anaphylactic reaction upon exposure to the offending antigen would be very useful in designing and testing new strategies for desensitizing allergic patients and in studying the allergic response.

### SUMMARY OF THE INVENTION

The present invention provides an animal model for studying the immune response to allergens. In particular, the invention provides an animal that is sensitized to an antigen so that, when the animal is exposed to the antigen, the exposure elicits an allergic reaction similar to the one seen in humans allergic to the antigen. Preferably, the animal responds to orally-delivered antigen. The animal may be sensitized to antigens such as food antigens (e.g., peanuts, shellfish, fruit, berries), environmental antigens (e.g., dust mites, tree pollens, grass pollens, fungi, animal dander), etc. In certain preferred embodiments of the invention, the animal is sensitized to a food antigen. In some preferred embodiments, when the animal is exposed to the antigen, the animal undergoes an anaphylactic response. In a particularly preferred embodiment, while sensitive to some antigens, the animal is not sensitive to non-allergenic antigens and cannot easily be made sensitive to such non-allergenic antigens. Non-allergenic antigens are typically antigens that humans do not mount allergic responses (i.e., Th2 responses) to. In a particularly preferred embodiment, the animal can not be made sensitive to foods that humans are rarely allergic to (e.g., corn).

Another aspect of the invention comprises a method of sensitizing an animal to an antigen in order to cause an allergic response. In general, the antigen of interest, optionally along with an appropriate adjuvant (e.g., cholera toxin) is administered intragastrically to the animal at least once and preferably two or three times over a week to month to provide the desired response in the animal. In certain preferred embodiments, the animal'gastrointestinal tract or a portion thereof is inflamed. For example, the animal may be infected with an organism that targets the digestive system (e.g., bacteria, viruses, parasites, fungi) and leads to an immune response and inflammation of the infected tissue. In a particularly preferred embodiment, the animal is infected with mouse mammary tumor virus (MMTV) or a variant thereof. Alternatively or additionally, one or more non-viral gastrointestinal irritating agents (e.g., ethanol) is employed.

Preferably, the animal becomes sensitized so that subsequent exposure to the antigen, preferably by the route through which an individual would naturally encounter the antigen, generates a response that is similar to the individual's (preferably a human's) allergic response to the same antigen. The inventive method allows the production of animals sensitized to any desired antigen, and is particularly useful for the generation of animals sensitized to antigens that cause allergies in humans, particularly, food antigens. Furthermore, the method can be applied to any animal, preferably a mammal. In particular embodiments, the method is applied to rodents, such as mice or rats, or to primates, such as apes and monkeys. This method could also be used to sensitize a human being to an antigen, thereby, inducing allergies. Sensitization of the animal may involve administration of antigen in a crude or purified form. Where the antigen is a protein antigen, it may be administered as an intact protein, a peptide, or a polynucleotide encoding the antigenic protein or peptide.

The present invention also provides a system and method of identifying agents that affect the development and/or maintenance of an allergic response. The system comprises an animal sensitized or to be sensitized, test compounds, antigen, and, optionally, an adjuvant. Test compounds are administered to an inventive sensitized animal before, during, and/or after the sensitization process in order to assess their effect on the allergic response. Given the tremendous need for agents which prevent or lessen the symptoms of allergies, this method of using an animal model is of great utility.

In another aspect, the invention provides an animal model for studying skin disorders. In particular, in some embodiments of the invention, a sensitized animal is provided that, when contacted with low doses (i.e., doses below those needed to elicit anaphylaxis or other systemic reactions) of the sensitizing antigen, develops a skin disorder. The skin disorder may be characterized by hair loss, scratching, redness, warmth, exfoliation, etc. In a particularly preferred embodiment, the animal is sensitized to a milk antigen. The present invention also discloses methods of producing such an animal with a skin disorder and methods of identifying compounds used to treat the skin disorder.

The present invention also provides a method of detecting the presence of an antigen in a product. A sensitized animal, as described in the present invention, is contacted with a product to be tested. The immune response of the animal is assessed to evaluate whether the product contains the antigen to which the animal has been sensitized. This method may be particularly important for the food, cosmetic, and drug industries because it allows one to screen products for minute amounts of antigen before they are released to the public.

Definitions

“Allergen”: An “allergen” is an antigen that (i) elicits an IgE response in an individual; and/or (ii) elicits an asthmatic reaction (e.g., chronic airway inflammation characterized by eosinophilia, airway hyperresponsiveness, and excess mucus production), whether or not such a reaction includes a detectable IgE response. Preferred allergens for the purpose of the present invention are protein allergens, although the invention is not limited to such. An exemplary list of protein allergens is presented as an Appendix. This list was adapted on Jul. 22, 1999, from ftp://biobase.dk/pub/who-iuis/allergen.list, which provides lists of known allergens.

“Allergic reaction”: An allergic reaction is a clinical response by an individual to an Hi antigen. Symptoms of allergic reactions can affect the cutaneous (e.g., urticaria, angioedema, pruritus), respiratory (e.g., wheezing, coughing, laryngeal edema, rhinorrhea, watery/itching eyes), gastrointestinal (e.g., vomiting, abdominal pain, diarrhea), and/or cardiovascular (if a systemic reaction occurs) systems. For the purposes of the present invention, an asthmatic reaction is considered to be a form of allergic reaction.

“Anaphylactic antigen”: An “anaphylactic antigen” according to the present invention is an antigen that is recognized to present a risk of anaphylactic reaction in allergic individuals when encountered in its natural state, under natural conditions. For example, for the purposes of the present invention, pollens and animal danders or excretions (e.g., saliva, urine) are not considered to be anaphylactic antigens. On the other hand, food antigens, insect antigens, drugs, and rubber (e.g., latex) antigens are generally considered to be anaphylactic antigens. Food antigens are particularly preferred anaphylactic antigens for use in the practice of the present invention. Particularly interesting anaphylactic antigens are those (e.g., nuts including peanuts, seeds, insect venom, and fish) to which reactions are commonly so severe as to create a risk of death.

“Anaphylaxis” or “anaphylactic reaction”: “Anaphylaxis” or “anaphylactic reaction”, as used herein, refers to an immune response characterized by mast cell degranulation secondary to antigen-induced cross-linking of the high-affinity IgE receptor on mast cells and basophils with subsequent mediator release and the production of pathological responses in target organs, e.g., airway, skin, digestive tract, and cardiovascular system. As is known in the art, the severity of an anaphylactic reaction may be monitored, for example, by assaying cutaneous reactions, puffiness around the eyes and mouth, and/or diarrhea, followed by respiratory reactions such as wheezing and labored respiration. The most severe anaphylactic reactions can result in loss of consciousness and/or death.

“Animal”: The term animal, as used herein, refers to non-human animals, including, for example, mammals, birds, reptiles, amphibians, and fish. Preferably, the non-human animal is a mammal (e.g., a rodent, a mouse, a rat, a rabbit, a monkey, a dog, a cat, or a pig). An animal may be a transgenic animal.

“Antigen”: An “antigen” is (i) any compound or composition that elicits an immune response; and/or (ii) any compound that binds to a T cell receptor (e.g., when presented by an MHC molecule) or to an antibody produced by a B-cell. Those of ordinary skill in the art will appreciate that an antigen may be a collection of different chemical compounds (e.g., a crude extract or preparation) or a single compound (e.g., a protein). Preferred antigens are protein antigens, but antigens need not be proteins for the practice of the present invention.

“Associated with”: When two entities are “associated with” one another as described herein, they are linked by a direct or indirect covalent or non-covalent interaction. Preferably, the association is covalent. Desirable non-covalent interactions include hydrogen bonding, van der Waals interactions, hydrophobic interactions, magnetic interactions, electrostatic interactions, etc.

“Effective amount”: The “effective amount” of an agent (e.g., a pharmaceutical composition, sensitizing composition) refers to the amount necessary to elicit the desired biological response. In the case of sensitizing an animal, the effective amount of a sensitizing composition will cause the desired change in the animal's immune response so that when the animal is contacted with antigen later, the animal will have an immune response. In the case of producing an anaphylactic reaction in an animal, the effective amount of antigen is the amount necessary to produce an anaphylactic reaction (i.e., drop in blood pressure, vasodilatation, difficulty breathing, etc.). In the case of producing a skin disorder in the sensitized animal, the effective amount is the amount necessary to produce the skin disorder without producing other systemic effect. The effective amount to elicit the skin disorder is typically less than the effective amount to sensitize the animal and less than the amount to induce anaphylaxis. The effective amount of antigen to desensitize the sensitized animal is the amount to cause tolerance to the antigen. This amount is typically less than the amount to cause anaphylaxis or sensitization.

“Fragment”: An antigen “fragment” according to the present invention is any part or portion of the antigen that is smaller than the entire, intact antigen. In preferred embodiments of the invention, the antigen is a protein and the fragment is a peptide.

“Gastrointestinal irritating agent”: A gastrointestinal irritating agent is any chemical compound which leads to inflammation or irritation of the gastrointestinal tract. In a preferred embodiment, the chemical compound is a small organic molecule (e.g., ethanol). The inflammation may be limited to a particular portion of the GI tract or to a particular layer (e.g., mucosa, submucosa) of the wall of the GI tract. Preferably, the inflammation leads to the recruitment of inflammatory cells into the wall of the gastrointestinal tract. These cells presumably contribute to the presentation of the administered antigen and help lead to the development of an allergic response to the administered antigen. In another preferred embodiment, the gastrointestinal irritating agent leads to increased permeability of the wall of the gastrointestinal tract and of the blood vessels within the GI tract.

“IgE binding site”: An IgE binding site is a region of an antigen that is recognized by an anti-antigen IgE immunoglobulin. Such a region is necessary and/or sufficient to result in (i) binding of the antigen to IgE; (ii) cross-linking of anti-antigen IgE; (iii) degranulation of mast cells containing surface-bound anti-antigen IgE; and/or (iv) development of allergic signs and symptoms (e.g., histamine release). In general, IgE binding sites are defined for a particular antigen or antigen fragment by exposing that antigen or fragment to serum from allergic individuals. It will be recognized that different individuals may generate IgE that recognize different epitopes on the same antigen. Thus, it is typically desirable to expose antigen or fragment to a representative pool of serum samples. For example, where it is desired that sites recognized by human IgE be identified in a given antigen or fragment, serum is preferably pooled from at least 5-10, preferably at least 15, individuals with demonstrated allergy to the antigen. Those of ordinary skill in the art will be well aware of useful pooling strategy in other contexts.

“Mast cell”: As will be apparent from context, the term “mast cell” is often used herein to refer to one or more of mast cells, basophils, and other cells with IgE receptors.

“Non-allergenic antigen”: A “non-allergenic antigen” is an antigen to which allergic reactions are commonly not observed in humans. Typically, allergic reactions to non-allergenic antigens are seen in less than 10% of the population, more preferably less than 5%, and most preferably less than 1%. In certain preferred embodiments, the non-allergenic antigen when administered to the animal as a sensitizing composition does not lead to sensitization of the animal to the antigen. In a particularly preferred embodiment, exposure to a non-allergenic antigen leads to a Th1 response rather than a Th2 response. In another preferred embodiment, an animal has less than 0. 1% of its IgE directed to the non-allergenic antigen, more preferably less than 0.001%, and most preferably less than 0.0001%. In another particularly preferred embodiment, the animal does not respond to the non-allergenic antigen upon exposure with allergic symptoms such as itching, diarrhea, anaphylaxis, water eyes, rhinorrhea, etc.

“Peptide”: According to the present invention, a “peptide” comprises a string of at least three amino acids linked together by peptide bonds. Peptide may refer to an individual peptide or a collection of peptides. Inventive peptides preferably contain only natural amino acids, although non-natural amino acids (i.e., compounds that do not occur in nature but that can be incorporated into a polypeptide chain; see, for example, http:/www.cco.caltech.edu/dadgrp/Unnatstruct.gif, which displays structures of non-natural amino acids that have been successfully incorporated into functional ion channels) and/or amino acid analogs as are known in the art may alternatively be employed. Also, one or more of the amino acids in an inventive peptide may be modified, for example, by the addition of a chemical entity such as a carbohydrate group, a phosphate group, a farnesyl group, an isofarnesyl group, a fatty acid group, a linker for conjugation, functionalization, or other modification, etc.

“Polynucleotide” or “oligonucleotide”: Polynucleotide or oligonucleotide refers to a polymer of nucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyladenosine, 5-methylcytidine, C5-bromouridine, C5-fluorouridine, C5-iodouridine, C5-propynyl-uridine, C5-propynyl-cytidine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-hydroxylribose, 2′-fluororibose, ribose, 2′-deoxyribose, and hexose), or modified phosphate groups (e.g, phosphorothioates and 5′-N-phosphoramidite linkages).

“Sensitized animal”: A “sensitized animal” is an animal having adapted an immunological state so that, when it encounters an antigen, it has a response similar to that observed in allergic humans. In one preferred embodiment, the initial reaction to the antigen consists primarily of cutaneous reactions with puffiness around the eyes and mouth, and/or diarrhea followed by respiratory reaction such as wheezing and labored respiration. In another preferred embodiment, the percentage of degranulated mast cells is significantly higher in the sensitized animal versus the unsensitized animal. In yet another preferred embodiment, plasma histamine levels were significantly increased in the sensitized animal when it was challenged with antigen. In another preferred embodiment, there is an increased level of antigen specific IgG1 antibodies in the animal after sensitization. In a particularly preferred embodiment of the invention, the response is mediate by IgE immunoglobulin. In another preferred embodiment, the response is an anaphylactic reaction. In preferred embodiments of the invention, the animal's response is similar to that observed in allergic humans who encounter the antigen by the same route (e.g., oral) as that through which the antigen is administered to the animal.

“Sensitized mast cell”: A “sensitized mast cell” is a mast cell that has surface-bound antigen specific IgE molecules. The term is necessarily antigen specific. That is, at any given time, a particular mast cell will be “sensitized” to certain antigens (those that are recognized by the IgE on its surface) but will not be sensitized to other antigens.

“Susceptible individual”: According to the present invention, a person is susceptible to a severe and/or anaphylactic allergic reaction if (i) that person has ever displayed symptoms of allergy after exposure to a given antigen; (ii) members of that person's genetic family have displayed symptoms of allergy against the allergen, particularly if the allergy is known to have a genetic component; and/or (iii) antigen-specific IgE are found in the individual, whether in serum or on mast cells.

“Th1 response” and “Th2 response”: Th1 and Th2 responses are well-established alternative immune system responses that are characterized by the production of different collections of cytokines and/or cofactors. For example, Th1 responses are generally associated with the production of cytokines such as IL-1β, IL-2, IL-12, IL-18, IFNα, IFNγ, TNFβ, etc.; Th2 responses are generally associated with the production of cytokines such as IL-4, IL-5, IL-10, etc. The extent of T cell subset suppression or stimulation may be determined by any available means including, for example, intra-cytoplasmic cytokine determination. In preferred embodiments of the invention, Th2 suppression is assayed, for example, by quantitation of IL-4, IL-5, and/or IL-13 in stimulated T cell culture supernatant or assessment of T cell intra-cytoplasmic (e.g., by protein staining or analysis of mRNA) IL-4, IL-5, and/or IL-13; Th1 stimulation is assayed, for example, by quantitation of IFNα, IFNγ, IL-2, IL-12, and/or IL-18 in activated T cell culture supernatant or assessment of intra-cytoplasmic levels of these cytokines.

### BRIEF DESCRIPTION OF THE DRAWINGS

FIG. 1 shows serum levels of cow's milk—(CM)—specific IgE in a milk-allergic mouse model. Sera from different groups of mice (n =5) as indicated were obtained weekly after CM and cholera toxin (CT) sensitization. CM-specific IgE levels in pooled sera from each group were determined by ELISA. Values are expressed as means ±SEM. *P<0.01 versus #.

FIG. 2 shows systemic anaphylactic symptom scores in milk-allergic mice. Mice (n=5 to 11) were challenged intragastrically with CM. Thirty to 40 minutes later, the symptoms of anaphylaxis were scored on a scale from 0 (no symptoms) to 5 (death), as described in the Methods section. Open circles indicate individual mice. *P<0.001 versus, #; *P<0.05 versus ##.

FIG. 3 shows degranulation of mast cells in milk-allergic mouse ear samples. Panel A shows degranulated mast cells in CM-sensitized (1 mg/g plus CT) mice after challenge (arrows). Panel B shows nondegranulated mast cells in sham-sensitized mice after challenge (arrows). Bar=100 u. Panel C shows percentage of degranulated mast cells in ear samples of CM-sensitized mice, CT sham-sensitized mice, and naive mice. Two hundred to 400 mast cells were analyzed as described in the Methods section. Values are expressed as means ±SEM of 4 mice per group. *P<0.001 versus#.

FIG. 4 shows peanut (PN) antigen-induced systemic anaphylaxis. Mice (n=8-16) were sensitized ig with ground whole PN, 5 mg or 25 mg respectively plus CT. Mice were challenged with crude PN extract 10 mg/mouse in 2 doses at 30-40 min. intervals at week 3(A). Thirty to forty min. following challenge, the symptoms of anaphylaxis were scored utilizing a scoring system as described in Materials and Methods. Mice surviving the first challenge at week 3 were rechallenged at week 5(B), and the symptoms scored as above. Symbol (open circle) indicates individual mice. ^{#} p<0.05 vs. high dose group. Data are combined results of 3-4 individual experiments.

FIG. 5, panel A shows degranulation of mast cells. Ear samples were collected immediately after anaphylaxis-related death or 40 min. after challenge of surviving mice and fixed. Five μm toluidine blue or Giemsa stained paraffin sections were examined by light microscopy at 400×. Four hundred mast cells were classified for each ear sample. Values are expressed as means ±SEM of 3-4 mice per group. ^{#} p<0.001 vs. controls. Panel B shows plasma histamine levels. Thirty min. following PN-challenge, blood from each group of mice (n=4) was collected, and histamine levels were determined using a commercial enzyme immunoassay kit. ^{#} p<0.05 vs. controls.

FIG. 6 shows the concentration of PN-specific IgE. Sera from different groups of mice (n=8-16) as indicated were obtained weekly following initial PN-sensitization. Ara h 2-specific IgE levels were determined by ELISA. Data are given as mean ±SEM of 3-4 experiments.

FIG. 7 shows the splenocyte proliferative response to PN, Ara h 1, and Ara h 2 stimulation. Spleen cells from PN allergic mice (n=2) and naive mice (n=2) were stimulated with 10 and 50 μg/ml of crude PN extract, Ara h 1, or Ara h 2. Cells cultured in medium alone or with Con A served as controls. Four days later, the cultures received an 18-hr pulse of 1 μCi per well of ³H-thymidine. The cells were harvested and the incorporated radioactivity was counted. The results are expressed as counts per minute (cpm).

FIG. 8 shows the concentration of PN, Ara h 1, and Ara h 2-specific IgE. Pooled sera from PN-allergic mice or naive mice (n=6) were prepared. The levels of PN, Ara h 1, and Ara h 2-specific IgE were determined by ELISA.

FIG. 9 shows a comparison of mouse and patient IgE antibody binding to Ara h 2 isoforms. Crude PN protein extract (200 μg) resolved by two-dimensional SDS-PAGE on nitrocellufose membranes was probed with pooled sera from PN-allergic patients (A) or PN-allergic mice (B) as described in Example 2 under Materials and Methods.

FIG. 10 shows levels of Ara h 2-specific Abs in C3H mice. A, Levels of Ara h 2-specific IgG2a. B. Levels of Ara h 2-specific IgG1. Sera from different groups of mice (n=4-7) as indicated were obtained 3 wk after pDNA immunization. The levels of Ara h 2-specific IgG2a and IgG1 were determined by ELISA, and calculated by comparison with a references curve generated using mouse monoclonal anti-DNP Abs. *p<0.05 versus pcDNA, sin; **,p <0.001 versus pcDNA, mul.

FIG. 11 shows peanut-induced anaphylaxis in C3H mice. Three weeks following the initial pDNA immunization mice (n =4-7) in each group received an i.p. injection of PN, or Ara h 2 or CA. The severity of anaphylaxis was scored 20-40 min after i.p. Ag administration, as described in Materials and Methods. *, **, p<0.001 versus pcDNA; **p <0.01 versus pAra h 2 sin.

FIG. 12 shows plasma histamine level following PN injection of C3H mice. Five to eight minutes after PN injection, plasma from different groups of mice, as indicated (n =4-5), was obtained. The level of histamine was measured by ELISA, and calculated by comparison with a standard curve *, p<0.01 versus pcDNA.sin: **,p<0.001 versus pcDNA, mul.; **, p <001 versus pAra h 2, sin.

FIG. 13 shows the percentage of degranulated mast cells in ear samples of pAra h 2 and mock DNA-immunized mice (200-400 mast cells were analyzed). *, p<0.01 versus pcDNA, sin; **, p<0.001 versus pcDNA.mul.; **, p<0.01 versus pAra h 2, sin.

FIG. 14 shows ovomucoid-specific Abs induced by pOMC in C3H mice. Sera from different groups of mice (n=5), as indicated, were obtained at weekly intervals from 1-3 wk after the initial pDNA immunization. The levels of ovomucoid-specific IgG2a and IgG1 were WQ determined by ELISA and calculated by comparison with a reference curve generated using mouse mAb, anti-DNP Abs.

FIG. 15 shows kinetics of isotype profile of Ara h 2-specific Abs induced by pAra h 2 immunization of AKR, BALB/c, and C3H mice A, Levels of Ara h 2-specific IgG2a B. Levels of Ara h 2-specific IgG1. Sera from different groups of mice (n=5) were obtained at weekly intervals following multiple pDNA immunization. The levels of Ara h 2-specific IgG2a and IgG1 were determined by ELISA and calculated by comparison with a reference curve generated by using mouse mAb anti-DNPAbs.

FIG. 16 shows the ability of each peptide of Ara h 2 to stimulate T cells. Each peptide was tested, using standard techniques, on 19 different T cell preparations. Positive scores, defined as a T cell stimulation index of >2, are indicated by shading.

FIG. 17 shows the modified amino acid sequences of Ara h 1, Ara h 2, and Ara h 3. Altered positions are underlined.

FIG. 18 shows a decrease in Ara h 2-specific IgE in blood of mice desensitized with modified Ara h 2 protein.

### DETAILED DESCRIPTION OF CERTAIN PREFERRED EMBODIMENTS OF THE INVENTION

The present invention provides a sensitized animal model of allergic reactions and methods of making and using such a model. The antigen of interest is administered to the animal, optionally in combination with one or more adjuvants or other factors, until the animal becomes appropriately sensitized. As discussed in more detail below, the antigen may be administered by any of a variety of routes and according to any of a variety of protocols. The sensitized animal can then be used as an experimental model for an allergic response to the administered antigen, and also as a system for the identification of chemical compounds that affect the development and/or maintenance of allergic reactions. Of particular interest are those compounds that can be used to vaccinate against the development of allergy (i.e., can block or retard development of the sensitized state when administered prior to or during sensitization), and/or to reverse allergic sensitization after it has occurred.

Animal

The animal sensitized to a particular antigen and used in this invention can be any non-human animal from the animal kingdom. Preferably, the animal is a mammal, and more preferably, the animal is a rodent. Examples of rodents include mouse, rat, rabbit, ferret, hamster, gerbil, guinea pig, etc. In other preferred embodiments of the invention, the animal used is a non-human primate. Non-human primates include apes, monkeys, orangutans, baboons, etc.

The animal used in the present invention also may be a transgenic animal. The term transgenic animal is meant to include an animal whose genome has been altered by the hand of man. For instance, the transgenic animal may have gained new genetic material from the introduction of foreign DNA, i.e., partly or entirely non-naturally occurring in the recipient organism, into the DNA of its cells; or introduction of a lesion, e.g., an in vitro induced mutation, e.g., a deletion or other chromosomal rearrangement into the DNA of its cells; or introduction of homologous DNA into the DNA of its cells in such a way as to alter the genome of the cell into which the DNA is inserted, e.g., it is inserted at a location which differs from that of the natural gene or its insertion results in a knockout. The animal may include a transgene in all of its cells including germ line cells, or in only one or a portion of its cells.

In certain preferred embodiments, the animal has inflammation of its gastrointestinal tract. The inflammation may be caused by any etiology including, but not limited to, exposure to noxious chemicals, viral infection, bacterial infection, fungal infection, protozoan infection, genetic defects, autoimmunity, generalized immuno-overactivity, etc. In a particularly preferred embodiment, the animal is infected with mouse mammary tumor virus (MMTV) or a variant thereof. Without wishing to be bound by a particular theory or explanation, the immune response in the inflamed tissue (e.g., digestive tract, lungs) may set up the proper environment for sensitizing the animal to a particular antigen. Preferably, the antigen is delivered in such a way as to come in contact with the inflamed tissue. For example, an inflamed digestive tract may lead to the sensitizing composition being delivered orally or intragastically. To give but one other example, inflamed lung tissue may be caused by a respiratory virus known to infect the animal and cause inflammation and may allow for delivery of the sensitizing composition inhalationally.

In a particularly preferred embodiment, the animal's immune system responds in a manner very similar to a human's immune system. For example, humans are known to be rarely, if ever, allergic to certain food antigens (e.g., corn); therefore, the animal used in this method would ideally not be able to be sensitized to such food antigens. In a particularly preferred embodiment, the animals sensitized to a particular antigen are not allergic and/or cannot be made allergic to non-allergenic antigens. For example, following the methods outlined below in the Examples, one would not be able to sensitize an animal to a non-allergenic antigen. When compared to controls, there would be little to no increase in antigen-specific IgE levels, little to no increase in basophil histamine release upon exposure to the non-allergenic antigen, little to no clinical response, little to no mast cell degranulation, etc.

Additional characteristics that may influence the selection of a particular animal for sensitization in accordance with the present invention include, for example, similarity of the animal's immune system to the human immune system, ease of care of the animal, past experience and knowledge of the animal as an experimental model, cost of the animal, cost of caring for the animal, similarity of allergic response of animal compared to human allergic response, inability of the animal to be sensitized to antigens that are typically non-allergenic to humans, etc.

Sensitizing Antigen

In general, any antigen may be employed to sensitize an animal in accordance with the present invention, so long as, when it is administered, it results in a sensitized animal. The sensitized animal should have an immunological response when it encounters the antigen, and in preferred embodiments, the response should be similar to that observed in allergic humans, and more preferably, should be an IgE-mediated response. Preferred antigens are protein antigens. The Appendix presents a representative list of certain known protein antigens. As indicated, the amino acid sequence is known for many or all of these proteins, either through knowledge of the sequence of their cognate genes or through direct knowledge of protein sequence, or both.

Of particular interest are anaphylactic antigens. Anaphylactic antigens include food antigens, insect antigens, and rubber antigens (e.g., latex). In particular, nut (e.g., peanut, walnut, almond, pecan, cashew, hazelnut, pistachio, pine nut, brazil nut) antigens, dairy (e.g., egg, milk) antigens, seed (e.g., sesame, poppy, mustard) antigens, fish/shellfish (e.g., shrimp, crab, lobster, clams) antigens, and insect antigens are anaphylactic antigens according to the present invention. Particularly preferred anaphylactic antigens are food antigens; peanut (e.g., Ara h 1-3), milk, egg, and fish/shellfish (e.g., tropomyosin) antigens are especially preferred. In some cases, it will be desirable to work in systems in which a single compound (e.g., a single protein) is responsible for most observed allergies. In other cases, the invention can be applied to more complex allergens.

Environmental antigens may also be used in the present invention. Environmental antigens include animal dander, tree pollen, grass pollen, weed pollen, mites, dust mites, animal antigens, insect antigens, and fungal antigens. Specific examples of these antigens are listed in the Appendix.

Sensitizing antigens for use in the present invention may be produced in any desired form. For example, crude antigen may be used, or antigen may be partially or completely pure. In some cases, it will be desirable to sensitize the animal with an antigen composition that approximates as closely as possible the form of the antigen in nature. Such a formulation is not required, however. In some embodiments of the invention, a protein antigen is provided by a polynucleotide encoding the antigen. DNA or RNA may be used in the invention; however, DNA is generally preferred given its greater stability. The polynucleotide may be provided in the context of a delivery vector such as a plasmid or virus. Preferably, the polynucleotide includes expression sequences (e.g., promoter, enhancer, splicing signals, Shine-Delgarno, etc.) sufficient to direct protein expression in the relevant animal. A wide variety of such sequences is known in the art. Once inside a cell, the polynucleotide is transcribed and translated in order to produce the protein antigen in situ. Production of the protein antigen leads to sensitization of the animal. In a preferred embodiment, the expression system (e.g., promoter, enhancer, splicing signals, etc.) and vector are matched to the species being sensitized. For example, if a mammal such as a mouse was to be the experimental model, the promoter used to drive protein production might be the cytomegalovirus (CNV) promoter.

Multiple sensitizing antigens may be provided together or in series to generate an animal sensitized to more than one antigen. For example, it may be desirable to sensitize animals to multiple different antigens that are found in nature in a single source. For instance, at least seven proteins have been identified in peanuts as potential antigens. Two of these, Ara h 1 and Ara h 2, are recognized by more than 95% of peanut allergic patients (Burks et al. Identification of soy protein allergens in patients with atopic dermatitis and positive soy challenges determination of change in allergenicity after heating or enzyme digestion” Adv. Exp. Med. Biol. 289:295-307, 1991; Burks et al. “Identification and characterization of a second major peanut allergen, Ara h II, with use of the sera of patients with atopic dermatitis and positive peanut challenge” J. Allergy Clin. Immunol. 90:962-969, 1992; Shin et al. “Biochemical and structural analysis of the IgE binding site on ara h 1, an abundant and highly allergic peanut protein” J. Biol. Chem. 273:13753-13759, 1998; each of which is incorporated herein by reference). A third protein, Ara h 3, is recognized by about 45% of peanut allergic patients (Rabjohn et al. “Molecular cloning and epitope analysis of the peanut allergen ara h3 [In process citation]” J. Clin. Invest. 103:535-542, 1999; incorporated herein by reference). In a preferred embodiment, it may be desirable to create an animal sensitive to all these peanut antigens by administering all the antigens together (e.g., in a crude preparation or mixture) or individually. The different antigens may also be administered in different ratios, preferably with more of the less antigenic antigens being administered.

Modified versions of the antigens may also be used in the present invention. Any type of modification can be used. They may be biological or chemical. The antigen may contain unnatural amino acids; may be glycosylated, phosphorylated, hydroxylated, etc.; may be cross-linked; may contain mutations (e.g., substitutions, deletions); etc.

An antigen fragment may also be used in the present invention to create the allergic animal model. An antigen fragment is a portion of the antigen that is smaller than the intact antigen. Compositions including antigen fragments will preferably contain either a sufficiently large number of antigen fragments or at least one antigen fragment that is sufficiently sized that the composition contains one or more immunologically relevant structural elements that are present in the intact antigen. As mentioned above, the antigen is preferably a protein, and the fragment is preferably a peptide. Preferred peptides are at least six amino acids long; particularly preferred peptides are at least about 10, 12, 15, 20, or 30 amino acids long.

Adjuvants

In certain preferred embodiments of the invention, the sensitizing antigens are provided with one or more immune system adjuvants, preferably selected to enhance sensitization. A large number of adjuvant compounds is known; a useful compendium of many such compounds is prepared by the National Institutes of Health and can be found on the world wide web (http:/www.niaid.nih.gov/daids/vaccine/pdf/compendium.pdf, incorporated herein by reference; see also Allison Dev. Biol. Stand. 92:3-11, 1998; Unkeless et al. Annu. Rev. Immunol. 6:251-281, 1998; and Phillips et al. Vaccine 10:151-158,1992, each of which is incorporated herein by reference). Preferred adjuvants are characterized by an ability to stimulate Th2 responses preferentially over Th1 responses and/or to down regulate Th1 responses. In fact, in certain preferred embodiments of the invention, adjuvants that are known to stimulate Th1 responses are avoided. Adjuvants which are known to stimulate Th1 responses include, for example, preparations (including heat-killed samples, extracts, partially purified isolates, or any other preparation of a microorganism or macroorganism component sufficient to display adjuvant activity) of microorganisms such as Listeria monocytogenes or others (e.g., Bacille Calmette-Guerin [BCG], Corynebacterium species, Mycobacterium species, Rhodococcus species, Eubacteria species, Bortadella species, and Nocardia species), and preparations of nucleic acids that include unmethylated CpG motifs (see, for example, U.S. Pat. No. 5,830,877; and published PCT applications WO 96/02555, WO 98/18810, WO 98/16247, and WO 98/40100, each of which is incorporated herein by reference). Other adjuvants reported to induce Th1-type responses and not Th2-type responses include, for example, Aviridine (N,N-dioctadecyl-N′N′-bis (2-hydroxyethyl) propanediamine), and CRL 1005. Preferably, Th2-inducing agents are used including IL-4, aluminum phospate gel (Adju-Phos), Algammulin, aluminum hydroxide gel (Alhydrogel), Bay R1005, cholera toxin, cytokine-containing liposomes, gamma inulin, GM-CSF, Rehydragel HPA, and Rehydragel LV.

In some embodiments of the invention, the adjuvant is associated (covalently or non-covalently, directly or indirectly) with the sensitizing antigen so that adjuvant and antigen can be delivered substantially simultaneously to the individual, optionally in the context of a single composition. In other embodiments, the adjuvant is provided separately. Separate adjuvant may be administered prior to, simultaneouly with, or subsequent to antigen. In certain preferred embodiments of the invention, a separate adjuvant composition is provided that can be utilized with multiple different antigen compositions.

Where adjuvant and antigen are provided together, any association sufficient to achieve the desired immunomodulatory effects may be employed. Those of ordinary skill in the art will appreciate that covalent associations will sometimes be preferred. For example, where adjuvant and antigen are both polypeptides, a fusion polypeptide may be employed. Those of ordinary skill in the art will be aware of other potentially desirable covalent linkages.

Administration

The sensitizing composition (i.e., sensitizing antigen with or without any adjuvant and/or other factor or agent) may be administered to the animal using any available route and any dosing regiment. Known routes of administration included intravenous (IV), intraperitoneal (IP), intragastric (IG), sub-cutaneous (SQ), intramuscular (IM), oral (PO), rectal (PR), intrathecal, vaginal, and intranasal administration. Generally, it is preferred that the animal be sensitized by exposure to the antigen via the same route through which the organism (e.g., human) for which the animal is a model, typically becomes exposed to the antigen in nature. Preferred methods of administering the sensitizing composition include intragastric, intramuscular, oral, and intranasal administration. A most preferable method of administration, particularly for food antigens, is intragastric administration.

The dosing regiment of this invention includes any protocol which sensitizes the animal to the presented antigen. At least one dose of the sensitizing composition is administered to the animal, but the sensitization may include up to fifty administrations of the sensitizing composition. Preferably, less than 10 administrations of the sensitizing composition are required to sensitize the animal. Even more preferably, less than 5, or most preferably less than 3 administrations are required. As appreciated by one skilled in this art, the dosing regiment may depend on a number of factors. These factors include, for example, the antigen being presented, the level of sensitization desired, the adjuvant being used, the method of administration, the animal chosen as the model, the amount of antigen given per administration, etc.

The amount of antigen administered per dose may range from 0.001 mg/g body mass to 10 mg/g body mass. Preferably, the dosage used ranges from 0.01 mg/g body mass to 5 mg/g body mass. More preferably, the dosage used ranges from 0.1 mg/g body mass to 2 mg/g body mass. Those of ordinary skill in the art will be aware of techniques, including those described add herein, for delivering the optimum sensitizing dose and protocol. For example, a known amount of antigen may be administered, followed by measurement of the antigen-specific IgE in sera, assessment of hypersensitivity responses, detection of vascular leakage, determination of plasma histamine levels, testing for passive cutaneous anaphylaxis, quantitation of cytokine proteins, examination of histology of mast cells, and/or determination of serum antigen concentration.

Where an animal is sensitized with a nucleic acid encoding a protein antigen, the preferred amount of DNA per dose ranges 0.01 μg to 1 mg DNA. Preferably, the amount of DNA per dose is 0.1 μg to 100 μg DNA; and more preferably, the amount of DNA per dose is 1 μg to 50 μg DNA. If RNA is to be used as the encoding nucleic acid, more RNA may need to be administered due to the short half-life of RNA in vivo.

The sensitization method may employ a combination of antigen per se and DNA encoding the antigen. They may be administered at the same time or separately. The sensitizing composition may be formulated as a pharmaceutical composition as discussed below.

Identification of Agents that Alter Establishment and/or Maintenance of a Sensitized State

The inventive sensitized animals provide models of allergic reactions that can be used both to analyze the features of allergy and to identify agents that can alter the establishment and/or maintenance of an allergic state.

Agents that can be used in this method include peptides, proteins, polynucleotides, agents secreted by bacteria, modified proteins, modified peptides, collections of peptides, encapuslated antigens, small organic molecules, chemical compounds, lipids, carbohydrates, etc.

In a particularly preferred embodiment of the present invention, the agents are chemical compounds. These chemical compounds may be provided by any known method in the art including traditional synthesis, derviatizing known compounds, using existing libraries of compounds, purchasing known commercially available compounds, combinatorial chemistry, etc. The agents may also be provided in the form of a collection of chemical compounds such as a combinatorial chemistry library. The compounds may be provided in any form (e.g., salt, ester, derivative).

In another particularly preferred embodiment, the agent is a modified protein. The protein has been modified when compared to the wild type sequence so that the IgE binding sites have-been removed or reduced in number. Exemplary agents are further described in patent applications U.S. Ser. No. 09/247,406, filed Feb. 10, 1999; U.S. Ser. No. 09/141,220, filed Aug. 27, 1998; U.S. Ser. No. 09/478,668, filed Jan. 6, 2000; and U.S. Ser. No. 09/240,557, filed Jan. 29, 1999, each of which is incorporated herein by reference.

In another preferred embodiment, the agent comprises a collection of peptides that span the sequence of a known protein antigen. The collection of peptides may be selected so that the IgE binding sites are removed, disrupted, or are of a limited number on any given peptide. Exemplary collections of peptides are described further in U.S. patent application U.S. Ser. No. 09/455,294, filed Dec. 6, 1999, which is incorporated herein by reference (see Examples 2 and 3).

In another preferred embodiment, the agent is a protein antigen which has been encapsulated. The encapsulated protein antigen is hidden from the mast cells while being available to antigen presenting cells for uptake and processing. This approach to desensitization is described in detail in U.S. patent application U.S. Ser. No. 60/169,330, filed Dec. 6, 1999, incorporated herein by reference.

In yet another preferred embodiment, the agent comprises a substance that will occupy all the IgE binding sites of the immune system and thereby prevent binding of the offending IgE. This approach is described in detail in U.S. patent application U.S. Ser. No. 09/090,375, filed Jun. 4, 1998, incorporated herein by reference.

In another preferred embodiment, the agent masks the IgE binding sites of the antigen and does not crosslink IgE. This agent may be given in combination with the offending antigen. These agents are described in U.S. patent application U.S. Ser. No. 09/216,117, filed Dec. 18, 1998, incorporated herein by reference.

In another preferred embodiment, the agent comprises dendritic cells that have been removed from the sensitized individual and treated in such a way as to lead to tolerance for the antigen when they are placed back into the individual. This strategy is described further in U.S. patent application U.S. Ser. No. 09/290,029, filed Apr. 9, 1999, incorporated herein by reference.

In another preferred embodiment, the agent comprises an antigen combined with an adjuvant. The adjuvant is selected to accelerate immunotherapy, e.g., by promoting a Th2 response. U.S. patent application U.S. Ser. No. 09/339,068, filed Jun. 23, 1999, incorporated herein by reference, describes using heat killed Listeria monocytogenes as an adjuvant, and U.S. patent applications U.S. Ser. No. 60/125,071, filed Mar. 17, 1999, and U.S. Ser. No. 60/124,595, filed Mar. 16, 1999, each of which is incorporated herein by reference, describe using nucleotide sequences (e.g., CpG) as an adjuvant.

In another preferred embodiment, the agent comprises a bacterium that produces and/or secretes the offending protein antigen in small quantities. Such bacteria themselves may also act as adjuvants. Such a combination of antigen and adjuvant should lead to desensitization of the treated patient.

Agent(s) to be tested are administered to the inventive sensitized animal before, during, or after the sensitization process. For example, an agent that is found to prevent or reduce an immune response to a particular antigen when the compound is administered before sensitization might be used as a vaccination. Any of the routes described above in administering an antigen may be used to deliver the agent.

Those of ordinary skill in the art will appreciate that any of a variety of assays may be employed to assess the effect of the test agent. These include measurement of serum Ag-specific antibodies, observation of the animal, assessment of hypersensitivity responses, detection of vascular leakage, determination of plasma histamine levels, histologic studies, passive cutaneous anaphylaxis (PCA) test, quantitation of cytokines, and two-dimensional gel electrophoresis and immunoblotting. A person of skill in this art will be familiar with appropriate controls and such techniques for such assays including, for example, testing before and after administration of the agent, and before and after the sensitization process.

Animal Model of Skin Disorders

Certain preferred sensitized animals of the present invention as described herein may be used to study skin disorders. The skin disorder may be characterized by hair loss, redness, warmth, itching, exfoliation, etc. For example, as described below in Example 1, inventive mice sensitized to milk allergen will develop a skin disorder characterized by hair loss when fed chow containing small amounts of the relevant antigen. According to the present invention, an animal model of a skin disorder may be provided by sensitizing an animal to an antigen and then exposing the animal, preferably over a continuous period, to small smounts of the antigen.

Methods of Detecting the Presence of Antigen in a Product

The sensitized animals may be used to detect small quantities of the sensitizing antigen in a product. The sensitized animal is contacted with the product to be tested, and the animal is assessed to determine its immune response to the antigen. This assessment may include laboratory tests or simple observation of the animal. In particular, the assessment may include measurement of serum Ag-specific antibodies, assessment of hypersensitivity responses, detection of vascular leakage, determination of plasma histamine levels, histologic studies, passive cutaneous anaphylaxis (PCA) test, quantitation of cytokines, observation of the animal, assessment of breathing and respiration, and two-dimensional electrophoresis and immunoblotting.

The products that may be tested using this method include any composition of matter. Some examples include food products, cosmetic compostions, personal hygiene products, pharmaceutical compositions, detergents, soaps, plastics, polymers, chemical compounds, perfumes, textiles, gloves, medical devices, etc.

Pharmaceutical Compositions

Pharmaceutical compositions for use in accordance with the present invention may include a pharmaceutically acceptable excipient or carrier. As used herein, the term “pharmaceutically acceptable carrier” means a non-toxic, inert solid, semi-solid or liquid filler, diluent, encapsulating material or formulation auxiliary of any type. Some examples of materials which can serve as pharmaceutically acceptable carriers are sugars such as lactose, glucose, and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. The pharmaceutical compositions of this invention can be administered to humans and/or to other animals, orally, rectally, parenterally, intracisternally, intravaginally, intraperitoneally, topically (as by powders, ointments, or drops), bucally, or as an oral or nasal spray.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsfying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacteria-retaining filter, or by incorporating sterilizing agents in the form of sterile solid compositions which can be dissolved or dispersed in sterile water or other sterile injectable medium prior to use.

In order to prolong the effect of an agent, it is often desirable to slow the absorption of the drug from subcutaneous or intramuscular injection. This may be accomplished by the use of a liquid suspension of crystalline or amorphous material with poor water solubility. The rate of absorption of the agent then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of agent to polymer and the nature of the particular polymer employed, the rate of release of the agent can be controlled. Examples of other biodegradable polymers include poly(orthoesters) and poly(anhydrides) Depot injectable formulations are also prepared by entrapping the drug in liposomes or microemulsions which are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar -agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Solid compositions of a similar type may also be employed as fillers in soft and hard-filled gelatin capsules using such excipients as lactose or milk sugar as well as high molecular weight polethylene glycols and the like.

The compounds can also be in micro-encapsulated form with one or more excipients as noted above. The solid dosage forms of tablets, dragees, capsules, pills and granules can be an prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Dosage forms for topical or transdermal administration of an inventive pharmaceutical composition include ointments, pastes, creams, lotions, gels, powders, solutions, sprays, inhalants, or patches. The active component is admixed under sterile conditions with a pharmaceutically acceptable carrier and any needed preservatives or buffers as may be required. Ophthalmic formulation, ear drops, and eye drops are also contemplated as being within the scope of this invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Transdermnal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

These and other aspects of the present invention will be further appreciated upon consideration of the following Examples, which are intended to illustrate certain particular embodiments of the invention but are not intended to limit its scope, as defined by the claims.

### EXAMPLES

### Example 1

### A Murine Model of Milk Anaphylaxis

Introduction

This Example describes the development of a mouse model system for anaphylactic milk allergy. This system may be employed in accordance with the present invention to identify and characterize compositions capable of desensitizing and/or vaccinating individuals from milk allergy. This system may also be empolyed to study skin disorders when the mice are fed low levels of milk antigen.

Materials and Methods

MICE AND MATERIALS:

Female C3H/HeJ mice, 3 weeks of age (immediately after weaning), were purchased from the Jackson Laboratory (Bar Harbor, Me.) and maintained on regular mouse chow under specific pathogen-free conditions. Guidelines for the care and use of the animals were followed (Institute of Laboratory Animal Resources Conmmission on Life Sciences, National Academy Press, 1996).

Homogenized cow's milk (CM; GAF Seelig Inc) was used. Cholera toxin (CT) was purchased from List Biological Laboratories, Inc (Campbell, Calif.). Concanavalin (Con A) and albumin, human-dinitrophenyl (DNP)-albumin were purchased from Sigma (St Louis, Mo.). Antibodies for ELISAs were purchased from the Binding Site Inc. or PharMingen (San Diego, Calif.). Anti-DNP IgE was purchased from Accurate Scientific Inc.

Sensitization and Challenge by Oral Administration of Antigen:

Mice were sensitized intragastrically with CM plus CT as an adjuvant and boosted 5 times at weekly intervals. Intragastric feeding was performed by means of a stainless steel blunt feeding needle (Fine Science Tool Inc.) To determine the optimum sensitizing dose, mice received 0.01 mg (equivalent to the milk protein contained in homogenized CM) per gram of body weight (very low dose), 0.1 mg/g (low dose), 1.0 mg/g (medium dose), or 2 mg/g (high dose) of CM together if with 0.3 μg/g of CT. The CM/CT mixtures were administered in PBS at a final volume of 0.03 mL/g body weight. Control mice received CT alone or were left untreated. Six weeks after the first feeding, mice were fasted over night and challenged intragastrically with 2 doses of CM (30 mg/mouse) given 30 minutes apart.

Measurement of Cm-Specific Ige in Sera:

Blood was obtained weekly from the tail vein during the sensitization period and 1 day before challenge. Sera were collected and stored at −80° C. Levels of CM-specific IgE were measured by ELISA as described previously (Li et al., J. Immunol. 160:1378-84, 1998). Immulon II 96-well plates (Dynatech Laboratories, Inc. Chantilly, Va.) were coated with 20 μg-mL purified cow milk protein (CMP) (Ross Laboratories, Columbus, Ohio) in coating buffer, pH 9.6 (Sigma). After overnight incubation at 4° C., plates were washed 3 times with PBS/0.05% Tween 20 and blocked with 1% BSA-PBS for 1 hour at 37° C. After washing 3 times, serum samples (1:10 dilutions) were added to the plates and incubated overnight at 4° C. Plates were then washed, and 100 μL of donkey anti-goat IgG antibody conjugated with peroxidase (0.3μg/mL) was added for an additional 1 hour at 37° C. The reactions were developed with TMB (Bio-Rad Laboratories, Hercules, Calif.) for 30 minutes at room temperature (RT), stopped with the addition of 1 N H₂SO₄, and read at 450 nm. The levels of IgE were calculated by comparison with a reference curve generated by using mouse mAbs (anti-DNP IgE), as previously described (Li et al., J. Immunol. 160:1378-84, 1998). All analyses were performed in duplicate.

Assessment of Hypersensitivity Responses:

Symptoms of systemic anaphylaxis appeared within 15 to 30 minutes and reached a peak at 40 to 50 minutes after the first symptoms Ad appeared. Symptoms were evaluated by using a scoring system modified slightly from previous reports and scored as follows: 0=no symptoms; 1=scratching and rubbing around the nose and head; 2=puffiness around the eyes and mouth, pilar erecti, reduced activity, and/or decreased activity with increased respiratory rate; 3=wheezing, labored respiration, and cyanosis around the mouth and the tail; 4=no activity after prodding or tremor and convulsion; and 5=death.

Detection of Vascular Leakage:

Immediately before the second intragastric challenge with CM, 2 to 4 from each group received 100 μL of 0.5% Evan's blue dye by tail vein injection. Footpads and intestines of mice were examined for signs of vascular leakage (visible blue color) 30 to 40 minutes after dye/antigen administration.

Determination of Plasma Histamine Levels:

Thirty minutes after challenge, blood was collected into chilled tubes containing 30 to 40 μL of 7.5% potassium-EDTA. After centrifugation (1500 rpm) for 10 minutes at 4° C., plasma aliquots were collected and frozen at −80° C. Histamine levels were determined by using an enzyme immunoassay kit (ImmunoTECH Inc), as described by the manufacturer.

Passive Cutaneous Anaphylaxis (Pca) Test:

Sera were obtained from 4 to 6 mice sensitized to CM (1 mg/g) plus CT and pooled. PCA tests were performed as previously described (Saloga et al., J. Clin. Invest. 91:133-40, 1993), with slight modification. Briefly, the abdomens of naive mice were shaved 1 day before intradermal injection of 50 μL of heated (56° C. for 3 hours) and unheated sera (1:5 dilution). Control mice received an equal amount of diluted naive serum. Twenty four hours later, mice were injected intravenously with 100 μL of 0.5% Evan's blue dye, immediately followed by an intradermal injection of 50 μL of CMP (4 mg/mL). Thirty minutes after the dye/CMP injection, the mice were killed, the skin of the belly was inverted, and reactions were examined for visible blue color. A reaction was scored as positive if the bluing of the skin at the injection sites was greater than 3 mm in diameter in any direction.

Determination of Serum Antigen Concentration:

To analyze intestinal permeability to casein, blood was collected from CM-sensitized (1 mg/g plus CT) or control mice 3 hours before and 30 to 40 minutes after intragastric challenge with CM. Sera were prepared and stored at −80° C. Levels of immunologically active casein in serum were measured by inhibition ELISA as previously described (Sampson et al., J. Pediatr. 118:520-5, 1991). Briefly, Immulon II 96-well plates were coated with 0.1 μg/mL of casein in coating buffer (Sigma). After overnight incubation at 4° C. plates were washed with 0.002 mol/L imizadole/0.02% Tween 20 and blocked with 0.07% ovalbumin at RT for 1 hour. Serum samples (1:20 dilution) or casein standards (8 dilutions from 30 μg/mL to 0.1 μg/mL) were incubated with rabbit anti-casein (1:150,000 dilution) antisera (Ross Laboratories) at 37° C. for 2 hours and were then added to the plates (100 mL/well). After incubation for 1 hour at RT, plates were washed. One hundred microliters of horseradish peroxidase-labeled goat anti-rabbit IgG (1:500 dilution; Sigma) was added and incubated for 1 hour at RT. The plates were subsequently washed, and TMB microwell peroxidase substrate (KPL, Gaithersburg, Md.) was added and incubated for 15 minutes at RT. The reaction was stopped by the addition of TMB One Component Stop Solution (KPL) and read at 450 nm. The casein concentrations were determined by comparison with a standard curve.

HISTOLOGY:

Mast cell degranulation during systemic anaphylaxis was assessed by examination of ear samples collected immediately after anaphylaxis-related death or 40 minutes after challenge from surviving mice as previously described (Snider et al., J. ImmunoL. 153:647-57, 1994). Tissues were fixed in 4% phosphate-buffered formaldehyde (pH 7.2), and 5 μm paraffin sections were stained with toluidine blue or Giemsa stain. A degranulated mast cell was defined as a toluidine- or Giemsa-positive cell with 5 or more distinct stained granules completely outside of the cell. One section from each of 3 sites of each mouse ear was examined by light microscopy at 400×magnification by an observer unaware of their identities. Two hundred to 400 mast cells were classified for each ear sample. For assessment of intestinal mast cell degranulation, jejunal samples were fixed in Carnoy's solution and stained with toluidine blue or Giemsa.

For assessment of pathologic alterations, jejunum and lung samples were fixed in neutral-buffered formaldehyde and embedded in paraffin. Five-micrometer sections were stained with hematoxylin and eosin (H and E) and periodic acid-Schiff (PAS) reagent.

Mice were tested for immediate active cutaneous hypersensitivity (IACH) reactions by intradermal skin test 6 weeks after the initial sensitization with CM (1 mg/g plus CT), as previously described with a slight modificati on (Saloga et al., J. Clin. Invest. 91:133-40, 1993; Hsu et al., Clin. Exp. Allergy 26:1329-37, 1996). Briefly under anesthesia the skin of the belly was shaved 1 day before the test. For each skin test, 50 μL of CMP (4 mg/mL) was injected intradermally with a 30-gauge needle while the skin was stretched taut. Antigen concentrations were determined by serial titration to produce consistent wheal reactions. PBS was used as a negative control. The wheal reactions were assessed 30 minutes after intradermal injection with CM. A reaction was scored as positive if the wheal diameter was greater than 3 mm in any direction. Evaluations of wheat formation were carried out in a blinded fashion.

Quantitation of Cytokine Proteins:

Spleens were removed from mice allergic to CM after challenge. Cells were isolated and suspended in complete culture medium (RPM1-1640 plus 10% fetal bovine serum, 1% penicillin/streptomycin, and 1% glutamine). Cell suspensions were cultured in 24-well plates (2×10⁶/well/mL) in the presence or absence of CMP (50 μg/mL) or Concanavalin A (Con A; 2 μg/mL). The supernatants were collected after 72 hours of culture. Levels of IFN-γ, IL-4, and IL-5 were determined by ELISA, according to the manufacturer's instructions (Pharmigen) and as previously described (Li et al., J. Immunol. 157:3216-9, 1996; Li et al., J. Immunol. 160:1378-84, 1998).

Statistical Analysis:

Statistical significance (P<0.05) was determined by t test, ANOVA, or Mann-Whitney U test (rank-sum test). All statistical analyses were performed with GraphPad Prism (GraphPad Prism Software, Inc. San Diego, Calif.).

Results

Cm-Specific Ige Responses After Intragastric Cm Sensitization:

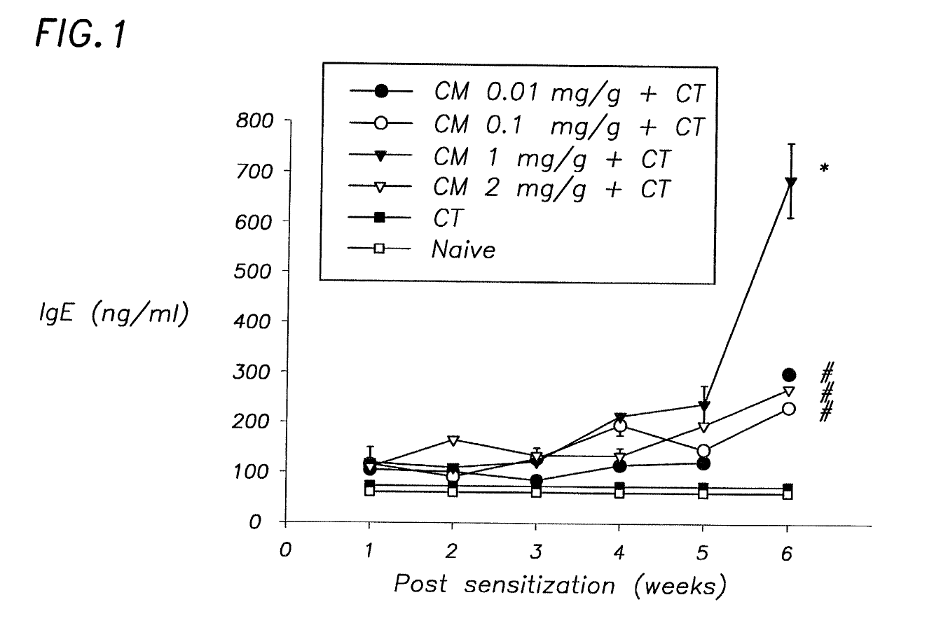

To investigate the kinetics of IgE production in the development of CMH, serum CM-specific IgE was monitored weekly by ELISA. Mice sensitized with the medium dose (1 mg/g) of CM plus CT developed significant (P>0.01) increases in antigen-specific IgE by 3 weeks, which peaked at 6 weeks after the initial sensitization (FIG. 1). Significantly lower levels of antigen-specific IgE were induced by both a higher dose (2 mg/g) and lower doses (0.01, 0.1 mg) of CM plus CT.

Characterization of Systemic Anaphylaxis After Challenge:

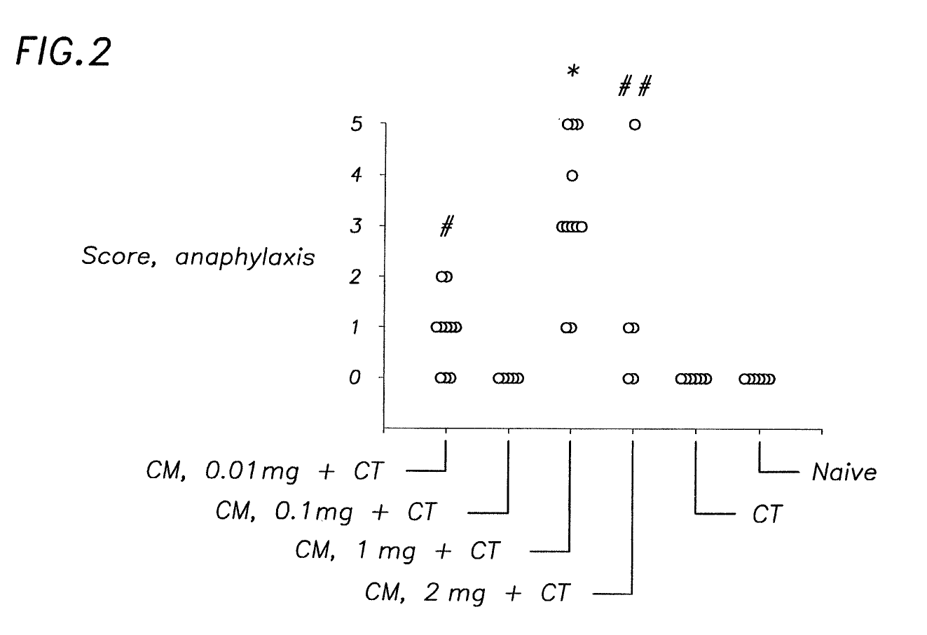

Six weeks after initial sensitization (the time of peak IgE response), the mice were challenged intragastrically with CM. Systemic anaphylactic symptoms were evident within 15 to 30 minutes. The severity of anaphylaxis was scored as indicated above. Consistent with the IgE responses, the most severe reactions were also observed in mice sensitized with the medium dose (1 mg/g) of CM plus CT (FIG. 2). Mice sensitized with the higher and lower doses showed weaker reactions, indicating that the severity of anaphylaxis in this model was associated with the concentration of CM-specific IgE. CT sham-sensitized mice and naive mice showed no anaphylactic reactions after CM challenge. These findings demonstrate that the antigen dose influences the intensity of response to oral sensitization and challenge. Taken together, we concluded that sensitization with CM at the dose of 1 mg/g body weight was optimal, and this dose was used in the remainder of the studies.

Vascular Leakage After Challenge of Sensitized Mice:

Increased vascular permeability, induced by vasoactive mediators such as histamine, is a hallmark of systemic anaphylaxis. Extensive Evan's blue dye extravasation was evident in footpads of CM-sensitized mice, but not CT sham-sensitized mice, after oral challenge (data not shown).

Elevated Plasma Histamine Level After Challenge of Sensitized Mice:

Plasma histamine levels were significantly increased in CM-sensitized (1 mg/g plus CT) mice (4144±1244 nmol/L) after challenge when compared with CT sham-sensitized (661±72 nmol/L) and naive mice (525±84 nmol/L). These results suggest that histamine is one of the major mediators involved in the anaphylaxis in this model.

Increased Mast Cell Degranulation After Challenge of Sensitized Mice:

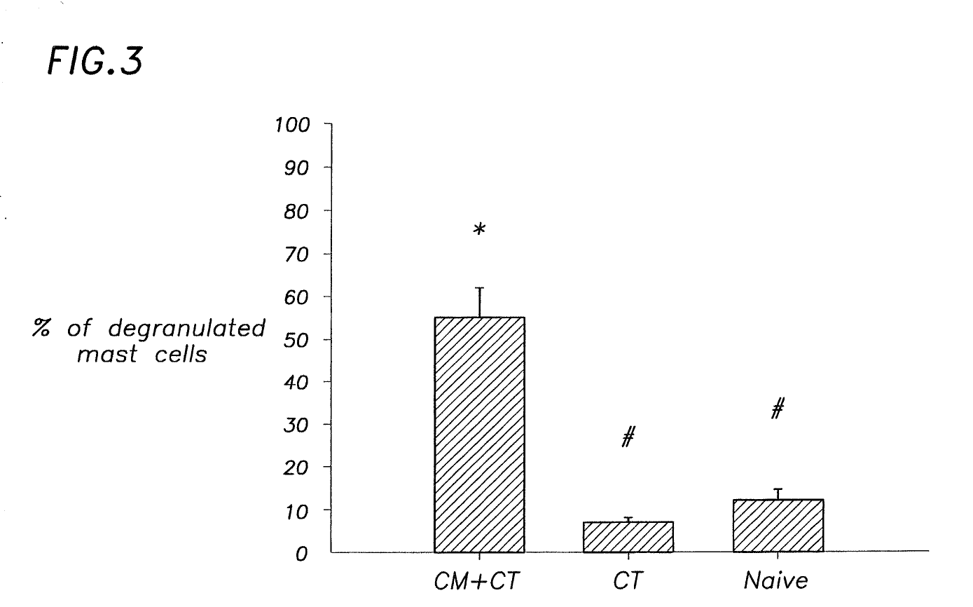

Histologic analysis of mouse ear tissue showed many degranulated mast cells in CM-sensitized and challenged mice, but not control mice (data not shown). The percentage of degranulated mast cells was approximately 9 times greater than that in the PCT sham-sensitized group (FIG. 3). These results were consistent with the findings of elevated levels of plasma histamine after challenge of CM-sensitized mice, demonstrating that mast cell degranulation and consequent histamine release are involved in the induction of systemic anaphylaxis in this model.

Pca Reactions:

Because antigen-specific IgE levels were associated with the severity of anaphylaxis, we hypothesized that IgE, and not IgG1, was responsible for the induction of CMH. To confirm this possibility, PCA testing was performed. Injection PCA reactions, which were eliminated by heat inactivation of immune sera (Table I). These results demonstrate that IgE is the reaginic antibody in this model.

TABLE I
PCA Reactions after injection of heated or unheated immune sera

| DONOR | | | POSITIVE | |
|---|---|---|---|---|
| IMMUNI- | HEAT | DIAMETER (MM) | REACTION | |
| ZATION | INACTIVATION | MEAN ± SEM | N/TOTAL | % |
| CM + CT | — | 8.87 ± 1.14* | 8/8 | 100 |
| CM ± CT | + | 0.58 ± 0.42 | 0/6 | 0 |
| Naive | — | 0.7 ± 0.37 | 0/5 | 0 |

CM-immune sera were obtained from mice sensitized intragastrically with CM (1 mg/g plus CT). Naïve C3H/HeJ mice (n = 5 to 8) received heated or nonheated CM immune sera or naïve mouse sera followed by CMP/Evan's blue dye administration. PCA reactions were assessed 30 minutes later. A reaction was scored as positive if the bluing of the skin at the injection sites was greater than 3 mm in diameter in any direction.

Characterization of Intestinal Reactions:

Increased intestinal permeability after intragastric CM challenge. Altered permeability was assessed in 2 ways: increased mucosal permeability by measurement of serum casein levels and increased intestinal vascular permeability by Evan's blue dye extravasation. Before intragastric challenge with CM, serum casein levels were comparable in CM-sensitized mice (41±20 ng/mL) and in CT control mice (42±12 ng/mL). However, 30 to 40 minutes after challenge, levels of serum casein in CM-sensitized mice (7890±256 ng/mL) undergoing anaphylaxis were significantly higher than those of the control mice (205±23 ng/ML), demonstrating that increased mucosal permeability is a characteristic of this model. Intestines from CM-sensitized mice challenged intragastrically and injected with Evan's blue exhibited dark blue discoloration, whereas naive mice receiving the same antigen/dye administration did not. These results indicate that mucosal and vascular permeability are increased in intestines in this model of milk allergy.

Histologic Analysis of Intestine:

Histologic examination of the small intestines revealed marked vascular congestion and edema of the lamina propria and, in some areas, sloughing of enterocytes at the tips of the villi (data not shown). The histologic appearance was essentially the same as that described in intestinal anaphylaxis in rats (D'Inca et al., Int. Arch. Allergy Appl. Immunol. 91:270-7, 1990; Levine et al., Int. Arch. Allergy Immunol. 115:312-5, 1998). Only a small number of mast cells were observed in the intestines of normal and allergic mice, and most of these were scattered within the serosa. Mast cells were not present within villi and were rarely observed at the base of the crypts. This finding is consistent with prior histochemical and immunohistochemical studies of normal mouse intestines (Carroll et al., Int. Arch. Allergy Appl. Immunol. 74:311-7, 1984; Scudamore et al., Am. J. Pathol 150:1661-1672, 1997). In contrast to the significant numbers of mast cells detected in skin of the same animals, the small numbers of intestinal mast cells precluded analysis of anaphylaxis-induced degranulation.

Characterization of Pulmonary Responses:

We observed that CM-induced immediate reactions in this model were frequently accompanied by respiratory symptoms, such as wheezing and labored respiration. Histologic examination revealed that lungs from these animals were markedly inflamed and contained large numbers of perivascular and peribronchial lymphocytes, monocytes, and eosinophils when compared with control mice (data not shown). Increased numbers of PAS-positive goblet cells were present in bronchi and bronchioles. In some instances the bronchial lumen appeared to be filled with mucus. These lungs exhibited essentially the same appearance as lungs from mice sensitized intraperitoneally and challenged by the intratracheal route (Li et al., J. Immunol., 160:1378-84, 1998; Gavett et al., Am. J. Physiol. 272:L253-61, 1997).

Induction Of Iach After Oral Cm Challenge in Sensitized Mice:

It has been demonstrated that IACH reactions are associated with IgE-induced mast cell degranulation. Thus the IACH has been used for the rapid evaluation of immediate allergic reactions (Saloga et al., J. Clin. Invest. 91:133-40, 1993; Hamelmann et al., J. Exp. Med. 183:1719-29, 1996). Because the first sign of reactions after intragastric challenge was scratching in most of the mice, we performed skin tests at the time of challenge to characterize the skin reactions. Five of 7 (71.4%) CM-sensitized mice experienced IACH-positive reactions after intradermal CMP injection. In contrast, IACH reactions were not induced in CM-sensitized mice after intradermal injection of PBS or in naive mice after intradermal injection of CMP.

Increased TH2—Type Cytokine Responses:

To determine the role of T cells and cytokines in the development of CMA, we examined the production of cytokines by spleen cells from mice allergic to CM stimulated in vitro with CMP. After 72 hours in culture. IL-4 and IL-5 levels were significantly (P<0.001) increased in CMP-stimulated cultures (44 and 68 pg/mL, respectively) when compared with unstimulated cells (undetectable). In contrast, IFN-γ levels in CM-stimulated and unstimulated spleen cells (10 and 14 pg/mL, respectively) were essentially the same (P>0.5).

Skin Disorder:

When the sensitized mice were fed chow containing low levels of milk antigen, the mice developed a skin disorder. The skin disorder included loss of hair. The skin disorder resolved upon feeding the mice milk-free chow. Maintaining the mice on chow containing low levels of the sensitizing antigen may provide for an animal model of skin disorders.

### Example 2

### A Murine Model of Peanut Anaphylaxis

Introduction

This Example describes the development of a mouse model system for anaphylactic peanut (PN) allergy. This system may be employed to identify and characterize chemical compounds capable of desensitizing and/or vaccinating individuals with peanut allergy.

Materials and Methods

Mice and Reagents:

Female C3H/HeJ mice, 5 weeks (wk) of age were purchased from the Jackson Laboratory (Bar Harbor, ME) and maintained on PN-free chow, under specific pathogen-free conditions. Standard guidelines (Institute of Laboratory Animal Resources Commission of Life Sciences NRC. Guide for the Care and Use of Laboratory Animals. National Academy Press, 1996) for the care and use of animals were followed.

Freshly ground whole PN (PN) was employed as antigen (Ag). Crude PN extract, Ara h1 and Ara h 2 were prepared as described previously (Burks et al. “Identification of soy protein allergens in patients with atopic dermatitis and positive soy challenges; determination of change in allergenicity after heating or enzyme digestion” Adv. Exp. Med. Biol. 289:295-307, 1991; Burks et al. “Identification and characterization of a second major peanut allergen, Ara h II, with use of the sera of patients with atopic dermatitis and positive peanut challenge” J. Allergy Clin. Immunol. 90:962-969, 1992). Cholera toxin was purchased from List Biological Laboratories, Inc (Campbell, Calif.). Concanavalin A (Con A), Dinitrophenyl-albumin (DNP-albumin) were purchased from Sigma (St. Louis, Mo.). Antibodies for ELISAs were purchased from the Binding Site Inc. or PharMingen (San Diego, Calif.).

Intragastric Sensitization and Challenge:

Mice were sensitized by intragastric gavage (ig) with 5 mg (equivalent to 1 mg of PN protein, low dose), or 25 mg (equivalent to 5 mg of PN protein, high dose) per mouse of ground whole PN together with 10 mg/mouse of CT on day 0 and again on day 7. Three weeks following the initial sensitization, mice were fasted over night and challenged by ig with crude PN extract, 10 mg/mouse divided in 2 doses at 30-40 min. intervals. CT sham sensitized mice and naive mice were challenged in the same manner. Mice surviving the first challenge were re-challenged at wk 5.

Assessment of Hypersensitivity Reactions:

Anaphylactic symptoms were evaluated 30-40 minutes following the second challenge dose utilizing a scoring system, modified slightly from previous reports (Li et al. “A Murine Model of IgE Mediated Cow Milk Hypersensitivity” J. Allergy Clin. Immunol. 103:206-214, 1999; Poulsen et al. “Effect of homogenization and pasteurization on the allergenicity of bovine milk analysed by a murine anaphylactic shock model” Clin. Allergy 17:449-458, 1987; McCaskill et al. “Anaphylaxis following intranasal challenge of mice sensitized with ovalbumin” Immunology 51:669-677, 1984) 0—no symptoms; 1—scratching and rubbing around the nose and head; 2—puffiness around the eyes and mouth, diarrhea, pilar erecti, reduced activity, and/or decreased activity with increased respiratory rate; 3—wheezing, labored respiration, cyanosis around the mouth and the tail; 4—no activity after prodding, or tremor and convulsion; 5—death.

Measurement of Plasma Histamine Levels:

To determine plasma histamine levels, blood was collected 30 minutes after the second ig challenge. Plasma was prepared as previously described (Li et al. “Strain-dependent induction of allergic sensitization caused by peanut allergen DNA immunization in mice” J. Immunol. 162:3045-3052, 1999; Li et al. “A Murine Model of IgE Mediated Cow Milk Hypersensitivity” J. Allergy Clin. Immunol 103:206-214, 1999) and stored at −80° C. until analyzed. Histamine levels were determined using an enzyme immunoassay kit (ImmunoTECH Inc., ME), as described by the manufacturer.

Measurement of Pn-Specific Ige:

Tail vein blood was obtained at weekly intervals following initial sensitization. Sera were collected and stored at −80° C. Levels of PN-specific IgE were measured by ELISA. Briefly, Immulon II 96-well plates (Dynatech Laboratories, Inc., Chantilly, Va.) were coated with 2 mg/ml CPE in coating buffer, pH 9.6 (Sigma, St. Louis, Mo.). All the steps thereafter followed the same protocol described previously (Li et al. “Strain-dependent induction of allergic sensitization caused by peanut allergen DNA immunization in mice” J. Immunol. 162:3045-3052, 1999). All analyses were performed in duplicate and coefficient of variation (CV) >10% were repeated to ensure a high degree of precision.

HISTOLOGY:

Mast cell degranulation during systemic anaphylaxis was assessed by examination of ear samples collected immediately after anaphylactic death or 40 min. after challenge from surviving mice as previously described (Li et al. “Strain-dependent induction of allergic sensitization caused by peanut allergen DNA immunization in mice” J. Immunol. 162:3045-3052, 1999; Snider et al. “Production of IgE antibody and allergic sensitization of intestinal and peripheral tissues after oral immunization with protein Ag and cholera toxin” J. Immunol. 153:647-657, 1994). Tissues were fixed in 10% neutral buffered formalin and 5-mm paraffin sections were stained with toluidine blue or Giemsa. Sections from three sites of each mouse ear were examined by light microscopy at 400×by an observer unaware of their identities. A degranulated mast cell was defined as a toluidine blue or Giemsa-positive cell with five or more distinct stained granules completely outside of the cell. Four hundred mast cells in each ear sample were classified.

Proliferation Assays:

Spleens were removed from PN sensitized and naive mice after re-challenge at wk 5. Spleen cells were isolated and suspended in complete culture medium (RPMI 1640 plus 10% fetal bovine serum, 1% penicillin/streptomycin, and 1% glutamine). Spleen cells (1×10⁶/well in 0.2 ml) were incubated in triplicate cultures in microwell plates in the presence or absence of crude PN extract, Ara h 1, or Ara h 2 (10, 50 μg/ml). Cells stimulated with Con A (2 μg/ml) were used as a positive control. Six days later, the cultures were pulsed for 18-hr with 1 μCi per well of ³H-thymidine. The cells were harvested and the incorporated radioactivity was counted in a β-scintillation counter. The results were expressed as counts per minute (cpm).

Two-Dimensional Gel Electrophoresis and Immunoblotting:

Two-dimensional gel electrophoresis was employed to separate PN proteins using previously described methods with slight modifications (Burks et al. “Identification and characterization of a second major peanut allergen, Ara h II, with use of the sera of patients with atopic dermatitis and positive peanut challenge” J. Allergy Clin. Immunol. 90:962-969, 1992; O'Farrell et al. “High resolution two-dimensional electrophoresis of basic as well as acidic proteins” Cell 12:1133-1141, 1977; Hochstrasser et al. “Methods for increasing the resolution of two-dimensional protein electrophoresis” Anal. Biochem. 173:424-435, 1988). The first dimension consisted of an isoelectric focusing gel in glass tubing. After making the gel mixture with a pH gradient of 3.5-10 (BioRad Laboratories) 200 mg samples were loaded and focused with a BioRad Protean II xi 2-D cell at 200 V for 2 hours, 500 V for 2 hours and 800 V overnight. The second dimension gel, sodium dodecyl sulphate-polyacrylamide gel (SDS-PAGE), employed an 18% polyacrylamide separating and a 4% stacking gel as previously described (Burks et al. “Identification and characterization of a second major peanut allergen, Ara h II, with use of the sera of patients with atopic dermatitis and positive peanut challenge” J. Allergy Clin. Immunol. 90:962-969, 1992; Laemmli “Cleavage of structural proteins during the assembly of the head of bacteriophage T4” Nature 227:680-685, 1970). Electrophoresis was performed for 18 hours at 25 mA per 14 cm by 12 cm gel with a set limit of 150 V in a Hoefer Apparatus (Pharmacia Biotech).

Proteins were transferred from the separating gel to a 0.22 mm nitrocellulose membrane in a Tris-Glycine buffer containing 20% methanol. The procedure was performed in a Hoefer transfer unit for 14 hours at 100 mA. To assure proper protein separation and quality of transfer, one nitrocellulose membrane from each pair was stained with Amido-Black, while both polyacrylamide gels were stained with Coomassie Brilliant Blue.

After removal from the transblot apparatus, the nitrocellulose membranes were placed in blocking solution (PBS containing 0.5% gelatin, 0.05% Tween and 0.001% thimerosal) overnight at RT on a rocking platform. The nitrocellulose blot was then washed three times with PBS containing 0.05% Tween (PBST) and incubated with pooled sera from highly sensitive PN-allergic patients [1:10 dilution in a blocking solution] for two hours at RT. After rinsing and washing four times with PBST, alkaline phosphatase-conjugated goat anti-human IgE (KPL, 0.5 mg/ml) was added and incubated at RT for 2 hours. After rinsing and washing with PBST four times, the blot was developed with BCIP/NBT Phosphatase Substrate System (KPL) for 5 min. The reaction was stopped by washing the nitrocellulose membrane with distilled water and the blot was air-dried.

For characterization of mouse IgE antibody binding to allergenic PN proteins, the nitrocellulose blot was prepared as above. The blot was incubated with pooled sera from PN-sensitive mice [1:10 dilution] overnight at RT, followed by extensive washes with PBST and another overnight incubation in 0.75 mg/ml sheep anti-mouse IgE (The Binding Site, UK). The blot was then washed 4 times and 0.3 mg/ml horseradish peroxidase conjugated donkey anti-sheep IgG (The Binding Site, UK) was added. After 2 hours incubation at RT, the blot was washed and developed with TMB Membrane Substrate Three Component System (KPL) for 15 min., washed with distilled water, and air-dried.

Mapping of Mouse IgE Binding Epitopes:

The 157 amino acids comprising Ara h 2 were synthesized as 73 overlapping peptides. Each peptide was 13 amino acids long and offset from the adjacent peptide by 2 amino acids. Individual peptides were synthesized on a derivatized cellulose membrane by Genosys Biotechnologies (Houston, Tex.). The cellulose membrane containing the synthesized peptides was washed with Tris-buffered saline containing 1% Tween (TBST) and then incubated with blocking solution of TBST containing 1% BSA overnight at 4° C. After blocking, the membrane was incubated for 15 h at 4° C. with pooled sera from PN-sensitized mice (3600 ng/ml IgE) that had been diluted 1:10 in a solution containing TBST and 1% bovine serina albumin. Primary antibody was detected with I²⁵I-labeled anti-IgE antibody. The secondary antibody is a rat anti-mouse IgE monoclonal antibody (Southern Biotechnology Associates; Birmingham, Ala.) iodinated by DiaMed, Inc. (Windham, Me.) [¹²⁵I label -18.6 μCi/mg specific activity]. The membrane was exposed to X-ray film and then densitometric scans were made of the autoradiographs to determine the relative amounts of IgE bound to each peptide.

Results

Systemic Anaphylactic Reactions Following Intragastric Challenge:

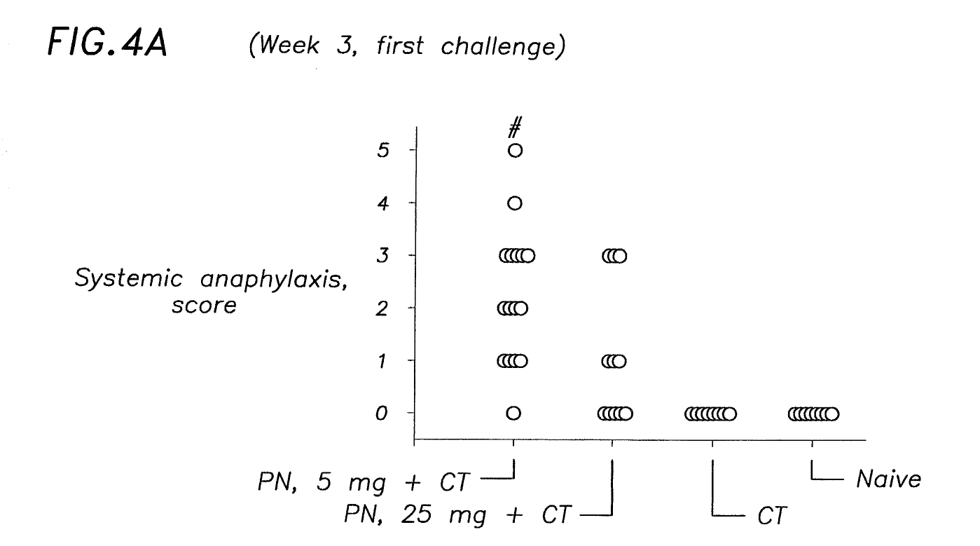

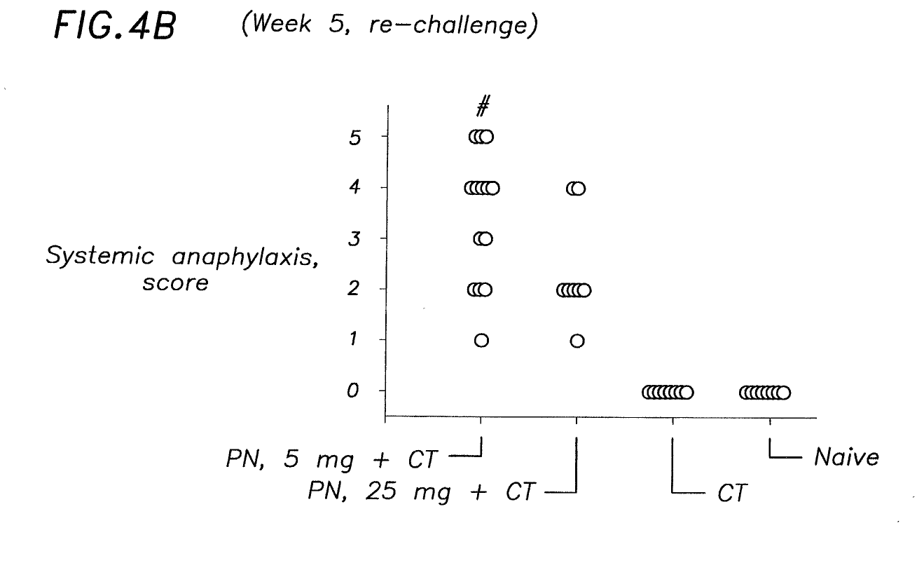

Three weeks following the initial sensitization, mice were fed with crude PN extract by ig at 30-40 min. intervals. Systemic anaphylactic symptoms were evident within 10-15 min following the first dose, and the severity of the anaphylaxis was evaluated 30-40 min. after the second dose. The initial reactions consisted primarily of cutaneous reactions with puffiness around the eyes and mouth, and/or diarrhea followed by respiratory reactions such as wheezing and labored respiration. The most severe reactions provoked loss of consciousness and death (FIG. 4A). Mice sensitized with the low dose (5 mg/mouse+CT) of whole PN exhibited more severe reactions than those sensitized with the high dose (25 mg/mouse+CT). Fatal or near-fatal anaphylaxis occurred in 12.5% of low dose sensitized mice but in none of the high dose sensitized mice. Sham-sensitized and naive mice did not show any symptoms of anaphylaxis. Two weeks following the first challenge, the surviving mice were re-challenged. Systemic anaphylactic reactions were again provoked, and were more severe than those induced by the first challenge at wk 3 with both 5 mg (symptom score wk 5 verses (vs) wk 3, p=0.009), and 25 mg PN (symptom score wk 5 vs wk 3, p=0.03). However, as noted during the challenge at wk 3, symptom scores at wk 5 (re-challenge) were significantly higher in the group sensitized with 5 mg of peanut, with a 21% mortality rate, than those in the group sensitized with 25 mg (p<0.05). These results showed that the initial sensitizing dose determined the intensity of the hypersensitivity reactions. We concluded that sensitization with PN at the dose of 5 mg/mouse (low dose) was optimal and this dose was used for subsequent studies.

Increased Mast Cell Degranulation And Histamine Release Following Ig-Challenge:

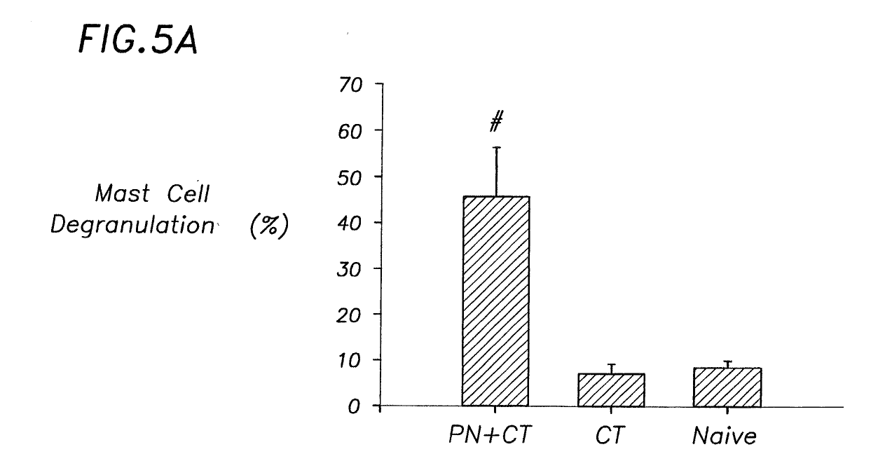

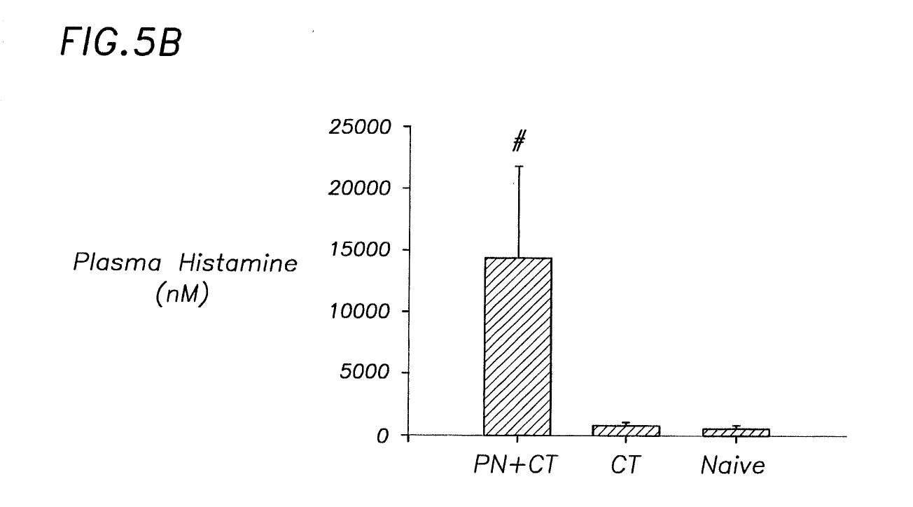

The percentage of degranulated mast cells in ear tissues were significantly greater in PN sensitized mice than in controls following PN-challenge (FIG. 5A). Consistent with this finding, plasma histamine levels also were significantly increased in PN-sensitized mice compared with CT sham-sensitized and naive mice (FIG. 5B). These results suggest that histamine [and probably other mediators] released from mast cells contributed to the symptoms of PN-induced anaphylaxis.

Increased Pn-Specific Ige Following Pn-Sensitization and Challenge:

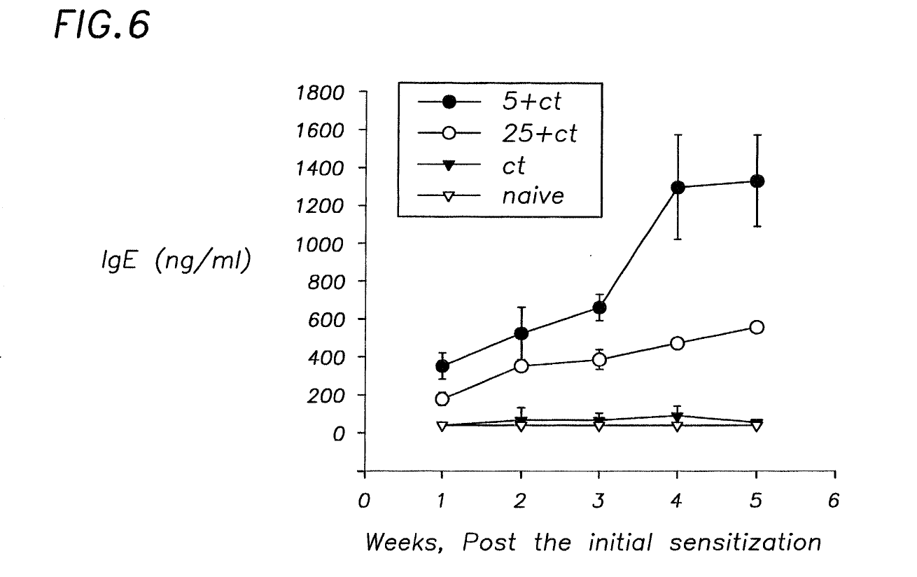

To determine PN-specific IgE responses in this model, sera from each group of mice were obtained weekly following ig sensitization and challenge. PN-specific IgE concentrations increased significantly from wk 1 through wk 5 in mice sensitized with low dose PN (5 mg/mouse), and from wk 2 through wk 5 in mice sensitized with high dose PN (25 mg/mouse) (FIG. 6). PN-specific IgE levels were significantly higher in the low dose group compared to the high dose group at both wk 3 (initial challenge) and wk 5 (re-challenge), suggesting an association between IgE levels and severity of anaphylactic reactions. PN-specific IgG1 levels increased significantly in both groups, but did not differ in the high or low dose sensitization groups (wk 5, 57.6 vs 57 μg/ml), suggesting that IgG1 was not associated with PN-hypersensitivity reactions in this model.

Pca Reactions:

To confirm that IgE was responsible for the induction of PN hypersensitivity, and to rule-out IgG1-mediated anaphylaxis, PCA testing was performed. Injection of sera from PN-allergic, but not normal mice, induced PCA reactions. These reactions were eliminated by heat (56° C. for 3 h) inactivation of immune sera (Table II), indicating that IgE is the reagenic Ab in this model.

TABLE II
PCA reactions following injection of heated or non-heated immune sera

| | Heat | | Positive | |
|---|---|---|---|---|
| Donor | inactivation | Diameter (mm) | reaction | |
| (immunization, ig) | (56° C. 3 h) | (Mean ± SE) | n/total | % |
| PN + CT | − | 8.28 ± 1.20* | 7/7 | 100 |
| PN + CT | + | 1.16 ± 0.40 | 0/6 | 0 |
| Naive | − | 1.20 ± 0.37 | 0/5 | 0 |

Naïve mice (n = 5-8) were injected with heated or non-heated PN immune sera followed by iv Ag/Evan's blue dye injection. PCA reactions were assessed 30 min. later. A reaction was scored as positive if the bluing of the skin at the injection sites was >3 mm in diameter in any direction.
*P < 0.001 vs. heated immune sera.

T-Cell Proliferative Responses to Whole Pn and the Major Pn Allergen Ara H 1 and Ara H 2 Resemble Those of Human Pna:

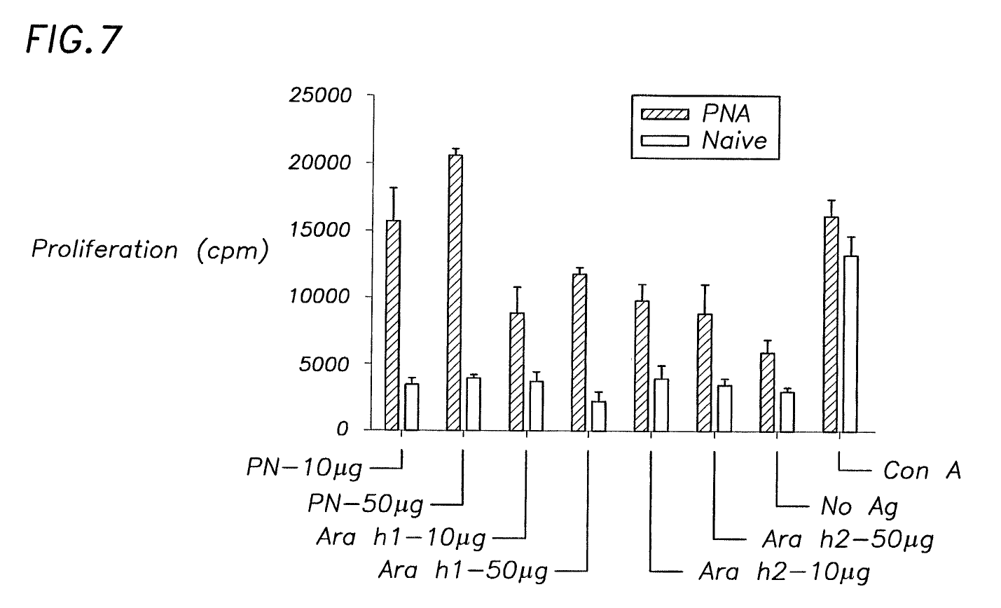

To characterize T cell responses to whole PN, or major PN allergens in this model, spleen cells from PN-allergic mice or naive mice were cultured with crude PN extract, Ara h 1, or Ara h 2. Although cells from both PN-allergic mice and naive mice showed significant proliferative responses to Con A stimulation, cells from PN-allergic mice, but not from naive mice, exhibited significant proliferative responses to crude PN, Ara h 1, and Ara h 2 stimulation (FIG. 7). These results demonstrated that the T cell responses to PN and the major PN allergens were similar to those observed in PN allergic patients (Dorion et al. “The production of interferon-gamma in response to a major peanut allergy, Ara h II correlates with serum levels of IgE anti-Ara h II” J. Allergy Clin. Immunol. 93:93-99, 1994).

B-Cell Ig Responses to the Major Pn Allergens, Ara H 1 AND Ara H 2 Resemble Those of Human Pna:

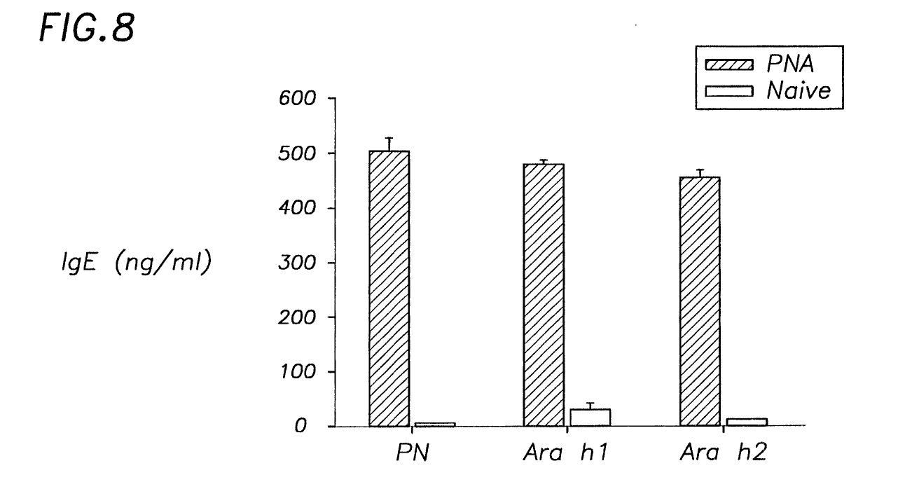

To determine B cell responses to the major PN allergens, IgE antibodies against Ara h I and Ara h 2 were measured in pooled sera of PN-sensitized mice and naive mice. Both Ara h 1- and Ara h 2-specific IgE were present in the sera of PN-allergic mice (FIG. 8). These results demonstrated that B cell IgE responses to PN allergens in this model resemble those in human PNA.

Comparison of Pn-Allergic Mouse and Pn-Allergic Human Ige Antibody Binding to the Major Pn Allergen, ARA H 2:

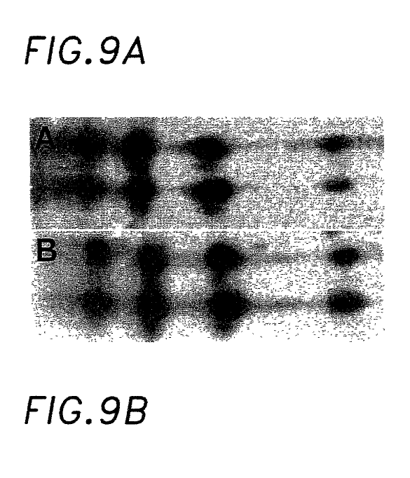

Following the detection of anti-Ara h 1 and anti-Ara h 2—specific IgE antibodies in pooled sera of PN-allergic mice, we compared PN-allergic mouse and human IgE antibody binding to the major PN allergen Ara h 2 fractions by employing two-dimensional gel electrophoresis and immunoblotting. FIG. 9A shows that human IgE recognizes 8 Ara h 2 isoforms which have been previously characterized (Sampson et al., manuscript is in preparation). FIG. 9B shows that IgE from PN-sensitized mice recognized the same Ara h 2 isoforms as human IgE.

Arah 2 Ige Binding Epitopes are Similar in Mouse and Man:

The finding that mouse IgE recognized the same Ara h 2 isoforms as human IgE suggested that mouse IgE and human IgE might bind to similar Ara h 2 epitopes. To confirm this possibility, we mapped mouse Ara h 2 IgE-binding epitopes. Seventy-three overlapping peptides representing the amino acid sequence of the Ara h 2 protein were synthesized, as indicated above, to determine which regions were recognized by serum IgE. These peptides were probed with pooled sera from PN-sensitized mice. Table III depicts the 11 IgE-binding epitopes identified. These epitopes were distributed throughout the length of the Ara h 2 protein with 9/11 in essentially the same regions as those previously identified as human IgE-binding epitopes (Stanley et al. “Identification and mutational analysis of the immunodominant IgE binding epitopes of the major peanut allergen Ara h 2” Arch. Biochem. Biophys. 342:244-253, 1997).

TABLE III
Comparison of mouse and patient IgE antibody binding to
Ara h 2 epitopes

| Mouse IgE | Human IgE | Ara h 2 | Percentage IgE |
|---|---|---|---|
| Binding Epitope | Binding Epitope | Position | Binding (M) |
| LFLLAAH(1) | HASARQQWEL(1) | M9-15 | 10.8 |
| | | H15-24 | |
| RQQWELQGDRR(2) | QWELQGDR(2) | M19-29 | 10.9 |
| | | H21-28 | |
| RCQSQLERA(3) | DRRCQSQLER(3) | M29-37 | 4.4 |
| | | H27-36 | |
| | LRPCEQHLMQ(4) | H39-48 | |
| DEDSYERDP(4) | KIQRDEDS(5) | M53-61 | 35.0 |
| | | H49-56 | |
| YERDPYSPS(5) | RDPYSP(6) | M57-65 | 14.3 |
| | | H59-64 | |
| YSPSPYD(6) | SQDPYSPS(7) | M69-75 | 7.0 |
| | | H65-72 | |
| QQEQQFK(7) | LQGRQQ(8) | M121-127 | 4.0 |
| | | H117-122 | |
| KRELRNLPQ(8) | KRELRN(9) | M127-135 | 4.1 |
| | | H127-132 | |
| RNLPQQCGL(9) | | M131-139 | 3.1 |
| CGLRAPQ(10) | | M137-143 | 3.1 |
| QRCDLDV(11) | QRCDLDVE(10) | M143-149 | 3.0 |
| | | H143-153 | |

Number in parentheses indicate epitiope number.
M = mouse, H = human.
Human IgE binding epitopes from Stanley et al., Arch. Bioch. Biophys., 342:244-253, 1997.

Since the mouse Ara h 2 IgE-bindinrg epitopes were similar to the human epitopes, we wondered if the same epitopes were also immunodominant. In humans, Ara h 2 amino acid residues 57-74 are considered to be immunodominant because they are recognized by IgE from all PN-sensitive patients. In addition, serum IgE antibodies that recognize this region represent the majority of Ara h 2-specific IgE found in PN-allergic patients (Stanley et al. “Identification and mutational analysis of the immunodominant IgE binding epitopes of the major peanut allergen Ara h 2” Arch. Biochem. Biophys. 342:244-253, 1997). In order to determine which, if any, of the 11 mouse epitopes was immunodominant, the intensity of IgE binding to each peptide was determined as a function of the pool's total IgE binding to all epitopes. The region represented by amino acid residues 53-75 bound 56.3% of the Ara h 2-specific mouse IgE indicating that, as has been observed in humans, this region is also immunodominant in PN-sensitized mice.

### Example 3

### A Murine Model of Peanut Anaphylaxis Using Peanut Antigen Encoding DNA

Introduction

This Example describes the development of a mouse model system for anaphylactic peanut (PN) allergy using gene immunization.

Materials and Methods

Mice and Reagents:

Female C3H/HeSn (H-2^{K}), BALBIc (H-2^{d}), and male AKR (H-2^{k}) mice, 6 wk of age, were purchased from the Jackson Laboratory (Bar Harbor, Me.) and maintained on peanut protein-free chow under specific pathogen-free conditions.

PN and Ara h 2 protein were prepared as previously described (Kopper et al. “Rapid isolation of peanut allergen and their physical and biological characterization” J. Allergy Clin. Immunol 101:S240, 1998). Ara h 2 cDNA was generated as previously described (Stanley et al. “Identification and mutational analysis of the immunodominant IgE binding epitopes of the major peanut allergen Ara h 2” Arch. Biochem. Biophys. 342:244, 1997). Conalbumin (CA), Con A, DNP-BSA, and ovomucoid were purchased from Sigma (St. Louis, Mo.), Abs for ELISAs were purchased from The Binding Site (San Diego, Calif.).

Plasmid Dna Preparation:

The plasmid DNA-based gene construct, pAra h 2, generated by using a TA cloning kit (Invitrogen, San Diego, Calif.). Briefly, PCR-amplified Ara h 2 coding region gene segment with the addition of a Kozak consensus translation condon was ligated into a pCR3.1-Uni expression vector containing CMV promoter. The pOMC was also generated using the same vector, pCR 3.1-Uni, encoding the ovomucoid, a major allergen from egg. The plasmid DNA pcDNA3 (pcDNA) (Invitrogen) was used as a mock DNA control since its backbone is identical to pAra h 2 and pOMC, with the exception of the cloning site. The pDNA was prepared and purified by BioServe (Laurel, Md.), and resuspended in endotoxin-free water.

Dna Immunization and Ag Administration:

Mice were anesthetized by i.p. injection with a mixture of ketamine (45 mg/g) and xylazine (10 mg/g), and each mouse was then injected i.m. with 15 μg of naked pDNA diluted in PBS to a final volume of 50 pl. In the dose-dependent study, mice received one injection (single immunization) or three daily injections, followed by a forth injection 1 wk later (multiple immunization). Control mice received mock DNA (pcDNA), or were untreated. Three weeks after the initial pDNA immunization, mice were injected i.p. with 1 mg/mouse of PN or Ara h 2-purified protein, or an irrelevant Ag, CA.

Measurement of Serum Ag-Specific Abs:

Blood was obtained weekly from each group of mice following the initial pDNA immunization. After centrifugation, the sera were collected and stored at −80° C. until analyzed. The levels of Ag-specified IgE, IgG1, and IgG2a Abs were measured by ELISA, as described previously (Li et al. “Induction of pulmonary allergic responses by antigen-specific Th2 cells” J. Immunol. 160:1378, 1998). Immulon II plates (Dynatech Laboratories, Chantilly, Va.) were coated with 10 μg/ml purified Ara h 2 protein in coating buffer (Sigma). After overnight incubation at 4° C., plates were washed three times with PBS/0.05% Tween-20 and blocked with 1% BSA-PBS for 1 h at 37° C. After three washings, serum samples (⅕ or 1/10 dilutions in 1% BSA-PBS) were added to the plates and incubated overnight at 4° C. Plates were then washed, and 100 μl of goat anti-mouse IgE or IgG1, or IgG2a Abs (0.3 μg/ml) were added to the plates for detection of IgE, IgG1, and IgG2a Abs, respectively. The plates were incubated for 2 h at 37° C. After three washings, 100 μl of donkey anti-goat IgG Ab conjugated with peroxidase (0.3 μg/ml) was added for 1 h at 37° C. Plates were developed with tetramethylbenzidine (TMB) (Bio-Rad, Hercules, Calif.) for 30 min at 22° C., stopped by the addition of 1 N H₂SO₄, and read at 450 nm. The levels of IgE, IgG1, and IgG2a Abs were calculated by comparison with a reference curve generated by using mouse mAbs, anti-DNP IgE, IgG1, and IgG2a (Accurate Scientific, Westbury, N.Y.). All analyses were performed in duplicate and discrepant values (coefficient of variation>10%) were repeated to ensure a high degree of precision. Values less than 4 mg/ml were regarded as undetectable in this assay.

Assessment of Hypersensitivity Responses:

Signs of systemic anaphylaxis became apparent in C3H mice 10 to 15 min following i.p. PN injection and peaked at 20-40 min. Symptoms of tiff anaphylaxis were evaluated by a scoring system 40 min after challenge. This scoring system was modified slightly from previous descriptions (Mccaskill et al. “Anaphylaxis following intranasal challenge of mice with ovalburnin” J. Immunol. 51 :669, 1984; Poulsen et al. “Effect of homogenization and pasteurization on the allergenicity of bovine milk analyzed by a murine anaphylactic shock model” Clin. Allergy 17:449, 1987), and scored as follows: 0, no symptoms; 1, scratching and rubbing around the nose and head; 2, puffiness around the eyes, pilar erecti, reduced activity, and/or decreased activity with increased respiratory rate; 3, wheezing, labored respiration, cyanosis around the mouth and the tail; 4, no activity after prodding, or tremor and convulsion; 5, death.

Detection of Vascular Leakage:

At the time of peanut protein Ag injection, C3H mice from each group received 100 μl of 0.5% Evan's blue dye by tail vein injection, immediately followed by i.p. peanut injection. Thirty to forty minutes after dye/Ag administration the mice's feet were examined for signs of vascular leakage (visible blue color).

Determination of Plasma Histamine Levels:

Five to eight minutes following peanut injection, 0.3-0.5 ml of blood from each mouse was collected into chilled tubes containing 30-40 μl of 7.5% potassium-EDTA. After centrifugation (1500 rpm) for 10 min at 4° C. the plasma was collected and frozen at −80° C. until used. The levels of histamine were determined using an enzyme immunoassay kit (Immunotech, Westbrook, Me.), as described by the manufacturer. The concentration of histamine was calculated by comparison with a standard curve provided by the manufacturer.

Histologic Studies:

Mast cell degranulation during systemic anaphylaxis was assessed by histologic examination of ear tissues. Samples were collected immediately after anaphylaxis-related death or 40 min after challenge from surviving mice. Tissues were fixed in 4% paraformaldehyde, 2.5% glutaraldehyde in 0.1 M sodium cacodylate buffer (pH 7.3), at room temperature for 30 min then stored at 4° C. until processing into 3 μm parafin or glycol methacrylate, toluidine blue-stained sections. A degranulated mast cell was defined as a toluidine-positive cell with five or more distinct stained granules completely outside of the cell (Snider et al. “Production of IgE antibody and allergic sensitization of intestinal and peripheral tissues after oral immunization with protein antigen and cholera toxin” J. Immunol. 143 :647, 1994). One section from each of three sites of each mouse ear was examined by light microscopy at 400×by an observer unaware of their identities. A total of 200-400 mast cells was classified in each ear sample. In some instances, Wright's stained blood smears were also prepared from mice experiencing anaphylactic shock.

Passive Cutaneous Anaphylaxis (Pca) Test:

Sera were obtained from 4 to 6 pAra h 2 multiply immunized C3H mice and pooled. The PCA test was modified slightly from previous descriptions (Saloga et al. J. Clin. Invest. 91:133, 1998; Poulsen et al. “Murine passive cutaneous anaphylaxis test (PCA) for the ‘all or none’ determination of allergenicity of bovine whey protein and peptide” Clinical Allergy 17:75, 1987). Briefly, the abdomens of naive C3H mice were carefully shaved 1 day before i.d. injection of 30 μl of heated (56° C. for 3 h) and unheated undiluted sera. Control mice received equal amounts of pooled sera from mock DNA-immunized mice or an equal amount of PBS. Injections were repeated 24 h later. Three hours after the second injection, mice were injected i.v. with a mixture of 100 μl of 0.5% Evan's blue dye and 1 mg PN protein. Thirty minutes following the dye/PN injection, the mice were scarified, the skin of the belly was inverted, and PCA reactions were examined by visible blue color. A reaction was scored as positive if the bluing of the skin at the injection site was >0.5 cm in diameter.

Quantitation of Cytokines:

Spleens were removed from each group of mice at 3 wk after pDNA immunization. Cells were isolated and suspended in complete culture medium (RPMI 1640 plus 10% FBS, 1% penicillin/streptomycin, and 1% glutamine). Cell suspensions were cultured in 24-well plates (4×10⁶/well/ml) in the presence or absence of PN (50 μg/ml) (Ara h 2 comprises 15-20% of total peanut protein) or Con A (2 μg/ml). The supernatants were collected after 24-, 48-, and 72-h culture. Levels of cytokines, IFN-γ, IL-4, and IL-5 were determined by ELISA, according to the manufacturer's instructions (PharMingen, San Diego, Calif.) and as previously described (Li et al. “Induction of pulmonary allergic responses by antigen-specific Th2 cells” J. Immunol. 160:1378, 1998).

Statistical Analysis:

The statistical significance of the data was determine by ANOVA or test A p value of <0.05 was considered significant.

Results

Isotype Profile of Ag-Specific Abs Induced by Pdna Immunization in C3H Mice:

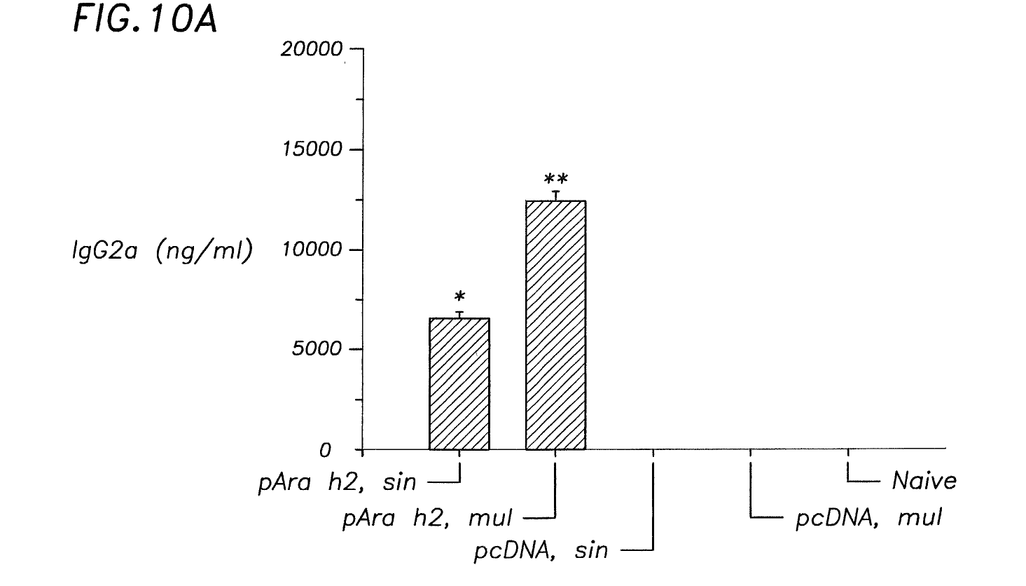

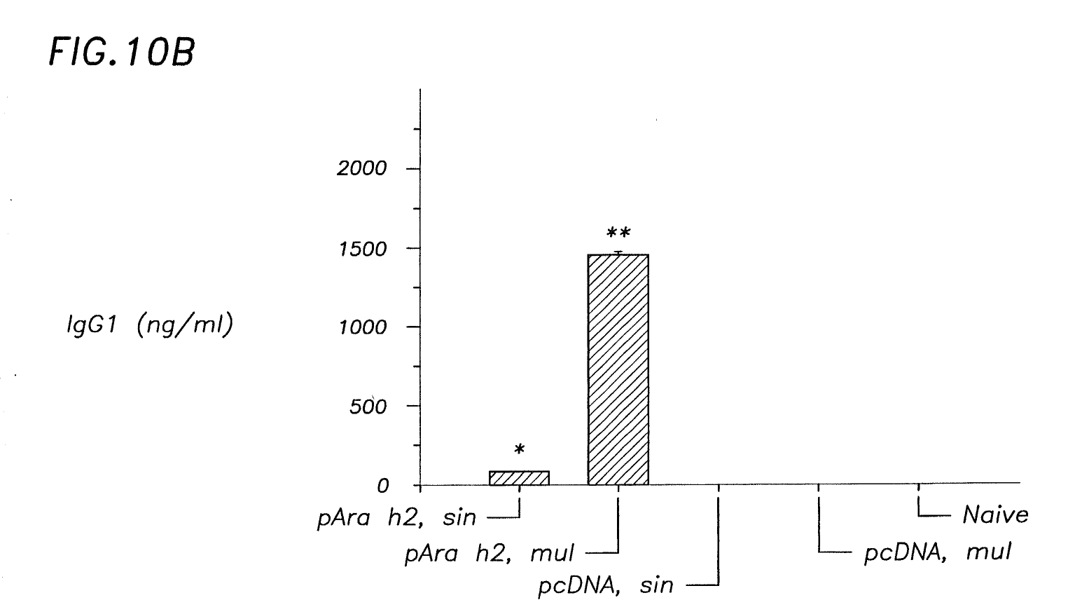

Three weeks after the initial pDNA immunization of C3H mice, significantly increased levels of Ara h 2-specific IgG2a as well as IgG1 were present in pAra h 2-immunized-mice (FIG. 10), but not in pcDNA (mock DNA)-immunized mice. The level of IgG2a was 10-fold higher than IgG1. The dose-dependent study showed that IgG2a in the multiply immunized group was twofold higher than in the single immunized group. The titer of IgG1 in the multiply immunized group was 30-fold higher than that in singly immunized group. No Ara h 2-specific IgE was detectable in either singly or multiply immunized mice (data not shown). In addition, multiple i.d. injections of pAra h 2 produced significant increase of Ara h 2-specific IgG1 (data not shown).

Induction of Anaphylactic Reactions by Pn Injection of Para H 2-Immunized C3H Mice:

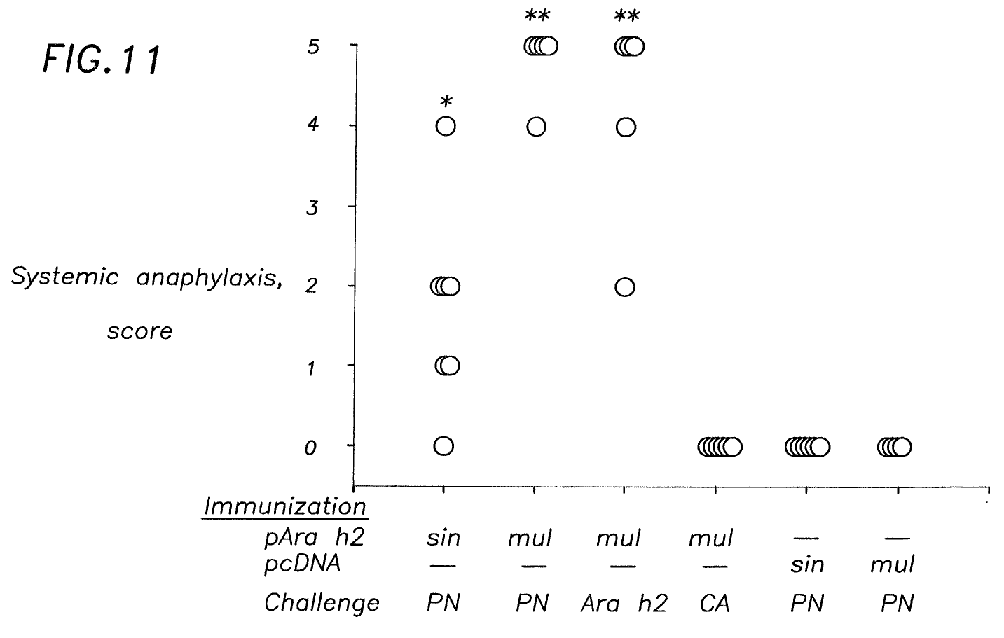

The initial experiment was designed to investigate whether pAra h 2 immunization could prevent peanut-induced hypersensitivity, as reported for different Ags by others (Hsu et al. Nat. Med. 2:540, 1996; Raz et al. Proc. Natl. Acad. Sci. USA 93:5141, 1996). In this study, mice were immunized with pDNA 3 wk before peanut protein Ag sensitization. Surprisingly, i.p. injection of either PN or Ara h 2 protein into the mice immunized with pAra h 2 result in anaphylactic reactions. The severity of the reactions was evaluated and scored as shown in FIG. 11. Anaphylactic reactions in the multiply pAra h 2-immunized group were more severe than in singly immunized mice, with a mortality rate of 60%, indicating an association between the increased level of IgG1 and the severity of the anaphylactic reactions. No anaphylactic reactions were observed in mock DNA-immunized mice following peanut injection or in the pAra h 2immunized mice following injection with an irrelevant Ag CA. Thus, the anaphylactic reactions in this model were Ag specific and dose dependent.

Increased Vascular Permeability Following Pn Injection of Para H 2-Immunized C3H Mice:

Increased vascular permeability is a hallmark of systemic anaphylaxis. To further characterize the anaphylaxis, vascular leakage was assessed by PN/Evan's blue injection. Extensive Evan's blue extravasation was evident in mouse feet of pAra h 2-immunized mice (data not shown). In addition, peripheral blood smears showed extensive platelet aggregation in pAra-h 2-immunized mice following PN administration (data not shown).

Elevated Plasma Histamine Following Pn Injection of Para H 2-Immunized C3)H Mice:

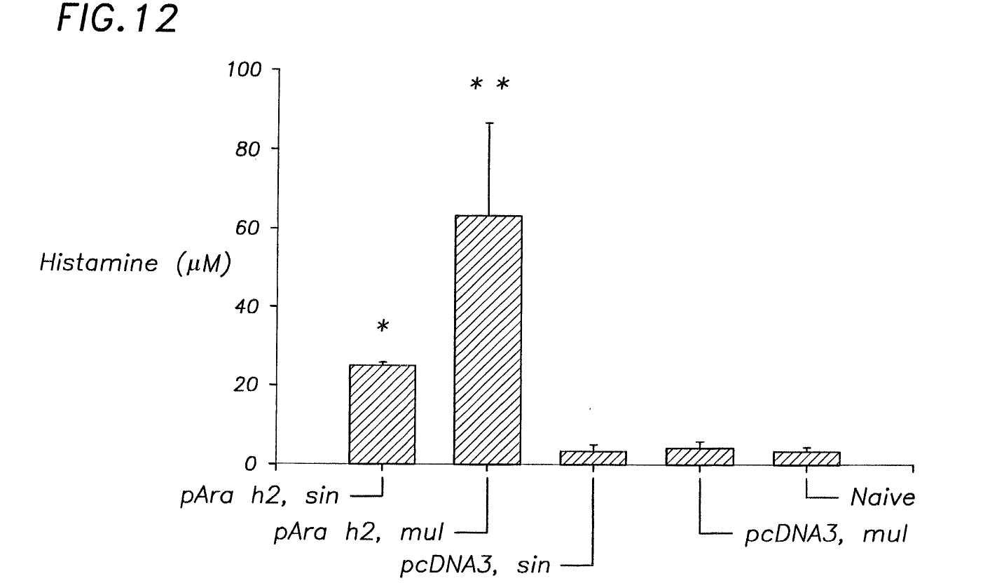

Following PN administration, plasma histamine was increased significantly in the pAra h 2-immunized group when compared with control groups (FIG. 12). Moreover, the histamine levels in pAra h 2 multiply immunized mice were significantly greater than in singly immunized mice. These results indicate that histamine is most likely one of the mediators of anaphylaxis in this model.

Mast Cell Degranulation in C3H Mice:

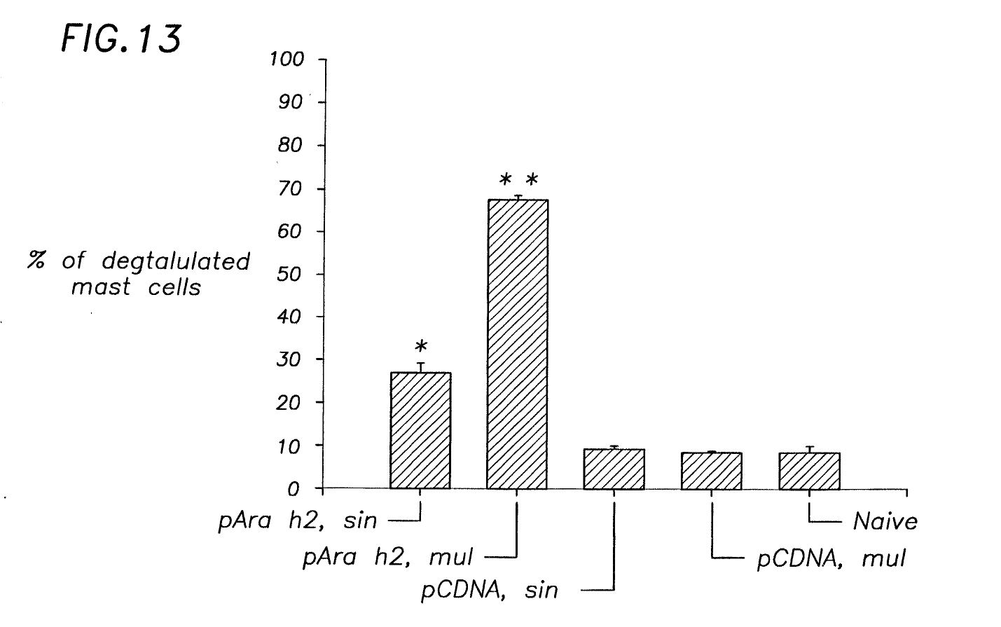

Histologic analysis of mouse ear tissue showed a significant increase in the number of degranulated mast cells in pAra h 2-immunized mice following PN injection when compared with control mice (data not shown). Consistent with the findings of elevated levels of plasma histamine, the percentage of degranulated mast cells in mice given multiple pAra h 2 immunizations was markedly greater than in singly immunized mice (FIG. 13). These data demonstrate that mast cell degranulation and consequent histamine release are involved in the induction of anaphylaxis in pAra h 2-immunized C3H mice following PN injection.

Pca Reactions in C3H Mice:

The virtual absence of IgE and the high levels of IgG1 induced by pAra h 2 immunization, together with the association between the level of IgG1 and the severity of anaphylactic reactions (FIGS. 10 and 11) suggested that peanut-induced anaphylactic shock in the C3H mouse model is IgG1 mediated. To further evaluate this hypothesis, PCA testing was performed as described in Materials and Methods. PCA reactions were induced by heat-inactivated and non-heated sera from pAra h 2-immunized C3H mice (Table IV). In contrast, no PCA reactions were found in peanut-injected mice that received mock pDNA immune sera or PBS. These results demonstrate that IgG1, but not IgE was the reagenic Ab in this model.

TABLE IV
PCA reactions in C3H mice*

| Donor | Heat | Diameter (cm) | Positive Reaction | |
|---|---|---|---|---|
| Immunizations | Inactivation | (mean = SE) | n/total | % |
| pAra h 2 | + | 2.67 ± 0.21 | 6/6 | 100 |
| pAra h 2 | − | 2.75 ± 0.17 | 6/6 | 100 |
| pcDNA | − | 0.14 ± 0.06 | 0/5 | 0 |
| PBS | − | 0.12 ± 0.05 | 0/5 | 0 |

*Naive C3H mice in each group (n = 5-6) as indicated received heated or non-heated pAra h 2 immune sera. mock DNA (pcDNA) immune sera. or PBS followed by PN/Evan's blue dye administration. PCA reactions were scored as described in Materials and Methods.

Isotype Profile of Ag-Specific Abs Induced By Pomc Immunization of C3H Mice:

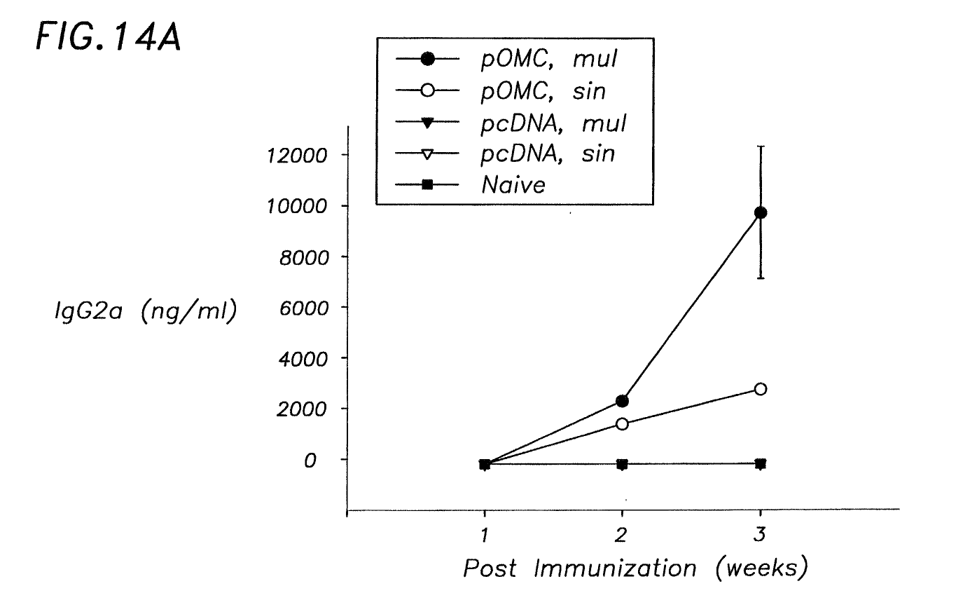

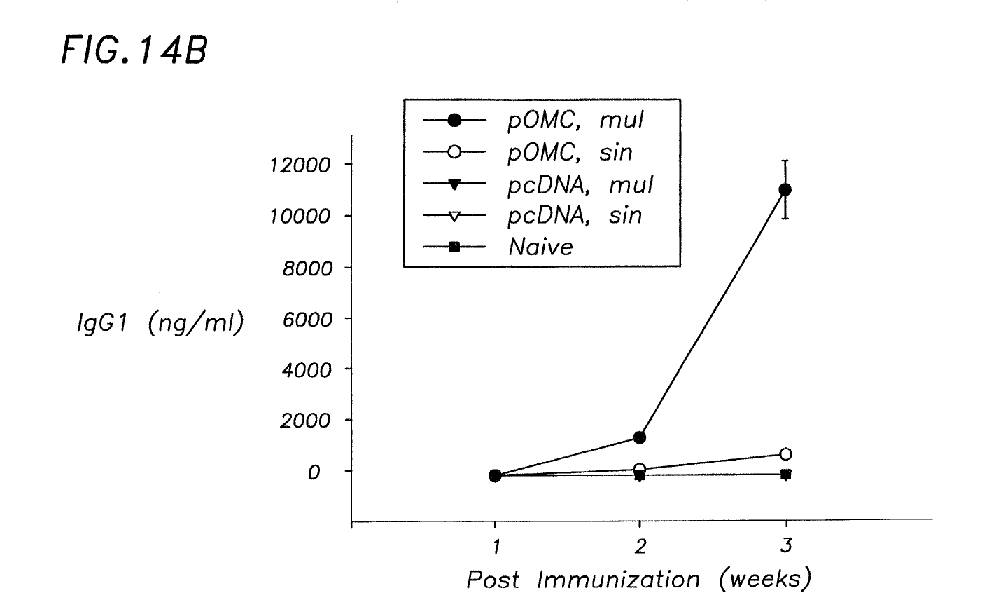

To determine whether the induction of Ara h 2-specific IgG1 in pAra h 2-immunized C3H mice is specific to peanut allergen, C3H mice were multiply immunized with pOMC, the plasmid DNA encoding the major egg allergen protein, ovomucoid. The Ab responses were measured kinetically after immunization. Similar to pAra h 2-immunized C3H mice, both IgG1 and IgG2a Ab levels were markedly increased 2 wk after immunization (FIG. 14). At 3 wk, the level of ovomucoid-specific IgG1 levels in the multiply immunized group was about 32-fold greater than that in the singly immunized group, whereas IgG2a levels in multiply immunized mice were threefold greater than in singly immunized mice. Challenge of pOMC-immunized mice with ovomucoid also resulted in severe anaphylactic reactions (data not shown). These results demonstrate that pDNA immunization-induced IgG1 Ab responses in C3H mice are not unique to pAra h 2.

Strain-Dependent Reactions to Peanut Protein Injection Following Para H 2 DNA Immunization:

Since the results described above differed from those of the two previous studies of allergen gene immunization, in which different rodent models were used (Hsu et al. Nat. Med. 2:540, 1996; Raz et al. Proc. Natl. Acad. Sci. USA 93:5141, 1996), we hypothesized that the consequences of allergen gene immunization may be strained dependent. To evaluate this possibility, we employed AKR and BALB/c mice, utilizing the same multiple pDNA immunization protocol used in C3H mice. In contrast to C3H mice, peanut protein injection of AKR or BALB/c mice at 3 or 5 wk following pAra h 2 DNA immunization did not elicit any sign of anaphylaxis (Table V).

TABLE V
Anaphylactic reactions to PN injection in different strains of mice
following pAra h 2 DNA immunization*

| | |
|---|---|
| 3 wk | 5 wk |

| Strain | n/total | % | n/total | % |
|---|---|---|---|---|
| C3H | 10/10 | 100 | 5/5 | 100 |
| AKR | 0/10 | 0 | 0/8 | 0 |
| BALB/c | 0/10 | 0 | 0/6 | 0 |

*Mice (n = 5-10) in each group as indicated received i.p. injection of PN at 3 or 5 wk following pDNA multiple immunization. The incidence of anaphylaxis in each group of mice was calculated and described as morbidity rate.

Kinetics and Isotype Profile of Ag-Specific Abs Induced By Pdna Immunization of Akr, Balb/c, and C3H Mice:

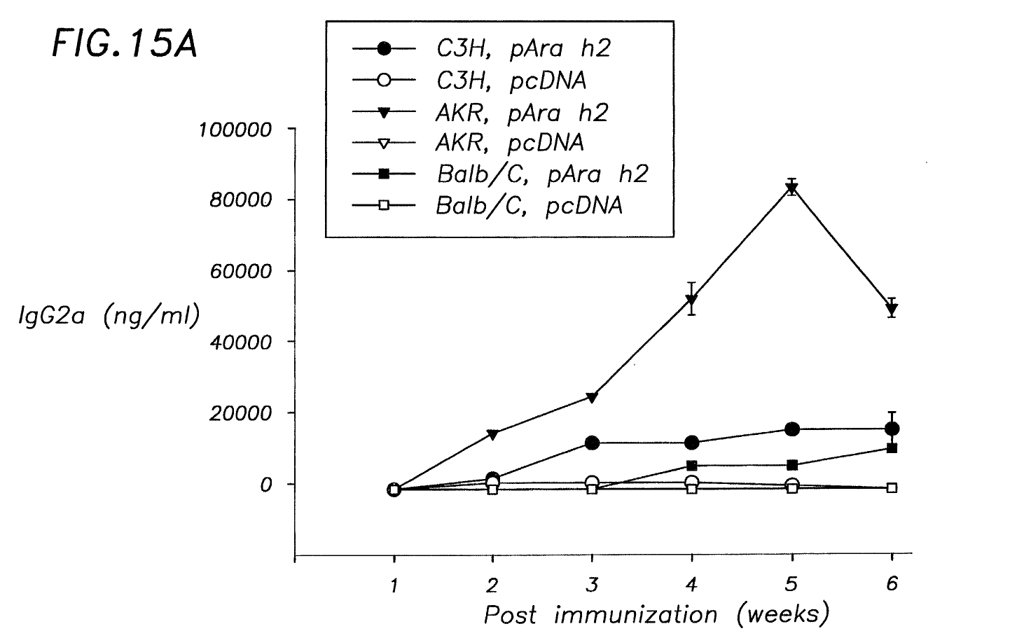

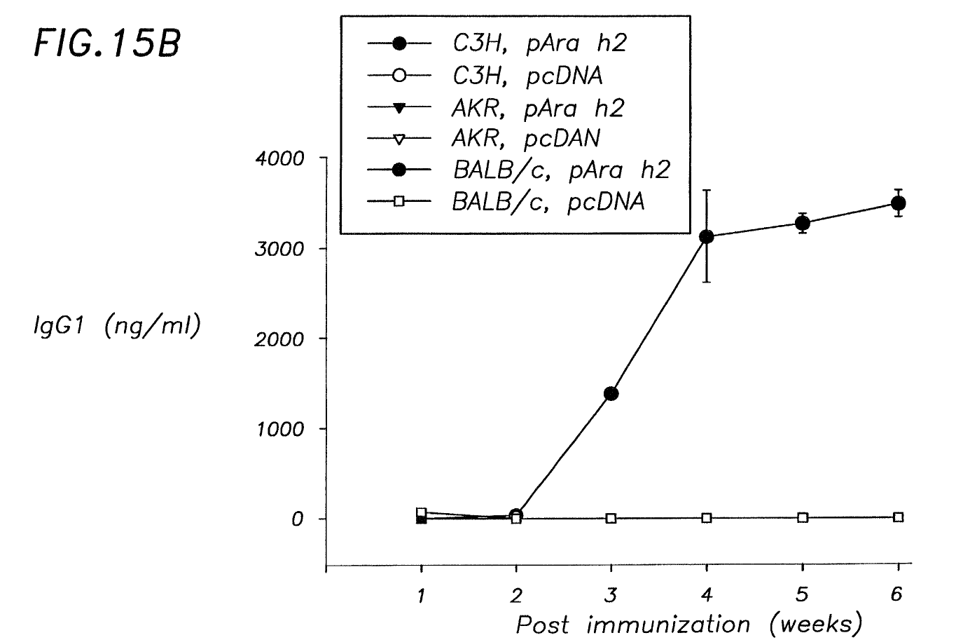

To elucidate the immunologic mechanisms underlying these different types of responses to AKR, BALB/c and C3H mice, we examined the kinetics of the Ara h 2-specific IgG2a, IgG1 and IgE Abs from week 1 through week 6 following multiple doses of pDNA immunization (FIG. 15). In AKR mice, IgG2a was markedly increased at 2 wk and reached a peak at 5 wk. In BALB/c mice, no IgG2a Ab was present until week 4; the peak level was found at week 6. No IgG1 or IgE Ara h 2-specific Abs were detected following pAra h 2 immunization at any time point in either AKR or BALB/c mice. Although BALB/c mice presented a similar pattern of IgG2a responses as AKR mice, the responses occurred slightly later and were weaker. In contrast to the IgG isotype profile in AKR and BALB/c mice, both IgG2 and IgG1 were increased significantly in C3H mice at week 3, and peaked at week 3 for IgG2a and at week 4 for IgG1. No significant decreased in the level of either IgG2a or IgG1 was observed thereafter. Furthermore, the levels of IgG2a in C3H mice were significantly lower than that in AKR mice. These findings demonstrate that the variability of Ab responses to pDNA immunization is primarily strain dependent.

Cell Cytokine Profiles Induced By Para H 2 Immunization:

To determine whether the different Ab responses of these three strains were related to differential production of T cell cytokines, cytokines produced by spleen cells were measured 3 wk following multiple pAra h 2 immunization. Since cytokine production in culture following PN stimulation revealed that levels of IFN-γ peaked at 72 h, IL-4 increased significantly at 24 h, but did not decrease significantly thereafter, and IL-5 was not detected at any time point, Table VI depicts supernatants cytokine levels after 72 h of culture. Levels of IFN-γ were markedly increased in Con A-stimulated cultures from all three strains. Levels of IFN-γ in PN-stimulated cultures, were also significantly higher than unstimulated cultures from all three strains (p<0.001 in C3H; 0.01 in AKR; 0.05 in BALB/c). C3H spleen cells produced approximately twice

TABLE VI
Cytokine secretion spleen cells from different stains of mice following
pAra h 2 immunization*

| | IFN-γ (pg/ml) | | | IL-4 (/ml) | | | IL-5 (pg/ml) | | |
|---|---|---|---|---|---|---|---|---|---|
| Strain | PN | Con A | Control | PN | Con A | Control | PN | Con A | Control |
| C3H | 749 ± 101 | >6000 | >7.8 | 115 ± 5 | 389 ± 21 | 56 ± 1 | <7.8 | 564 ± 10 | <7.8 |
| AKR | 397 ± 69 | >6000 | 133 ± 15 | 105 ± 7 | 207 ± 2 | 70 ± 4 | <7.8 | 131 ± 14 | <7.8 |
| BALB/c | 360 ± 57 | >6000 | 296 ± 4 | 68 ± 2 | 712 ± 51 | 52 ± 3 | <7.8 | 385 ± 12 | <7.8 |

*Spleen cells from the mice (n = 2-3) at 3 wk after pAra h 2 multiple immunization were cultured in the presence or absence of Con A or PN Levels of IFN-γ, IL-4 and IL-5 in 72-h culture supernatants were measured by ELISA and calculated by comparison with a standard curve. The lower and upper levels of the assay in this experiment were 7.8 and 6000 pg/ml.

as much PN-induced IFN-γ as AKR and BALB/c cells. Although Con A stimulation significantly increased (p <0.01) IL-4 secretion in cultures from all three strains. PN stimulation resulted in similar significantly increased (p<0.02) levels of IL-4 production by the cells from C3H and AKR, but not from BALB/c. Levels of IL-5 were increased significantly in Con A-stimulated cultures (p<0.01) from all three strains. However, IL-5 was not detectable in PN-stimulated cultures from any of the three strains. These results indicate that Th1/Th2 cytokine production in splenic cells does not reflect the differential expression of pAra h 2-induced IgG1 or IgG2a among the three strains in these experiments.

### Example 4

### Mapping IgE Binding Sites in Peanut Antigens

Introduction

This Example describes the definition and analysis of IgE binding sites within peanut protein Ala antigens. The information presented may be utilized in accordance with the present invention, for example, to prepare one or more antigen fragments, or collections thereof, lacking one or more peanut antigen IgE binding site. In general, any of a variety of methods (e.g., immunoprecipitation, immunoblotting, cross-linking, etc.) can be used to map IgE binding sites in antigens (see, for example, methods described in Coligan et al. (eds.) Current Protocols in Immunology, Wiley & Sons, and references cited therein, incorporated herein by reference). Generally, an antigen or antigen fragment (prepared by any available means such as, for example, chemical synthesis, chemical or enzymatic cleavage, etc.) is contacted with serum from one or more individuals known to have mounted an immune response against the antigen. Where the goal is to map all observed IgE binding sites, it is desirable to contact the antigen or antigen fragment, simultaneously or serially, with sera from several different individuals since different epitopes may be recognized in different individuals. Also, different organisms may react differently to the same antigen or antigen fragments; in certain circumstances it may be desirable to map the different IgE binding sites observed in different organisms.

It will be appreciated that an IgE binding site that is strongly recognized in the context of an intact antigen may not be strongly bound in an antigen fragment even though that fragment includes the region of the antigen corresponding to the binding site. As will be clear from context, in some circumstances an antigen fragment is considered to contain an IgE binding site whenever it includes the region corresponding to an IgE binding site in the intact antigen; in other circumstances, an antigen fragment is only considered to have such a binding site if physical interaction has actually been demonstrated as described herein.

From studies mapping both murine and human IgE binding sites within peanut protein antgens, the human and murine sites overlap to a great extent demonstrating the usfulness of the mouse as a model of anaphylaxis.

Materials and Methods

Ige Immunoblot Analysis:

Membranes to be blotted were prepared either by SDS-PAGE (performed by the method of Laemmli Nature 227:680-685, 1970) of digested peanut antigen or by synthesis of antigen peptides on a derivativized cellulose membrane). SDS-PAGE gels were composed of 10% acrylamide resolving gel and 4% acrylamide stacking gel. Electrophoretic transfer and immunoblotting on nitrocellulose paper was performed by the procedures of Towbin (Proc. Natl. Acad. Sci. USA 76:4350-4354, 1979).

For mapping of human IgE binding sites, the blots were incubated with antibodies (serum IgE) from 15-18 patients with documented peanut hypersensitivity. Each of the individuals had a positive immediate skin prick test to peanut and either a positive, double-blind, placebo-controlled food challenge or a convincing history of peanut anaphylaxis (laryngeal edema, severe wheezing, and/or hypotension). At least 5 ml of venous blood was drawn from each patient and allowed to clot, and the serum was collected. All studies were approved by the Human Use Advisory Committee at the University of Arkansas for Medical Sciences. Serum was diluted in a solution containing TBS and 1% bovine serum albumin for at least 12H at 4° C. or for 2 h at room temperature. The primary antibody was detected with ¹²⁵I-labeled anti-IgE antibody (Sanofi Diagnostics Pasteur Inc., Paris, France).

For mapping of murine IgE binding sites, a blot containing overlapping 13mer peptides, offset by 2 amino acids, was incubated with serum from mice described in Example 2.

Peptide Synthesis:

Individual peptides were synthesized on a derivativized cellulose membrane using Fmoc amino acid active esters according to the manufacturer's instructions (Genosys Biotechnologies, Woodlands, Tex.). Fmoc-amino acid derivatives were dissolved in 1-methyl-2-pyrrolidone and loaded on marked spots on the membrane. Coupling reactions were followed by acetylation with a solution of 4% (v/v) acetic anhydride in N,N-dimethyl form amide (DMF). After acetylation, Fmoc groups were removed by incubation of the membrane in 20% (v/v) piperdine in DMF. The membrane was then stained with bromophenol blue to identify the location of the free amino groups. Cycles of coupling, blocking, and deprotection were repeated until the peptides of the desired length were synthesized. After addition of the last amino acid in the peptide, the amino acid side chains were deprotected using a solution of dichloromethane/trifluoroacetic acid/triisobutylsilante (1110/0.05). Membranes were either probed immediately or stored at −20° C. until needed.

Results

Human IgE binding sites have previously been mapped for Ara h 1 (Burks et al., J. Clin. Invest. 96:1715-1721, 1995; U.S. Ser. No. 90/141,220, filed Aug. 27, 1998, each of which is incorporated herein by reference) and Ara h 2 (Stanley et al., Arch. Biochem. Biophys. 342:244-253, 1997; U.S. Ser. No. 90/141,220, filed Aug. 27, 1998, each of which is incorporated herein by reference). We have also mapped such epitopes for Ara h 3 (Rabjohn et al., J. Clin. Invest. 103:535-542, 1999; U.S. Ser. No. 90/141,220, filed Aug. 27, 1998, each of which is incorporated herein by reference). As described in Example 2, we have also mapped murine IgE binding sites for Ara h 2, by probing filters containing overlapping 20mers, offset by 5 amino acids, that span the Ara h 2 sequence with serum from mice sensitized to recombinant Ara h 2.

The results of these studies are summarized below in Tables (essential residues are underlined).

TABLE VII
IgE Binding Epitopes in Ara h 1

| EPITOPE NUMBER | SEQUENCE | POSITION |
|---|---|---|
| 1 | AKSSPYQKKT | 25-34 |
| 2 | QEPDDLKQKA | 48-57 |
| 3 | LEYDPRLVYD | 65-74 |
| 4 | GERTRGRQPG | 89-98 |
| 5 | PGDYDDDRRQ | 97-106 |
| 6 | PRREEGGRWG | 107-116 |
| 7 | REREEDWRQP | 123-132 |
| 8 | EDWRRPSHQQ | 134-143 |
| 9 | QPRKIRPEGR | 143-152 |
| 10 | TPGQFEDFFP | 294-303 |
| 11 | SYLQEFSRNT | 311-320 |
| 12 | FNAEFNEIRR | 325-334 |
| 13 | EQEERGQRRW | 344-353 |
| 14 | DITNIPINLRE | 393-402 |
| 15 | NNFGKLFEVK | 409-418 |
| 16 | GTGNLELVAV | 461-470 |
| 17 | RRYTARLKEG | 498-507 |
| 18 | ELHLLGFGIN | 525-534 |
| 19 | HRIFLAGDKD | 539-548 |
| 20 | IDQIEKQAKD | 551-560 |
| 21 | KDLAFPGSGE | 559-568 |
| 22 | KESHFVSARP | 578-587 |
| 23 | PEKESPEKED | 597-606 |

TABLE VIII
IgE Binding Epitopes in Ara h 2

| SEQUENCE OF HUMAN EPITOPE | SEQUENCE OF MOUSE EPITOPE | |
|---|---|---|
| (NUMBER) | (NUMBER) | POSITION |
| HASARQQWEL (1) | LFLLAAH (1) | H15-24 |
| | | M9-15 |
| QWELQGDRRC (2) | RQQWELQGDRR (2) | H21-28 |
| | | M19-29 |
| DRRCQSQLER (3) | RCQSQLERA (3) | H27-36 |
| | | M29-37 |
| LRPCEQHLMQ (4) | | H39-48 |
| KIQRDEDSYE (5) | DEDSYERDP (4) | H49-56 |
| | | M53-61 |
| YERDPYSPSQ (6) | YERDPYSPS (5) | H59-64 |
| | | M57-65 |
| SQDPYSPSPY (7) | YSPSPYD (6) | H65-72 |
| | | M69-75 |
| DRLQGRQQEQ (8) | QQEQQFK (7) | H117-122 |
| | | M121-127 |
| KRELRNLPQQ (9) | KRELRNLPQ (8) | H127-132 |
| | | MM127-135 |
| | RNLPQQCGL (9) | M131-139 |
| | CGLRAPQ (10) | M137-143 |
| QRCDLDVESG (10) | QRCDLDV (11) | H143-152 |
| | | M143-149 |

Human Ara h 2 epitopes (6) and (7), and mouse Ara h 2 epitopes (5) and (6) were considered to be immunodominant because, in each case, the two epitopes combined contributed about 40-50% of the observed IgE reactivity (as determined by densitometric analysis of the blot). Human epitope (3) was also considered to be immunodominant, as it contributed as much as about 15% of the IgE reactivity. No other mouse or human epitope contributed more than about 10% of the reactivity.

TABLE IX
IgE Binding Epitopes in Ara h 3

| EPITOPE NUMBER (FRACTION | | |
|---|---|---|
| OF PATIENTS WITH IGE THAT | | |
| BIND) | SEQUENCE | POSITION |
| 1 (25%) | IETWNPNNQEFECAG | 33-47 |
| 2 (38%) | GNIFSGFTPEFLEQA | 240-254 |
| 3 (100%) | VTVRGGLRILSPDRK | 279-293 |
| 4 (38%) | DEDEYEYDEEDRRRG | 303-317 |

Epitome 3 of Ara h 3 was designated as immunodominant because it was recognized by IgE in sera from all 10 patients tested.

### Example 5

### Collections of Ara h 2 Peptides

5/20 Native

A collection of 28 peptides, each 20 amino acids long and staggered by 5 amino acids, spanning the sequence of the native Ara h 2 protein was prepared as described above. Table X present the sequences of the individual peptides:

TABLE X
5/20 Native Ara h 2 Peptides

| PEPTIDE NO | SEQ ID NO: | SEQUENCE |
|---|---|---|
| 1 | | LTILVALALFLLAAHASARQ |
| 2 | | ALALFLLAAHASARQQWELQ |
| 3 | | LLAAHASARQQWELQGDRRC |
| 4 | | ASARQQWELQGDRRCQSQLE |
| 5 | | QWELQGDRRCQSQLERANLR |
| 6 | | GDRRCQSQLERANLRPCEQH |
| 7 | | QSQLERANLRPCEQHLMQKI |
| 8 | | RANLRPCEQHLMQKIQRDED |
| 9 | | PCEQHLMQKIQRDEDSYERD |
| 10 | | LMQKIQRDEDSYERDPYSPS |
| 11 | | QRDEDSYERDPYSPSQDPYS |
| 12 | | SYERDPYSPSQDPYSPSPYD |
| 13 | | PYSPSQDPYSPSPYDRRGAG |
| 14 | | QDPYSPSPYDRRGAGSSQHQ |
| 15 | | PSPYDRRGAGSSQHQERCCN |
| 16 | | RRGAGSSQHQERCCNELNEF |
| 17 | | SSQHQERCCNELNEFENNQR |
| 18 | | ERCCNELNEFENNQRCMCEA |
| 19 | | ELNEFENNQRCMCEALQQIM |
| 20 | | ENNQRCMCEALQQIMENQSD |
| 21 | | CMCEALQQIMENQSDRLQGR |
| 22 | | LQQIMENQSDRLQGRQQEQQ |
| 23 | | ENQSDRLQGRQQEQQFKREL |
| 24 | | RLQGRQQEQQFKRELRNLPQ |
| 25 | | QQEQQFKRELRNLPQQCGLR |
| 26 | | FKRELRNLPQQCGLRAPQRC |
| 27 | | RNLPQQCGLRAPQRCDLDVE |
| 28 | | QCGLRAPQRCDLDVESGGRD |

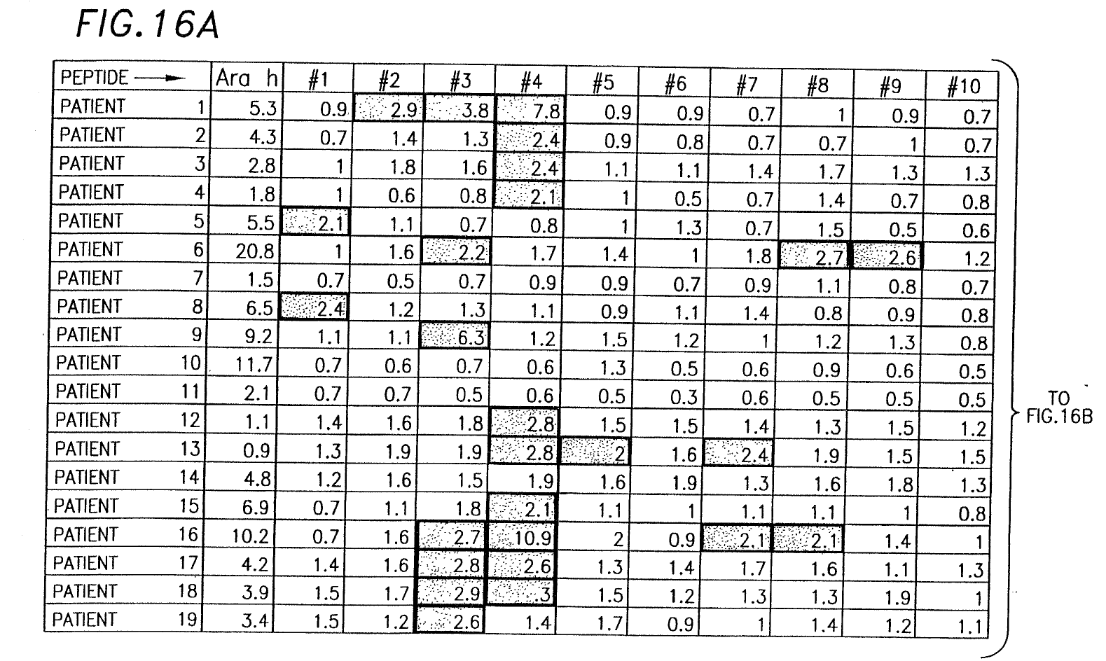

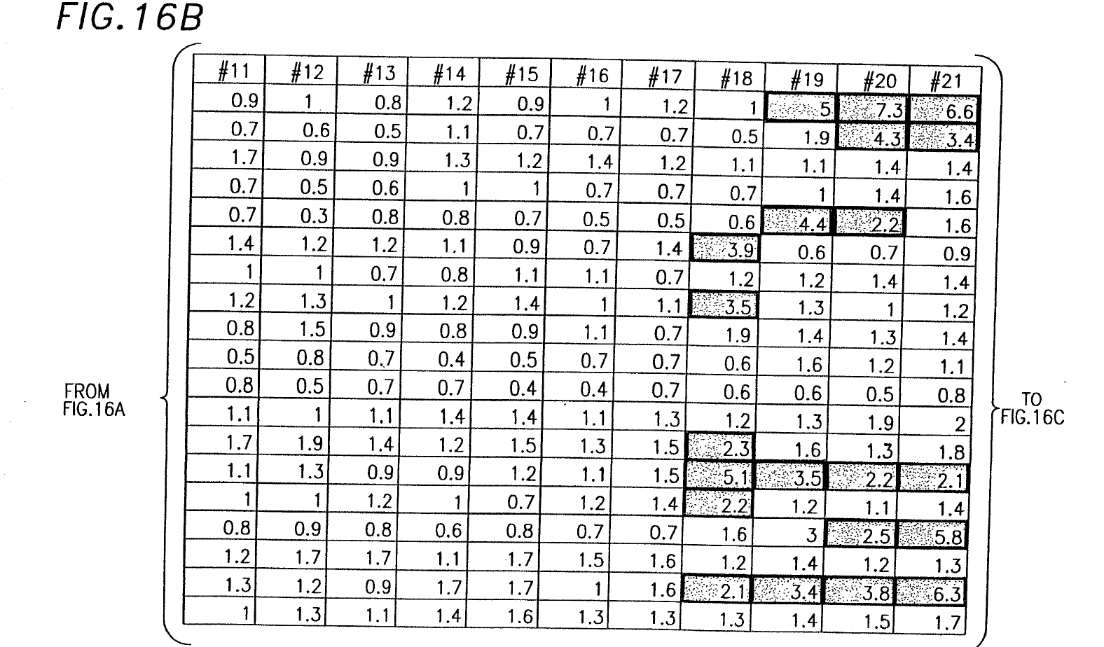

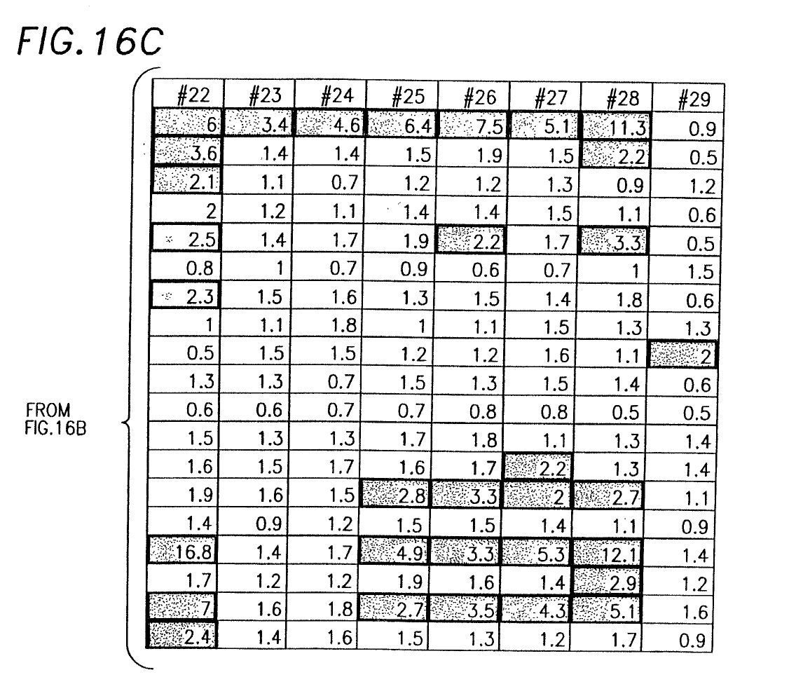

Each of these peptides was tested for its ability to stimulate human T cells. The results are shown in FIG. 16. Each peptide was tested, using standard different techniques, on 19 different T cell preparations. Positive scores, defined as a T cell stimulation index of >2, are indicated by shading. As can be seen, peptides 1-9 (especially 3 and 4) and 18029 (especially 18-22 and 25-28) have significant T cell stimulation capability; peptides, 10-17 do not show such activity.

5/15 Modified

A collection of 24 peptides, each (except for the last) 15 amino acids long and staggered by 5 amino acids, spanning the sequence of a modified Ara h 2 protein, in which all IgE binding sites were disrupted by alanine substitution can be synthesized. Table XI presents the sequences of the individual peptides; modified residues are indicated by underlining.

TABLE XI
5/15 Modified Ara h 2 Peptides

| | | |
|---|---|---|
| PEPTIDE NO | SEQ ID NO: | SEQUENCE |

| | |
|---|---|
| 1 | LTILVALALFLLAAH |
| 2 | ALALFLLAAHASARQ |
| 3 | LLAAHASARQQAELQ |
| 4 | ASARQQAELQGDRRC |
| 5 | QQAELQGDRRCQSQLA |
| 6 | QGDRRCQSQLARANLR |
| 7 | QSQLARANLRACEAH |
| 8 | RANLRACEAHLMQKI |
| 9 | ACEAHLMQKIQADED |
| 10 | LMQKIQADEDSYERA |
| 11 | QADEDSYERAPYSPS |
| 12 | SYERAPYSPSQAPYS |
| 13 | PYSPSQAPYSPSPYD |
| 14 | QAPYSPSPYDRRGAG |
| 15 | PSPYDRRGAGSSQHQ |
| 16 | RRGAGSSQHQERCCN |
| 17 | SSQHQERCCNQQEQQ |
| 18 | ERCCNQQEQQFKREA |
| 19 | QQEQQFKREARNLPQ |
| 20 | FKREARNLPQQCGLR |
| 21 | RNLPQQCGLRAPQRC |
| 22 | QCGLRAPQRCDADVE |
| 23 | APQRCDADVESGGRD |
| 24 | DADVESGGRDRY |

5/20 Native, Depleted for ≧2 Human Sites

One strategy for reducing the effective IgE binding activity of a collection of overlapping Ara h 2 peptides is to remove from the collection those peptide that include two or more IgE binding sites, and therefore have the ability to cross-link anti-Ara h 2 IgE molecules. Individual peptides could be tested for their ability to simultaneously bind to two or more IgE molecules could be identified by direct testing of IgE binding and/or cross-linking (e.g., histamine release). However, in the present Example, we simply designate those peptides that contain two complete IgE binding sites as determined by sequence analysis alone, relying on the above-described analyses to define the IgE binding sites. Under this analysis, peptides 3, 5, and 12 from Table X should be removed from the collection.

5/20 Native, Depleted for Immunodominant Epitopes

As mentioned above, human epitopes (6) and (7) (or mouse epitopes (5) and (6)) together are responsible for more than 40-50% of the IgE binding activity observed when human sera are tested against a panel of overlapping Ara h 2 peptides (see Stanley et al., Arch. Biochem. Biophys. 342:244-253, 1997, incorporated herein by reference). In certain embodiments of the invention, all peptides containing part or all of these sequences are removed from the 5/20 collection discussed above, to produce a 5/20 collection depleted of major immunodominant epitopes. That is, peptides 11-14, corresponding to amino acids 51-85, are removed from the collection. Interestingly, these peptides are not particularly active at stimulating T cell proliferation.

5/20 Native, Depleted for any Intact Human Sites

In yet another embodiment of the invention, the above-described 5/20 collection of native Ara h 2 peptides is depleted for those peptides that contain an intact IgE binding site as defined above in Example 4. Such depletion removes peptides 2-13 and 22-28 from the collection.

### Example 6

### Desensitization of PN-Sensitized Mice Using Ara h 2 Peptides Introduction

This Example describes the use of a collection of antigen fragments (of the Ara h 2 protein) to desensitize individuals to preanut allergy. The Example also shows desensitization using a modified Ara h 2 protein whose IgE binding sites have been disrupted. The results with modified protein antigen are readily generalizable to peptide fragments, as described herein.

Materials and Methods

Mice and Reagents:

Female C3H/HeJ mice, 5-6 weeks of age were purchased from the Jackson Laboratory (Bar Harbor, Me.) and maintained on PN-free chow, under specific pathogen-free conditions. Standard guidelines for the care and use of animals was followed.

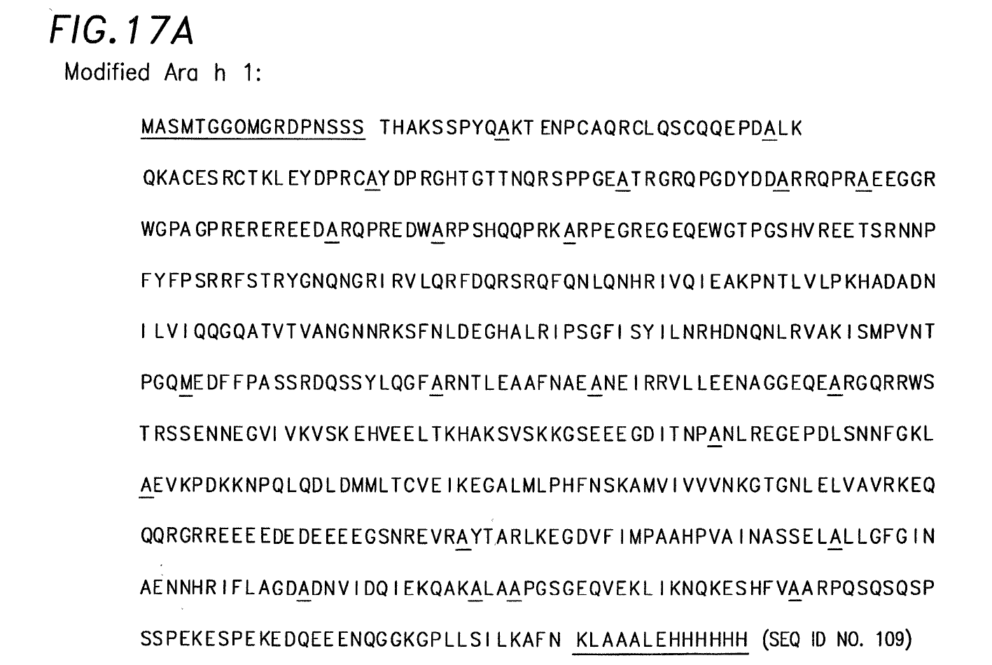

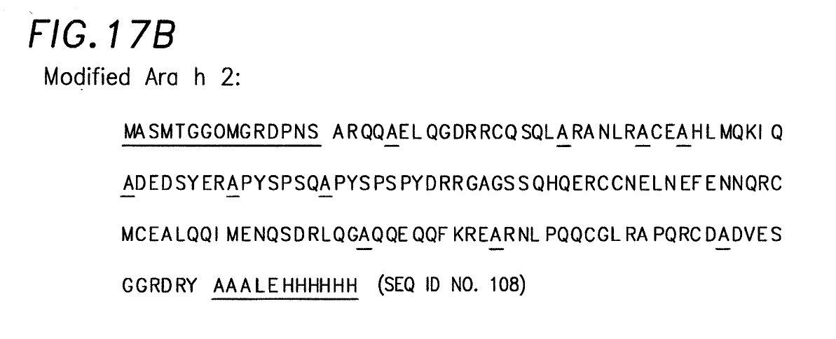

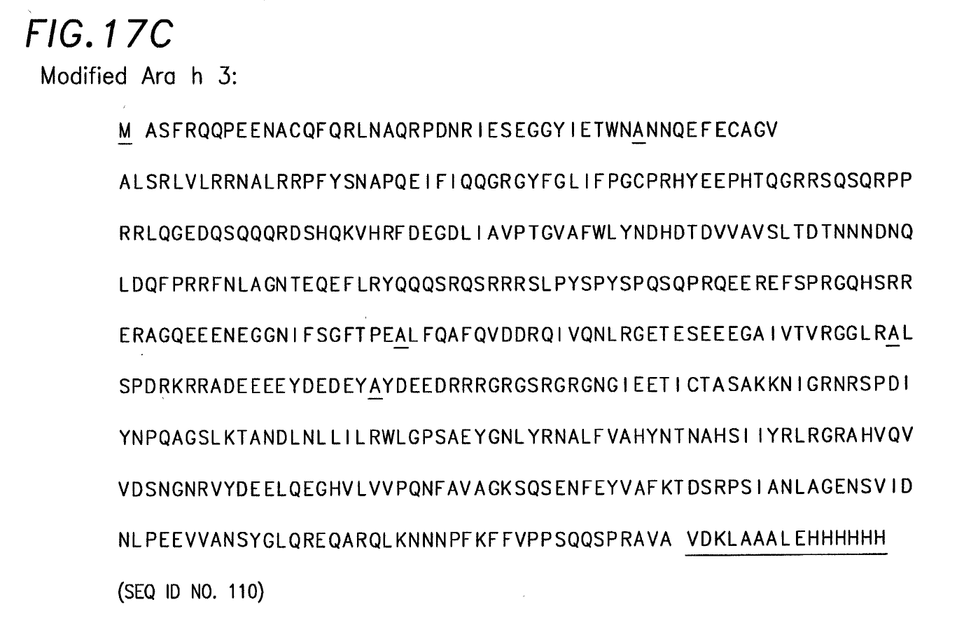

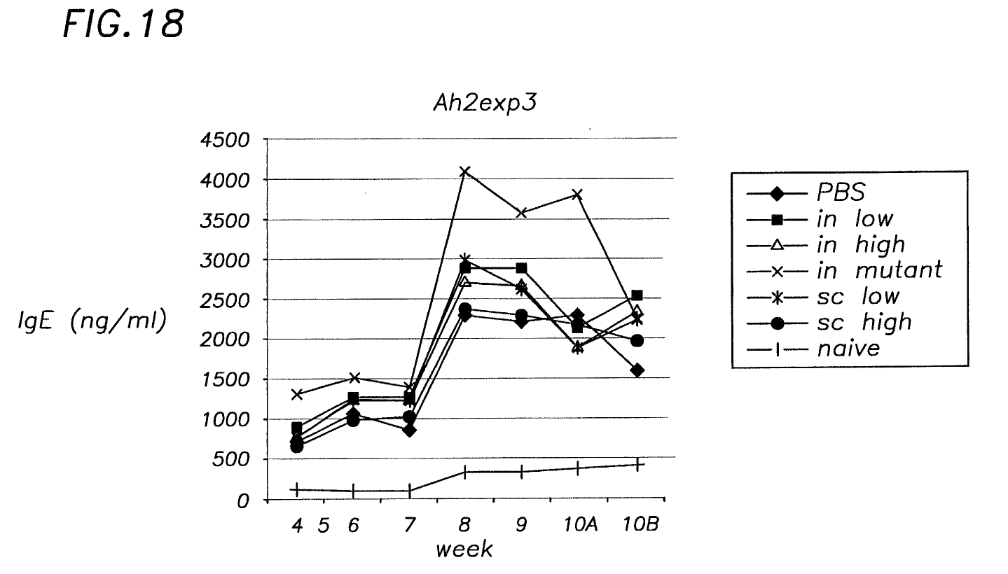

Ara h 2 protein was purified as described by Burks et al. (J. Allergy Clin. Immunol. 8: 172-179, 1992, incorporated herein by reference). Modified Ara h 2 was prepared as described in U.S. Ser. No. 09/141,220 filed Aug. 27, 1998, incorporated herein by reference. The sequence of the modified Ara h 2 differed from that of natural Ara h 2 as indicated in FIG. 17 (altered positions are underlined). The Ara h 2 peptide collection was the 5/20 collection described above in Example 4.

Sensitization:

Mice were sensitized by ig feeding with 5 mg of Ara h 2 plus 0.3 μg/gm body weight of cholera toxin (CT) as an adjuvant and were boosted twice, at weeks 1 and 3. Intragastric feeding was performed by means of a stainless steel blunt feeding needle as described by Li et al., J. Allergy Clin. Immunol. 103:206, 1999, incorporated herein by reference). Control mice received either CT alone or sham treatment.

Desensitization:

Two weeks after sensitization, mice were treated with intranasal or subcutaneous peptide mix (either 2 μg or 20 μg), or with intranasal modified Ara h 2 (2 μg). One set of control mice was treated with intranasal wild type Ara h 2; another set was mock treated.

Challenge:

Two weeks later. desensitized mice were challenged orally with 5 mg of wild type Ara h 2, divided into two doses of 2.5 mg 30 min apart.

Assays:

Hypersensitivity testing and IgE measurement were performed as described above in Example 2. Plasma histamine levels and airway responsiveness were also assayed, as were Ara h 2-specific IgE and IgG2 levels.

Rechallenge:

The mice that were sensitized, desensitized, and challenged as described above in Example were rechallenged with Ara h 2 protein 3 weeks later.

Results

As shown in Figure XX, anti-Ara h 2 IgE levels in mice exposed to native Ara h 2 rose four fold during the “desensitization period”. By contrast, these IgE levels did not increase significantly in mice exposed to low or high dose peptides, and actually decreased almost two-fold in mice exposed to modified Ara h 2. Moreover, significant protection from anaphylaxis was observed with both the high dose peptides and the modified protein. In order to determine whether this protection were long term, we rechallenged the mice several (three) weeks later. As shown below in Table XII, the observed protection was long term:

TABLE XII

| | ANTI-ARA H 1 IGE LEVELS | SEVERITY OF ANAPHYLACTIC |
|---|---|---|
| | DURING 3 WEEKS BETWEEN | SYMPTOMS AS COMPARED |
| “VACCINE” | CHALLENGES | WITH FIRST CHALLENGE |
| sham | increased | worse |
| low [native peptides] | increased | worse |
| 20mers, 5aa stagger | | |
| 2 μg/mouse | | |
| high [native peptides] | no increase | no change |
| 20mers, 5aa stagger | | |
| 20 μg/mouse | | |
| modified protein | modest increase | no significant change |

These results clearly demonstrate that a collection of Ara h 2 peptides containing substantially all of the structural features of Ara h 2, can desensitize individuals allergic to Ara h 2. A modified Ara h 2 protein can have similar effect, indicating that peptide collections lacking one or more effective IgE binding sites should also be useful desenitization tools.

### Other Embodiments

Those of ordinary skill in the art will readily appreciate that the foregoing represents merely certain preferred embodiments of the invention. Various changes and modifications to the procedures and compositions described above can be made without departing from the spirit or scope of the present invention, as set forth in the following claims.

[image]

[image]

[image]

[image]

[image]

[image]

[image]

[image]

[image]

[image]

[image]

[image]

[image]

[image]

[image]

[image]

[image]

[image]

[image]

1. Marsh, D. G., and L. R. Freidhoff. 1992. ALBE, an allergen database. IUIS, Baltimore, Md., Edition 1.0.

2. Marsh, D. G., L. Goodfriend, T. P. King, H. Lowenstein, and T. A. E. Platts-Mills. 1986. Allergen nomenclature. Bull WHO 64:767-770.

3. King, T. P., P. S. Norman, and J. T. Cornell. 1964. Isolation and characterization of allergen from ragweed pollen. II. Biochemistry 3:458-468.

4. Lowenstein, H. 1980. Timothy pollen allergens. Allergy 35:188-191.

5. Aukrust, L. 1980. Purification of allergens in Cladosporium herbarum. Allergy 35:206-207.

6. Demerec, M., E. A. Adelberg, A. J. Clark, and P. E. Hartman. 1966. A proposal for a uniform nomenclature in bacterial genetics. Genetics 54:61-75.

7. Bodmer, J. G., E. D. Albert, W. F. Bodmer, B. Dupont, H. A. Erlich, B. Mach, S. G. E. Marsh, W. R. Mayr, P. Parham, T. Sasuki, G. M. Th. Schreuder, J. L. Strominger, A. Svejgaard, and P. I. Terasaki. 1991. Nomenclature for factors of the HLA system, 1990. Immunogenetics 33:301-309.

8. Griffith, I. J., J. Pollock, D. G. Klapper, B. L. Rogers, and A. K. Nault. 1991. Sequence polymorphism of Amb a I and Amb a II, the major allergens in Ambrosia artemisiifolia (short ragweed). Int. Arch. Allergy Appl. Immunol. 96:296-304.

9. Roebber, M., D. G. Klapper, L. Goodfriend, W. B. Bias, S. H. Hsu, and D. G. Marsh. 1985. Immunochemical and genetic studies of Amb t V (Ra5G), an Ra5 homologue from giant ragweed pollen. J. Immunol. 134:3062-3069.

10. Metzler, W. J., K. Valentine, M. Roebber, M. Friedrichs, D. G. Marsh, and L. Mueller. 1992. Solution structures of ragweed allergen Amb t V. Biochemistry 31:5117-5127.

11. Metzler, W. J., K. Valentine, M. Roebber, D. G. Marsh, and L. Mueller. 1992. Proton resonance assignments and three-dimensional solution structure of the ragweed allergen Amb a V by nuclear magnetic resonance spectroscopy. Biochemistry 31:8697-8705.

12. Goodfriend, L., A. M. Choudhury, J. Del Carpio, and T. P. King. 1979. Cytochromes C: New ragweed pollen allergens. Fed. Proc. 38:1415.

13. Ekramoddoullah, A. K. M., F. T. Kisil, and A. H. Sehon. 1982. Allergenic cross reactivity of cytochrome c from Kentucky bluegrass and perennial ryegrass pollens. Mol. Immunol. 19:1527-1534.

14. Ansari, A. A., E. A. Killoran. and D. G. Marsh. 1987. An investigation of human response to perennial ryegrass (Lolium perenne) pollen cytochrome c (Lol p X). J. Allergy Clin. Immunol. 80:229-235.

15. Morgenstern, J. P., I. J. Griffith, A. W. Brauer, B. L. Rogers, J. F. Bond, M. D. Chapman, and M. Kuo. 1991. Amino acid sequence of Fel d I, the major allergen of the domestic cat: protein sequence analysis and cDNA cloning. Proc. Natl. Acad. Sci. USA 88:9690-9694.

16. Griffith, I. J., S. Craig, J. Pollock, X. Yu, J. P. Morgenstern, and B. L.Rogers. 1992. Expression and genomic structure of the genes encoding FdI, the major allergen from the domestic cat. Gene 113:263-268.

17. Weber, A., L. Marz, and F. Altmann. 1986. Characteristics of the asparagine-linked oligosaccharide from honey-bee venom phospholipase A2. Comp. Biochem. Physiol. 83B:321-324.

18. Weber, A., H. Schroder, K. Thalberg, and L. Marz. 1987. Specific interaction of IgE antibodies with a carbohydrate epitope of honey bee venom phospholipase A2. Allergy 35 42:464-470.

19. Stanworth, D. R., K. J. Dorrington, T. E. Hugli, K. Reid, and M. W. Turner. 1990. Nomenclature for synthetic peptides representative of immunoglobulin chain sequences. Bulletin WHO 68:109-111.

20. Rafnar, T., I. J. Griffith, M. C. Kuo, J. F. Bond, B. L. Rogers, and D.G. Klapper. 1991. Cloning of Amb a I (Antigen E), the major allergen family of short ragweed pollen. J. Biol. Chem. 266: 1229-1236.

21. Rogers, B. L., J. P. Morgenstern, I. J. Griffith, X. B. Yu, C. M. Counsell, A. W. Brauer, T. P. King, R. D. Garman, and M. C. Kuo. 1991. Complete sequence of the allergen Amb a II: recombinant expression and reactivity with T cells from ragweed allergic patients. J. Immmunol. 147:2547-2552.

22. Klapper, D. G., L. Goodfriend, and J. D. Capra. 1980. Amino acid sequence of ragweed allergen Ra3. Biochemistry 19:5729-5734.

23. Ghosh, B., M. P. Perry, T. Rafnar, and D. G. Marsh. 1993. Cloning and expression of immunologically active recombinant Amb a V allergen of short ragweed (Ambrosia artemisiifolia) pollen. J. Immunol. 150:5391-5399.

24. Roebber, M., R. Hussain, D. G. Klapper, and D. G. Marsh. 1983. Isolation and properties of a new short ragweed pollen allergen, Ra6. J. Immunol. 131 :706-711.

25. Lubahn, B., and D. G. Klapper. 1993. Cloning and characterization of ragweed allergen Arrb a VI (abst). J. Allergy Clin. Immunol. 91:338.

26. Roebber, M., and D. G. Marsh. 1991. Isolation and characterization of allergen Amb a VII from short ragweed pollen. J. Allergy Clin. Immunol. 87:324.

27. Rogers, B. L., J. Pollock, D. G. Klapper, and I. J. Griffith. 1993. Cloning, complete sequence, and recombinant expression of a novel allergen from short ragweed pollen (abst). J. Allergy Clin. Immunol. 91:339.

28. Goodfriend, L., A. M. Choudhury, D. G. Klapper, K. M. Coulter, G. Dorval, J. DelCarpio, and C. K. Osterland. 1985. RaSG, a homologue of Ra5 in giant ragweed pollen: isolation, HLA-DR-associated activity and amino acid sequence. Mol. Immunol. 22:899-906.

28A. Breitenbach M, pers. comm.

29. Nilsen, B. M., K. Sletten, M. O'Neill, B. Smestead Paulsen, and H. van Halbeek. 1991. Structural analysis of the glycoprotein allergen Art v II from pollen of mugwort (Artemesia vulgaris). J. Biol. Chem. 266:2660-2668.

29Jimenez A, Moreno C, Martinez J, Martinez A, Bartolome B, Guerra F, Palacios R 1994. Sensitization to sunflower pollen: only an occupational allergy? Int Arch Allergy Immunol 105:297-307.

30. Smith, P. M., Suphioglu,C., Griffith,I.J., Theriault,K., Knox,R. B. and Singh,M. B. 1996. Cloning and expression in yeast Pichia pastoris of a biologically active form of Cyn d 1, the major allergen of Bermuda grass pollen. J. Allergy Clin. Imnmunol. 98:331-343.

31. Suphioglu,C., Ferreira,F. and Knox,R. B. 1997. Molecular cloning and immunological characterisation of Cyn d 7, a novel calcium-binding allergen from Bermuda grass pollen. FEBS Lett. 402:167-172.

31a. Asturias J A, Arilla M C, Gomez-Bayon N, Martinez J, Martinez A, and Palacios R. 1997. Cloning and high level expression of Cynodon dactylon (Bermuda grass) pollen profilin (Cyn d 12) in Escherichia coli: purification and characterization of the allergen. Clin Exp Allergy 27:1307-1313.

32. Mecheri, S., G. Peltre, and B. David. 1985. Purification and characterization of a major allergen from Dactylis glomerata pollen: The Ag Dg 1. Int. Arch. Allergy Appl. Immunol. 78:283-289.

33. Roberts, A. M., L. J. Bevan, P. S. Flora, I. Jepson, and M. R. Walker. 1993. Nucleotide sequence of cDNA encoding the Group II allergen of Cocksfoot/Orchard grass (Dactylis glomerata), Dac g II. Allergy 48:615-623.

33a. Guerin-Marchand,C., Senechal,H., Bouin,A. P., Leduc-Brodard,V., Taudou,G., Weyer,A., Peltre,G. and David,B. 1996. Cloning, sequencing and immunological characterization of Dac g 3, a major allergen from Dactylis glomerata pollen. Mol. Immunol. 33:797-806.

34. Klysner, S., K. Welinder, H. Lowenstein, and F. Matthiesen. 1992. Group V allergens in grass pollen IV. Similarities in amino acid compositions and amino terminal sequences of the group V allergens from Lolium perenne, Poa pratensis and Dactylis glomerata. Clin. Exp. Allergy 22: 491-497.

35. Perez, M., G. Y. Ishioka, L. E. Walker, and R. W. Chesnut. 1990. cDNA cloning and immunological characterization of the rye grass allergen Lol p I. J. Biol. Chem. 265:16210-16215.

36. Griffith, I. J., P. M. Smith, J. Pollock, P. Theerakulpisut, A. Avjioglu, S. Davies, T. Hough, M. B. Singh, R. J. Simpson, L. D. Ward, and R. B. Knox. 1991. Cloning and sequencing of Lol p I, the major allergenic protein of rye-grass pollen. FEBS Letters 279:210-215.

37. Ansari, A. A., P. Shenbagamurthi, and D. G. Marsh. 1989. Complete amino acid sequence of a Lolium perenne (perennial rye grass) pollen allergen, Lol p II. J. Biol. Chem. 264:11181-11185.

37a. Sidoli,A., Tamborini,E., Giuntini,I., Levi,S., Volonte,G., Paini,C., De Lalla,C., Siccardi,A. G., Baralle,F. E., Galliani,S. and Arosio,P. 1993. Cloning, expression, and immunological characterization of recombinant Lolium perenne allergen Lol p II. J. Biol. Chem. 268:21819-21825.

38. Ansari, A. A., P. Shenbagamurthi, and D. G. Marsh. 1989. Complete primary structure of a Lolium perenne (perennial rye grass) pollen allergen, Lol p III: Comparison with known Lol p I and II sequences. Biochemistry 28:8665-8670.

39. Singh, M. B., T. Hough, P. Theerakulpisut, A. Avjioglu, S. Davies, P. M. Smith, P. Taylor, R. J. Simpson, L. D. Ward, J. McCluskey, R. Puy, and R.B. Knox. 1991. Isolation of cDNA encoding a newly identified major allergenic protein of rye-grass pollen: Intracellular targeting to the amyloplost. Proc. Natl. Acad. Sci. 88:1384-1388.

39a. van Ree R, Hoffman D R, van Dijk W, Brodard V, Mahieu K, Koeleman C A, Grande M, van Leeuwen W A, Aalberse R C. 1995. Lol p XI, a new major grass pollen allergen, is a member of a family of soybean trypsin inhibitor-related proteins. J Allergy Clin Immunol 95:970-978.

40. Suphioglu,C. and Singh,M. B. 1995. Cloning, sequencing and expression in Escherichia coli of Pha a 1 and four isoforms of Pha a 5, the major allergens of canary grass pollen. Clin. Exp. Allergy 25:853-865.

41. Dolecek,C., Vrtala,S., Laffer,S., Steinberger,P., Kraft,D., Scheiner,O. and Valenta,R. 1993. Molecular characterization of Phl p II, a major timothy grass (Phleum pratense) pollen allergen. FEBS Lett. 335:299-304.

41A. Fischer S, Grote M, Fahlbusch B, Muller WD, Kraft D, Valenta R. 1996. Characterization of Phl p 4, a major timothy grass (Phleum pratense) pollen allergen. J Allergy Clin Immunol 98:189-198.

42. Matthiesen, F., and H. Lowenstein. 1991. Group V allergens in grass pollens. I. Purification and characterization of the group V allergen from Phleum pratense pollen, Phl p V. Clin. Exp. Allergy 21:297-307.

43. Petersen,A., Bufe,A., Schramm,G., Schlaak,M. and Becker,W. M. 1995. Characterization of the allergen group VI in timothy grass pollen (Phi p 6). II. cDNA cloning of Phl p 6 and structural comparison to grass group V. Int. Arch. Allergy Immunol. 108:55-59.

44. Valenta,R., Ball,T., Vrtala,S., Duchene,M., Kraft,D. and Scheiner,O. 1994. cDNA cloning and expression of timothy grass (Phleum pratense) pollen profilin in Escherichia coli: comparison with birch pollen profilin. Biochem. Biophys. Res. Cornmun. 199:106-118.

46. Esch, R. E., and D. G. Klapper. 1989. Isolation and characterization of a major cross-reactive grass group I allergenic determinant. Mol. Immunol. 26:557-561.

47. Olsen, E., L. Zhang, R. D. Hill, F. T. Kisil, A. H. Sehon, and S. Mohapatra. 1991. Identification and characterization of the Poa p IX group of basic allergens of Kentucky bluegrass pollen. J. Immunol. 147:205-211.

48. Avjioglu, A., M. Singh, and R. B. Knox. 1993. Sequence analysis of Sor h I, the group I allergen of Johnson grass pollen and it comparison to rye-grass Lol p I (abst). J. Allergy Clin. Immunol. 91:340.

51. Larsen, J. N., P. Str'man, and H. Ipsen. 1992. PCR based cloning and sequencing of isogenes encoding the tree pollen major allergen Car b I from Carpinus betulus, hombeam. Mol. Immunol. 29:703-711.

52. Kos T, Hoffmann-Sommergruber K, Ferreira F, Hirschwehr R, Ahom H, Horak F, Jager S, Sperr W V, Kraft D, Scheiner O. 1993. Purification, characterization and N-terminal amino acid sequence of a new major allergen from European chestnut pollen—Cas s 1. Biochem Biophys Res Commun 196:1086-92.

53. Breiteneder, H., F. Ferreira, K. Hoffman-Sommergruber, C. Ebner, M. Breitenbach, H. Rumpold, D. Kraft, and O. Scheiner. 1993. Four recombinant isoforms of Cor a I, the major allergen of hazel pollen. Europ. J. Biochem. 212:355-362.

54. Ipsen, H., and B. C. Hansen. 1991. The NH2-terminal amino acid sequence of the immunochemically partial identical major allergens of alder (Alnus glutinosa) Aln g I, birch (Betula verrucosa) Bet v I, hombeam (Carpinus betulus) Car b I and oak (Quercus alba) Que a I pollens. Mol. Immunol. 28:1279-1288.

55. Taniai, M., S. Ando, M. Usui, M. Kurimoto, M. Sakaguchi, S. Inouye, and T. Matuhasi. 1988. N-terminal amino acid sequence of a major allergen of Japanese cedar pollen (Cry j I). FEBS Lett. 239:329-332.

56. Griffith, I. J., A. Lussier, R. Garman, R. Koury, H. Yeung, and J. Pollock. 1993. The cDNA cloning of Cry j I, the major allergen of Cryptomeria japonica (Japanese cedar) (abst). J. Allergy Clin. Immunol. 91:339.

57. Sakaguchi, M., S. Inouye, M. Taniai, S. Ando, M. Usui, and T. Matuhasi. 1990. Identification of the second major allergen of Japanese cedar pollen. Allergy 45:309-312.

58 Gross G N, Zimburean J M, Capra J D 1978. Isolation and partial characterization of the allergen in mountain cedar pollen. Scand J Immunol 8:437-41

58A Obispo T M, Melero J A, Carpizo J A, Carreira J, Lombardero M 1993. The main allergen of Olea europaea (Ole e I) is also present in other species of the oleaceae family. Clin Exp Allergy 23:311-316.

59. Cardaba, B., D. Hernandez, E. Martin, B. de Andres, V. del Pozo, S. Gallardo, J.C. Fernandez, R. Rodriguez, M. Villalba, P. Palomino, A. Basomba, and C. Lahoz. 1993. Antibody response to olive pollen antigens: association between HLA class II genes and IgE response to Ole e I (abst). J. Allergy Clin. Immunol. 91:338.

60. Villalba, M., E. Batanero, C. Lopez-Otin, L. M. Sanchez, R. I. Monsalve, M. A. Gonzalez de la Pena, C. Lahoz, and R. Rodriguez. 1993. Amino acid sequence of Ole e I, the major allergen from olive tree pollen (Olea europaea). Europ. J. Biochem. 216:863-869.

60A. Asturias J A, Arilla M C, Gomez-Bayon N, Martinez J, Martinez A, Palacios R 1997. Cloning and expression of the panallergen profilin and the major allergen (Ole e I) from olive tree pollen. J Allergy Clin Immunol 100:365-372.

60B. Batanero E, Villalba M, Ledesma A Puente X S, Rodriguez R. 1996. Ole e I, an olive-tree allergen, belongs to a widespread family of pollen proteins. Eur J Biochem 241: 772-778.

61. Chua, K. Y., G. A. Stewart, and W. R. Thomas. 1988. Sequence analysisof cDNA encoding for a major house dust mite allergen, Der p I. J. Exp. Med. 167:175-182.

62. Chua, K. Y., C. R. Doyle, R. J. Simpson, K. J. Turner, G. A. Stewart, and W. R. Thomas. 1990. Isolation of cDNA coding for the major mite allergen Der p II by IgE plaque immunoassay. Int. Arch. Allergy Appl. Immunol. 91:118-123.

63. Smith W A, Thomas W R. 1996. Comparative analysis of the genes encoding group 3 allergens from Dermatophagoides pteronyssinus and Dermatophagoides farinae. Int Arch Allergy Immunol 109: 133-40.

64. Lake, F. R., L. D. Ward, R. J. Simpson, P. J. Thompson, and G. A. Stewart. 1991. House dust mite-derived amylase: Allergenicity and physicochemical characterisation. J. Allergy Clin. Immunol. 87:1035-1042.

65. Tovey, E. R., M. C. Johnson, A. L. Roche, G. S. Cobon, and B. A. Baldo. 1989. Cloning and sequencing of a cDNA expressing a recombinant house dust mite protein that binds human IgE and corresponds to an important low molecular weight allergen. J. Exp. Med. 170:1457-1462.

66. Yasueda, H., T. Shida, T. Ando, S. Sugiyama, and H. Yamakawa. 1991. Allergenic and proteolytic properties of fourth allergens from Dermatophagoides mites. In: “Dust Mite Allergens and Asthma. Report of the 2nd international workshop” A. Todt, Ed., UCB Institute of Allergy, Brussels, Belgium, pp. 63-64.

67. Shen, H.-D., K.-Y. Chua, K.-L. Lin, K.-H. Hsieh, and W.R. Thomas. 1993. Molecular cloning of a house dust mite allergen with commnon antibody binding specificities with multiple components in mite extracts. Clin. Exp. Allergy 23:934-40.

67A. O'Neil G M, Donovan G R, Baldo B A. 1994. Cloning and charaterisation of a major allergen of the house dust mite Dermatophagoides pteronyssinus, homologous with glutathione S-transferase. Biochim Biophys Acta,1219:521-528.

67B. King C, Simpson R J, Moritz R L, Reed G E, Thompson P J, Stewart GA. 1996. The isolation and characterization of a novel collagenolytic serine protease allergen (Der p 9) from the dust mite Dermatophagoides pteronyssinus. J Allergy Clin Immunol 98:739-47.

68. Lind P, Hansen O C, Horn N. 1988. The binding of mouse hybridoma and human IgE antibodies to the major fecal allergen, Der p I of D. pteronyssinus. J. Immunol. 140:4256-4262.

69. Dilworth, R. J., K. Y. Chua, and W. R. Thomas. 1991. Sequence analysis of cDNA coding for a mojor house dust allergn Der f I. Clin. Exp. Allergy 21:25-32.

70. Nishiyama, C., T. Yunki, T. Takai, Y. Okumura, and H. Okudaira. 1993. Determination of three disulfide bonds in a major house dust mite allergen, Der f II. Int. Arch. Allergy Immunol. 101:159-166.

71. Trudinger, M., K. Y. Chua, and W. R. Thomas. 1991. cDNA encoding the major dust mite allergen Der fII. Clin. Exp. Allergy 21:33-38.

72. Aki T, Kodama T, Fujikawa A, Miura K, Shigeta S, Wada T, Jyo T, Murooka Y, Oka S, Ono K. 1995. Immunochemical characteristion of recombinant and native tropomyosins as a new allergen from the house dust mite Dermatophagoides farinae. J Allergy Clin Immunol 96:74-83.

73. van Hage-Hamsten, M., T. Bergman, E. Johansson, B. Persson, H. Jornvall, B. Harfast, and S. G. O. Johansson. 1993. N-terminal amino acid sequence of major allergen of the mite lepidoglyphus destructor (abst). J. Allergy Clin. Immunol. 91:353.

74. Varela J, Ventas P, Carreira J, Barbas J A, Gimenez-Gallego G, Polo F. Primary structure of Lep d I, the main Lepidoglyphus destructor allergen. Eur J Biochem 225:93-98, 1994.

75. Schmidt M, van der Ploeg I, Olsson S, van Hage Hamsten M. The complete cDNA encoding the Lepidoglyphus destructor major allergen Lep d I. FEBS Lett 370:11-14, 1995.

76. Rautiainen J, Rytkonen M, Pelkonen J, Pentikainen J, Perola O, Virtanen T, Zeiler T, Mantyjarvi R. BDA20, a major bovine dander allergen characterized at the sequence level is Bos d 2. Submitted.

77. Gjesing B, Lowenstein H. Inmunochemistry of food antigens. Ann Allergy 53:602, 1984.

78. de Groot, H., K. G. H. Goei, P. van Swieten, and R. C. Aalberse. 1991. Affinity purification of a major and a minor allergen from dog extract: Serologic activity of afflity-purified Can f I and Can f I-depleted extract. J. Allergy Clin. Immunol. 87:1056-1065.

79. Konieczny, A. Personal communication; Immunologic Pharmaceutical Corp.

79A. Bulone, V. 1998. Separation of horse dander allergen proteins by two-dimensional electrophoresis. Molecular characterisation and identification of Equ c 2.0101 and Equ c 2.0102 as lipocalin proteins. Eur J Biochem 253:202-211.

79B. Swiss-Prot acc. P81216, P81217.

80. McDonald, B., M. C. Kuo, J. L. Ohman, and L. J. Rosenwasser. 1988. A 29 amino acid peptide derived from rat alpha 2 euglobulin triggers murine allergen specific human T cells (abst). J. Allergy Clin. Immunol. 83:251.

81. Clarke, A. J., P. M. Cissold, R. A. Shawi, P. Beattie, and J. Bishop. 1984. Structure of mouse urinary protein genes: differential splicing configurations in the 3′-non-coding region. EMBO J 3:1045-1052.

82. Longbottom, J. L. 1983. Chracterization of allergens from the urines of experimental animals. McMillan Press, London, pp. 525-529.

83. Laperche, Y., K. R. Lynch, K. P. Dolans, and P. Feigelsen. 1983. Tissue-specific control of alpha 2u globulin gene expression: constitutive synthesis in submaxillary gland. Cell 32:453-460.

83A. Aukrust L, Borch S M. 1979. Partial purification and characterization of two Cladosporium herbarum allergens. Int Arch Allergy Appl Immunol 60:68-79.

83B. Sward-Nordmo M, Paulsen B S, Wold J K. 1988. The glycoprotein allergen Ag-54 (Cla h II) from Cladosporium herbarum. Structural studies of the carbohydrate moiety. Int Arch Allergy Appl Immunol 85:288-294.

84. Shen, et al. J. Allergy Clin. Immunol. 103:S157, 1999.

84A. Crameri R. Epidemiology and molecular basis of the involvement of Aspergillus fumigatus in allergic diseases. Contrib. Microbiol. Vol. 2, Karger, Basel (in press).

84B. Shen, et al. (manuscript submitted), 1999

84C. Shen H D, Ling W L, Tan M F, Wang S R, Chou H, Han SIH. Vacuolar serine proteinase: A major allergen of Aspergillus fumigatus. 10th International Congress of Immunology, Abstract, 1998.

85. Kumar, A., L. V. Reddy, A. Sochanik, and V. P. Kurup. 1993. Isolation and characterization of a recombinant heat shock protein of Aspergillus fumigatus. J. Allergy Clin. Immunol. 91:1024-1030.

86. Teshima, R., H. Ikebuchi, J. Sawada, S. Miyachi, S. Kitani, M. Iwama, M. Irie, M. Ichinoe, and T. Terao. 1993. Isolation and characterization of a major allergenic component (gp55) of Aspergillus fumigatus. J. Allergy Clin. Immunol. 92:698-706.

86A. Shen H D, Lin W L, Tsai J J, Liaw S F, Han S H. 1996. Allergenic components in three different species of Penicillium: crossreactivity among major allergens. Clin Exp Allergy 26:444-451.

86B. Shen, et al. Abstract; The XVIII Congress of the European Academy of Allergology and Clinical Immunology, Brussels, Belgium, 3-7 July 1999.

87. Shen H D, Liaw S F, Lin W L, Ro L H, Yang H L, Han S H. 1995. Molecular cloning of cDNA coding for the 68 kDa allergen of Penicillium notatum using MoAbs. Clin Exp Allergy 25:350-356.

88. Shen, H. D., K. B. Choo, H. H. Lee, J. C. Hsieh, and S. H. Han. 1991. The 40 kd allergen of Candida albicans is an alcohol dehydrogenease: molecular cloning and immunological analysis using monoclonal antibodies. Clin. Exp. Allergy 21:675-681.

89. Shen, et al. Clin. Exp. Allergy (in press), 1999.

90. Woodfolk J A, Wheatley L M, Piyasena R V, Benjamin D C, Platts-Mills TA.1998. Trichophyton antigens associated with IgE antibodies and delayed type hypersensitivity. Sequence homology to two families of serine proteinases. J Biol Chem 273:29489-96.

91. Deuell, B., L. K. Arruda, M. L. Hayden, M. D. Chapman and T. A. E. Platts-Mills. 1991. Trichophyton tonsurans Allergen I. J. ImnimLol. 147:96-101.

91A. Schmidt M, Zargari A, Holt P, Lindbom L, Hellman U, Whitley P, van der Ploeg I, Harfast B, Scheynius A. 1997. The complete cDNA sequence and expression of the first major allergenic protein of Malassezia furfur, Mal f 1. Eur J Biochem 246:181-185.

91B. Homer W E, Reese G, Lehrer S B. 1995. Identification of the allergen Psi c 2 from the basidiomycete Psilocybe cubensis as a fungal cyclophilin. Int Arch Allergy Immunol 107:298-300.

92. Kuchler, K., M. Gmachl, M. J. Sippl, and G. Kreil. 1989. Analysis of the cDNA for phospholipase A2 from honey bee venom glands: The deduced amino acid sequence reveals homology to the corresponding vertebrate enzymes. Eur. J. Biochem. 184:249-254.

93. Gmachl, M., and G. Kreil. 1993. Bee venom hyaluronidase is homologous to a membrane protein of mammalian sperm. Proc. Natl. Acad. Sci. USA 90:3569-3573.

94. Habermann, E. 1972. Bee and wasp venoms. Science 177:314-322.

95. Jacobson, R. S., and D. R. Hoffinan. 1993. Characterization of bumblebee venom allergens (abst). J. Allergy Clin. Immunol. 91:187.

96. Arruda L K, Vailes L D, Mann B J, Shannon J, Fox J W, Vedvick T S, Hayden M L, Lis Chapman M D. Molecular cloning of a major cockroach (Blattella germanica) allergen, Bla g 2. Sequence homology to the aspartic proteases. J Biol Chem 270:19563-19568, 1995.

97. Arruda L K, Vailes L D, Hayden M L, Benjamin D C, Chapman M D. Cloning of cockroach allergen, Bla g 4, identifies ligand binding proteins (or calycins) as a cause of IgE antibody responses. J Biol Chem 270:31196-31201, 1995.

98. Arruda L K, Vailes L D, Benjamin D C, Chapman M D. Molecular cloning of German Cockroach (Blattella germanica) allergens. Int Arch Allergy Immunol 107:295-297, 1995.

98A. Wu C H, Lee M F, Liao SC. 1995. Isolation and preliminary characterization of cDNA it encoding American cockroach allergens. J Allergy Clin Immtnol 96: 352-9.

99. Mazur, G., X. Baur, and V. Liebers. 1990. Hypersensitivity to hemoglobins of the Diptera family Chironomidae: Structural and functional studies of their immunogenic/allergenic sites. Monog. Allergy 28:121-137.

100. Soldatova, L., L. Kochoumian, and T. P. King. 1993. Sequence similarity of a hornet (D. maculata) venom allergen phospholipase A1 with mammalian lipases. FEBS Letters 320:145-149.

101. Lu, G., L. Kochoumian and T. P. King. Whiteface hornet venom allergen hyaluronidase: cloning and its sequence similarity with other proteins (abst.). 1994. J. Allergy Clin. Immunol. 93:224.

102. Fang, K. S. F., M. Vitale, P. Fehlner, and T. P. King. 1988. cDNA cloning and primary structure of a white-faced hornet venom allergen, antigen 5. Proc. Natl. Acad. Sci., USA 85:895-899.

103. King, T. P., D. C. Moran, D. F. Wang, L. Kochoumian, and B. T. Chait. 1990. Structural studies of a hornet venom allergen antigen 5, Dol m V and its sequence similarity with other proteins. Prot. Seq. Data Anal. 3:263-266.

104. Lu, G., M. Villalba, M. R. Coscia, D. R. Hoffman, and T. P. King. 1993. Sequence analysis and antigen cross reactivity of a venom allergen antigen 5 from hornets, wasps and yellowjackets. J. Immunol. 150: 2823-2830.

105. King, T. P. and Lu, G. 1997. Unpublished data.

105A. King T P, Lu G, Gonzalez M, Qian N and Soldatova L. 1996. Yellow jacket venom allergens, hyaluronidase and phospholipase: sequence similarity and antigenic cross-reactivity with their hornet and wasp homologs and possible implications for clinical allergy. J. Allergy Clin. Immunol. 98:588-600.

106. Hoffman, D. R. 1993. Allergens in hymenoptera venom XXV: The amino acid sequences of antigen 5 molecules and the structural basis of antigenic cross-reactivity. J. Allergy Clin. Immunol. 92:707-716.

107. Hoffman, D. R. 1992. Unpublished data. 108. Hoffman, D. R. 1993. The complete amino acid sequence of a yellowjacket venom phospholipase (abst). J. Allergy Clin. Immunol. 91:187.

109. Jacobson, R. S., D. R. Hoffman, and D. M. Kemeny. 1992. The cross-reactivity between bee and vespid hyaluronidases has a structural basis (abst). J. Allergy Clin. Immunol. 89:292.

110. Hoffman, D. R. 1993. Allergens in Hymenoptera venom XXIV: The amino acid sequences of imported fire ant venom allergens Sol i II, Sol i III, and Sol i IV. J. Allergy Clin. Immunol. 91:71-78.

111. Schmidt, M., R. B. Walker, D. R. Hoffiman, and T. J. McConnell. 1993. Nucleotide sequence of c-DNA encoding the fire ant venom protein Sol i II. FEBS Letters 319:138-140.

112. Elsayed S, Bennich H. The primary structure of Allergen M from cod. Scand J Immunol 3:683-686,1974.

113. Elsayed S, Aas K, Sletten K, Johansson SGO. Tryptic cleavage of a homogeneous cod fish allergen and isolation of two active polypeptide fragments. Immunochemistry 9:647-661, 1972.

114. Hoffman, D. R. 1983. Immunochemical identification of the allergens in egg white. J. Allergy Clin. Immunol. 71:481-486.

115. Langeland, T. 1983. A clinical and immunological study of allergy to hen's egg white. IV. specific IgE antibodies to individual allergens in hen's egg white related to clinical and immunolgical parameters in egg-allergic patients. Allergy 38:493-500.

116Daul, C. B., M. Slattery, J. E. Morgan, and S. B. Lehrer. 1993. Common crustacea allergens: identification of B cell epitopes with the shrimp specific monoclonal antibodies. In: “Molecular Biology and Immunology of Allergens” (D. Kraft and A. Sehon, eds.). CRC Press, Boca Raton. pp. 291-293.

117. K. N. Shanti, B. M. Martin, S. Nagpal, D. D. Metcalfe, P. V. Subba Rao. 1993. Identification of tropomyosin as the major shrimp allergen and characterization of its IgE-binding epitopes. J. Immunol. 151:5354-5363. 117A. M. Miyazawa, H. Fukamachi, Y. Inagaki, G. Reese, C. B. Daul, S. B. Lehrer, S. Inouye, M. Sakaguchi. 1996. Identification of the first major allergen of a squid (Todarodes , pacificus). J. Allergy Clin. Immunol. 98:948-953.

117B A. Lopata et al. 1997. Characteristics of hypersensitivity reactions and identification of a uniques 49 kDa IgE binding protein (Hal-m-1) in Abalone (Haliotis midae). J.Allergy , Clin. Immunol. Submitted

118. Monsalve, R. I., M. A. Gonzalez de la Pena, L. Menendez-Arias, C. Lopez-Otin, M. Villalba, and R. Rodriguez. 1993. Characterization of a new mustard allergen, Bra j IE. Detection of an allergenic epitope. Biochem. J. 293:625-632.

119. Mena, M., R. Sanchez-Monge, L. Gomez, G. Salcedo, and P. Carbonero. 1992. A major barley allergen associated with baker's asthma disease is a glycosylated monomeric inhibitor of insect alpha-amylase: cDNA cloning and chromosomal location of the gene. Plant Molec. Biol. 20:451-458.

120. Menendez-Arias, L., I. Moneo, J. Dominguez, and R. Rodriguez. 1988. Primary structure of the major allergen of yellow mustard (Sinapis alba L.) seed, Sin a I. Eur. J. Biochem. 177:159-166.

121. Gonzalez R, Varela J, Carreira J, Polo F. Soybean hydrophobic protein and soybean hull allergy. Lancet 346:48-49, 1995.

122. Christie, J. F., B. Dunbar, I. Davidson, and M. W. Kennedy. 1990. N-terminal amino acid sequence identity between a major allergen of Ascaris lumbricoides and Ascaris suum and MHC-restricted IgE responses to it. Immunology 69:596-602.

123. Czuppon A B, Chen Z, Rennert S, Engelke T, Meyer HE, Heber M, Baur X. The fit rubber elongation factor of rubber trees (Hevea brasiliensis) is the major allergen in latex. J Allergy Clin Immunol 92:690-697, 1993.

124. Attanayaka DPSTG, Kekwick RGO, Franklin FCH. 1991. Molecular cloning and nucleotide sequencing of the rubber elongation factor gene from hevea brasiliensis. Plant Mol Biol 16:1079-1081.

125. Chye M L, Cheung K Y. 1995. (1,3-glucanase is highly expressed in Laticifers of Hevea brasiliensis. Plant Mol Biol 26:397-402.

126. Alenius H, Palosuo T, Kelly K, Kurup V, Reunala T, Makinen-Kiljunen S, Tuijanmaa K Fink J. 1993. IgE reactivity to 14-kD and 27-kD natural rubber proteins in Latex-allergic children with Spina bifida and other congenital anomalies. Int Arch Allergy Immunol 102:61-66.

127. Yeang H Y, Cheong K F, Sunderasan E, Hamzah S, Chew N P, Hamid S, Hamilton R G, Caidosa M J. 1996. The 14.6 kD (REF, Hev b 1) and 24 kD (Hev b 3) rubber particle proteins are recognized by IgE from Spina Bifida patients with Latex allergy. J Allerg Clin Immunol in press.

128. Sunderasan E, Hamzah S, Hamid S, Ward M A, Yeang H Y, Cardosa M J. 1995. Latex B-serum (-1,3-glucanase (Hev b 2) and a component of the microhelix (Hev b 4) are major Latex allergens. J nat Rubb Res 10:82-99.

What is claimed is:

1. A non-human animal that (1) has an irritated GI tract; and (2) has been manipulated by the hand of man to be allergic to an orally-delivered antigen.

2. A non-human animal that has an allergic response to an orally-delivered antigen, wherein the allergic response is mediated by antigen-specific IgE antibodies.

3. A non-human animal that has an allergic response to an orally-delivered antigen and has antigen-specific IgE levels of at least about 500 ng/ml.

4. The non-human animal of claim 3 wherein the antigen-specific IgE levels are at least 1000ng/ml.

5. The non-human animal of claim 3 wherein the antigen-specific IgE levels are at least 1500 ng/ml.

6. A non-human animal characterized in that, when presented with a potential sensitizing antigen in combination with cholera toxin, the animal either develops an immune reaction characterized by anaphylaxis to oral challenged with the antigen or does not develop such a response, the development or non-development of a response being predictors of the likelihood that human subjects will anaphylax to oral exposure to the antigen.

7. A kit comprising:
the non-human animal of claim 2 or claim 6; and
reagents to detect antigen-specific IgE.

8. A kit comprising:
a non-human animal characterized or that
1) the animal has an irritated GI tract; and
2) when exposed to peanut in carbirati with choleatoxin adminstered, the animal becomes sensitized and will araphylax to orally-adminstered peanut; and sensitizing reagents including choleatoxin.

9. The kit of claim 6 wherein the aminal demonstrates an ability to be sensitized to orally-delivered anigens to which humans become sensitized, but not to orally-delivered antigens to which humans do not become sensitized.

10. The kit of claim 9 wherein the orally-delivered antigens to which humans do not become sensitized include those to which less than 10% of the human population.

11. The kit of claim 9 wherein the orally-delivered antigens to which humans do not become sensitized include those to which less than 1% of the human population.

11a. The kit of claim 9 wherein the orally-delivered antigens to which humans do not become sensitized include corn.

12. The animal of claim 1 wherein the animal anaphylaxes when contacted with the sensitizing antigen.

13. The animal of claim 1 wherein the animal is a non-human mammal.

14. The animal of claim 1 wherein the animal is a rodent.

15. The animal of claim 1 wherein the animal is selected from the group consisting of rat, mouse, rabbit, ferret, hamster, and guinea pig.

16. The animal of claim 1 wherein the animal is a rat.

17. The animal of claim 1 wherein the animal is a mouse.

18. The animal of claim 1 wherein the animal is a C3H/HeJ mouse.

19. The animal of claim 1 wherein the animal is a non-hunan primate.

20. The animal of claim 1 wherein the animal is selected from the group consisting of baboons, monkeys, gorillas, apes, and orangutans.

21. The animal of claim 1 wherein the antigen to which the animal is allergic is one to which at least one human is allergic and that poses an anaphylactic risk to at least one allergic human.

22. The animal of claim 1 wherein the antigen is an anaphylactic antigen.

23. The animal of claim 1 wherein the antigen is a food antigen.

24. The animal of claim 23 wherein the food antigen is selected from the groups consisting of fruit antigens, berry antigens, nut antigens, bean antigens, milk antigens, dairy product antigens, peanut antigens, seed antigens, fish antigens, and shellfish antigens.

25. The animal of claim 23 wherein the food antigen is a peanut antigen.

26. The animal of claim 23 wherein the food antigen is a milk antigen.

27. The animal of claim 1 wherein the antigen is an environmental antigen.

28. The animal of claim 27 wherein the environmental antigen is selected from the groups consisting of weed pollen antigens, grass pollen antigens, tree pollen antigens, mite antigens, animal antigens, animal dander antigens, fungal antigens, and insect antigens.

29. The animal of claim 27 wherein the environmental antigen is a latex antigen.

30. The animal of claim 1 wherein the antigen is a pharmaceutical agent.

31. The animal of claim 1 wherein the the animal does not have an allergic response to non-allergenic food antigens.

32. The animal of claim 31 wherein the non-allergenic antigens are derived from corn.

33. A method of using a sensitized an animal to detect the presence of antigen, the method comprising the steps of:
providing an animal that is sensitized to an antigen and that is not able to be sensitized to antigens that humans are not normally allergic;
providing a test product;
contacting animal with test product; and
determining immune response of animal.

34. The method of claim 33 wherein the antigen that humans are not normally allergic comprises antigens derived from corn.

35. The method of claim 33 wherein the test product is a food product.

36. The method of claim 33 wherein the test product is a skin product.

37. The method of claim 33 wherein the test product is a pharmaceutical composition.

37a. The method of claim 33 wherein the step of contacting comprises adminstering the test product orally.

38. A method of assessing antigenicity of a test substance, the method comprising steps of:
providing a non-human animal as a model system;
exposing the non-human animal to a test substance to which one or more humans are expected to become exposed via a particular route, the exposure to the non-human animal being via the same route; and
determining whether the non-human animal can be made to be allergic to the test substance.

39. The method of claim 38, wherein the step of providing comprises providing a non-human animal that is susceptible to sensitization to antigens known to be allergenic in humans so that sensitized animals display an immune response to the antigens that has hallmarks characteristic of allergic responses observed in humans.

40. The method of claim 39, wherein the hallmarks are selected from the group consisting of elevated antigen-specific IgE levels above about 500 ng/ml, elevated antigen-specific IgE levels above about 1000 ng/ml, elevated antigen-specific IgE levels above about 1500 ng/ml, anaphylaxis, hives, drop in body temperature, diarrhea, difficulty breathing, increased mast cell degranulation, increased histamine release, increase airway resistance, and death.

41. A method of sensitizing an animal to an antigen, the method comprising steps of:
providing an animal, wherein the animal has an inflamed gastrointestinal tract; and
administering a sensitizing composition to the animal.

42. The method of claim 41, wherein the animal is infected with a virus.

43. The method of claim 42, wherein the virus is murine mammary tumor virus (MMTV).

44. The method of claim 41, wherein the animal is infected with bacteria.

45. The method of claim 41, wherein the animal is exposed to a chemical compound that causes gastrointestinal inflammation.

46. The method of claim 45, wherein the chemical compound is an organic compound.

47. The method of claim 45, wherein the chemical compound is ethanol.
